SEQUENCE LISTING (113 sequences)

SEQ ID NO: 1 - protein, 14 aa, synthetic construct
  REGION 1..14: note = Sequence motif 1
  VARIANT 5: note = misc_feature - Xaa can be any naturally occurring amino acid
  VARIANT 7..8: note = misc_feature - Xaa can be any naturally occurring amino acid
  VARIANT 10: note = MISC_FEATURE - Xaa can be tyrosine or methionine
  VARIANT 11..12: note = misc_feature - Xaa can be any naturally occurring amino acid
  VARIANT 13: note = MISC_FEATURE - Xaa can be methionine, valine or cysteine
LRPMXAXXLXXXXR

SEQ ID NO: 2 - protein, 14 aa, synthetic construct
  REGION 1..14: note = Sequence motif 2
  VARIANT 3: note = misc_feature - Xaa can be any naturally occurring amino acid
  VARIANT 8: note = misc_feature - Xaa can be any naturally occurring amino acid
  VARIANT 10: note = misc_feature - Xaa can be any naturally occurring amino acid
  VARIANT 11: note = MISC_FEATURE - Xaa can geglycine or alanine
  VARIANT 12..13: note = misc_feature - Xaa can be any naturally occurring amino acid
HLXEFTMXNXXXXG

SEQ ID NO: 3 - protein, 7 aa, synthetic construct
  REGION 1..7: note = Sequence motif3
  VARIANT 3..4: note = misc_feature - Xaa can be any naturally occurring amino acid
  VARIANT 6: note = misc_feature - Xaa can be any naturally occurring amino acid
VYXXTXD

SEQ ID NO: 4 - protein, 10 aa, synthetic construct
  REGION 1..10: note = Sequence motif 4
  VARIANT 2..4: note = misc_feature - Xaa can be any naturally occurring amino acid
  VARIANT 7: note = MISC_FEATURE - Xaa can be arginine or isoleucine
  VARIANT 7: note = MISC_FEATURE - Xaa can be arginine, isoleucine or asparagine
  VARIANT 8..9: note = misc_feature - Xaa can be any naturally occurring amino acid
SXXXGPXXXD

SEQ ID NO: 5 - skipped

SEQ ID NO: 6 - protein, 10 aa, synthetic construct
  REGION 1..10: note = Sequence motif 6
  VARIANT 2: note = MISC_FEATURE - Xaa can be alanine, leucine or isoleucine
GXGFGLERLL

SEQ ID NO: 7 - protein, 14 aa, synthetic construct
  REGION 1..14: note = Sequence MotifM1a
LRPMLAPNLYNYMR

SEQ ID NO: 8 - protein, 14 aa, synthetic construct
  REGION 1..14: note = Sequence Motif M1b
LRPMLAPTLYNYMR

SEQ ID NO: 9 - protein, 14 aa, synthetic construct
  REGION 1..14: note = Sequence Motif M1c
LRPMLAPNLYSVMR

SEQ ID NO: 10 - protein, 14 aa, synthetic construct
  REGION 1..14: note = Sequence Motif M1d
LRPMLAPNLYTLMR

SEQ ID NO: 11 - protein, 14 aa, synthetic construct
  REGION 1..14: note = Sequence motif M1e
LRPMLAPVLYNYMR

SEQ ID NO: 12 - protein, 14 aa, synthetic construct
  REGION 1..14: note = Sequence Motif M1f
LRPMHAMNLYYVMR

SEQ ID NO: 13 - protein, 14 aa, synthetic construct
  REGION 1..14: note = Sequence Motif M2a
HLEEFTMLNFGQMG

SEQ ID NO: 14 - protein, 14 aa, synthetic construct
  REGION 1..14: note = Sequence Motif M2b
HLEEFTMVNFGQMG

SEQ ID NO: 15 - protein, 14 aa, synthetic construct
  REGION 1..14: note = Sequnece Motif M2c
HLEEFTMLNLGDMG

SEQ ID NO: 16 - protein, 14 aa, synthetic construct
  REGION 1..14: note = Sequence motif M2d
HLNEFTMLNLGELG

SEQ ID NO: 17 - protein, 14 aa, synthetic construct
  REGION 1..14: note = Sequence Motif M2e
HLEEFTMVNFGQMG

SEQ ID NO: 18 - protein, 14 aa, synthetic construct
  REGION 1..14: note = Sequence Motif M2f
HLEEFTMLNLGELG

SEQ ID NO: 19 - protein, 7 aa, synthetic construct
  REGION 1..7: note = Sequence Motif M3a, b
VYGDTLD

SEQ ID NO: 20 - protein, 7 aa, synthetic construct
  REGION 1..7: note = Sequence Motif M3c
VYKETID

SEQ ID NO: 21 - protein, 7 aa, synthetic construct
  REGION 1..7: note = Sequence Motif M3d
VYGDTVD

SEQ ID NO: 22 - protein, 7 aa, synthetic construct
  REGION 1..7: note = Sequence Motif M3e
VYGNTVD

SEQ ID NO: 23 - protein, 7 aa, synthetic construct
  REGION 1..7: note = Sequence Motif M3f
VYVETLD

SEQ ID NO: 24 - protein, 10 aa, synthetic construct
  REGION 1..10: note = Sequence Motif M4a
SAVVGPRPLD

SEQ ID NO: 25 - protein, 10 aa, synthetic construct
  REGION 1..10: note = Sequence Motif M4b
SAVVGPRSLD

SEQ ID NO: 26 - protein, 10 aa, synthetic construct
  REGION 1..10: note = Sequence Motif M4c
SAAVGPRYLD

SEQ ID NO: 27 - protein, 10 aa, synthetic construct
  REGION 1..10: note = Sequence Motif M4d
SGAMGPRFLD

SEQ ID NO: 28 - protein, 10 aa, synthetic construct
  REGION 1..10: note = Sequence Motif M4e
SAVVGPRPMD

SEQ ID NO: 29 - protein, 10 aa, synthetic construct
  REGION 1..10: note = Sequence Motif M4f
SGAVGPRVLD

SEQ ID NO: 30 - protein, 7 aa, synthetic construct
  REGION 1..7: note = Sequence Motif M5a, b
WGIDKPW

SEQ ID NO: 31 - protein, 7 aa, synthetic construct
  REGION 1..7: note = Sequence Motif M5c
HDIHEPW

SEQ ID NO: 32 - protein, 7 aa, synthetic construct
  REGION 1..7: note = Sequence Motif M5d
WEIFDPW

SEQ ID NO: 33 - protein, 7 aa, synthetic construct
  REGION 1..7: note = Sequence Motif M5e
WGINKPW

SEQ ID NO: 34 - protein, 7 aa, synthetic construct
  REGION 1..7: note = Sequence Motif M5f
HDIHEPW

SEQ ID NO: 35 - protein, 10 aa, synthetic construct
  REGION 1..10: note = Sequence Motif 6a, b, c, d, e, f
GAGFGLERLL

SEQ ID NO: 36 - protein, 14 aa, synthetic construct
  REGION 1..14: note = Sequence Motif M1a*
LRPMLAPNLMNYMR

SEQ ID NO: 37 - protein, 14 aa, synthetic construct
  REGION 1..14: note = Sequence Motif M1b*
LRPMLAPTLMNYMR

SEQ ID NO: 38 - protein, 14 aa, synthetic construct
  REGION 1..14: note = Sequence Motif M1c*
LRPMLAPNLMSVMR

SEQ ID NO: 39 - protein, 14 aa, synthetic construct
  REGION 1..14: note = Sequence Motif M1d*
LRPMLAPNLMTLMR

SEQ ID NO: 40 - protein, 14 aa, synthetic construct
  REGION 1..14: note = Sequence Motif M1e*
LRPMLAPVLMNYMR

SEQ ID NO: 41 - protein, 14 aa, synthetic construct
  REGION 1..14: note = Sequence Motif M1f*
LRPMHAMNLMYVMR

SEQ ID NO: 42 - protein, 14 aa, synthetic construct
  REGION 1..14: note = Sequence Motif M2a*
HLEEFTMLNFAQMG

SEQ ID NO: 43 - protein, 14 aa, synthetic construct
  REGION 1..14: note = Sequence Motif M2b*
HLEEFTMVNFAQMG

SEQ ID NO: 44 - protein, 14 aa, synthetic construct
  REGION 1..14: note = Sequence Motif M2c*
HLEEFTMLNLADMG

SEQ ID NO: 45 - protein, 14 aa, synthetic construct
  REGION 1..14: note = Sequence Motif M2d*
HLNEFTMLNLAELG

SEQ ID NO: 46 - protein, 14 aa, synthetic construct
  REGION 1..14: note = Sequence Motif M2e*
HLEEFTMVNFAQMG

SEQ ID NO: 47 - protein, 14 aa, synthetic construct
  REGION 1..14: note = Sequence Motif M2f*
HLEEFTMLNLAELG

SEQ ID NO: 48 - protein, 10 aa, synthetic construct
  REGION 1..10: note = Sequence Motif M4a*
SAVVGPIPLD

SEQ ID NO: 49 - protein, 10 aa, synthetic construct
  REGION 1..10: note = Sequence Motif M4b*
SAVVGPISLD

SEQ ID NO: 50 - protein, 10 aa, synthetic construct
  REGION 1..10: note = Sequence Motif M4d*
SAAVGPIYLD

SEQ ID NO: 51 - protein, 10 aa, synthetic construct
  REGION 1..10: note = Sequence Motif M4d*
SGAMGPIFLD

SEQ ID NO: 52 - protein, 10 aa, synthetic construct
  REGION 1..10: note = Sequence Motif M4e*
SAVVGPIPMD

SEQ ID NO: 53 - protein, 10 aa, synthetic construct
  REGION 1..10: note = Sequence Motif M4f*
SGAVGPIVLD

SEQ ID NO: 54 - protein, 17 aa, synthetic construct
  REGION 1..17: note = Nuclear Export Signal
ACPVPLQLPPLERLTLD

SEQ ID NO: 55 - DNA, 1365 bp, Methanosarcina mazei
  source 1..1365: mol_type = genomic DNA
atggataaaaaaccgctgaataccctgatctctgctactggtctgtggatgagtcgtaccggaaccattcataaaatcaaacaccacgaggttagccgttcgaaaatctatattgagatggcgtgtggcgatcatctggttgtgaacaatagccgctcttctcgtacagcacgtgcactgcgtcaccacaaatatcgtaaaacctgtaaacgttgccgtgtgtccgatgaggatctgaacaaattcctgacaaaagccaatgaggaccaaacaagcgtgaaagtgaaagtcgttagcgctcctacccgtactaaaaaagcaatgccgaaatccgttgctcgtgcccctaaaccactggaaaacactgaagcagcacaggcacagccgtctggaagcaaattctctccggccattcctgtttctacccaggagtccgtttctgttccagcaagtgtgagcaccagcattagcagtattagcaccggtgccaccgctagcgccctggttaaaggcaataccaatccgattacaagcatgtctgccccggttcaagcatcagctccagcactgacaaaatcccaaaccgatcgtctggaggttctgctgaatccgaaagacgaaatcagcctgaattccggcaaaccgtttcgtgaactggagagcgaactgctgtcacgtcgtaaaaaagacctgcaacaaatctatgccgaagaacgtgagaactatctggggaaactggaacgtgaaatcacccgctttttcgtggatcgtggctttctggagatcaaatccccgattctgattcctctggagtatatcgagcgtatgggcatcgacaatgataccgaactgagcaaacaaattttccgtgtggataaaaacttctgtctgcgccctatgctggcaccaaatctgtataactatctgcgcaaactggaccgtgccctgcctgatcctatcaaaatcttcgagatcggcccgtgttatcgtaaagagtccgacggtaaagaacatctggaggagtttaccatgctgaacttttgccaaatgggttcaggttgtactcgtgagaacctggaaagcatcatcaccgattttctgaaccacctgggcattgacttcaaaattgtgggcgacagctgtatggtgtatggcgacaccctggatgtcatgcacggcgacctggaactgtctagtgccgttgtgggcccaatcccgctggatcgtgagtggggtatcgacaaaccttggatcggtgcgggttttggtctggagcgtctgctgaaagtaaaacacgacttcaagaacatcaaacgtgctgcacgttccgagtcctattacaatggtatttctactaacctgtaa

SEQ ID NO: 56 - protein, 454 aa, Methanosarcina mazei
MDKKPLNTLISATGLWMSRTGTIHKIKHHEVSRSKIYIEMACGDHLVVNNSRSSRTARALRHHKYRKTCKRCRVSDEDLNKFLTKANEDQTSVKVKVVSAPTRTKKAMPKSVARAPKPLENTEAAQAQPSGSKFSPAIPVSTQESVSVPASVSTSISSISTGATASALVKGNTNPITSMSAPVQASAPALTKSQTDRLEVLLNPKDEISLNSGKPFRELESELLSRRKKDLQQIYAEERENYLGKLEREITRFFVDRGFLEIKSPILIPLEYIERMGIDNDTELSKQIFRVDKNFCLRPMLAPNLYNYLRKLDRALPDPIKIFEIGPCYRKESDGKEHLEEFTMLNFCQMGSGCTRENLESIITDFLNHLGIDFKIVGDSCMVYGDTLDVMHGDLELSSAVVGPIPLDREWGIDKPWIGAGFGLERLLKVKHDFKNIKRAARSESYYNGISTNL

SEQ ID NO: 57 - DNA, 1257 bp, Methanosarcina barkeri
  source 1..1257: mol_type = genomic DNA
atggataaaaaaccgctggatgtgctgattagcgcgaccggcctgtggatgagccgcaccggcaccctgcataaaattaaacatcatgaagtgagccgcagcaaaatttatattgaaatggcgtgcggcgatcatctggtggtgaacaacagccgcagctgccgcaccgcgcgcgcgtttcgccatcataaatatcgcaaaacctgcaaacgctgccgcgtgagcgatgaagatattaacaactttctgacccgcagcaccgaaagcaaaaacagcgtgaaagtgcgcgtggtgagcgcgccgaaagtgaaaaaagcgatgccgaaaagcgtgagccgcgcgccgaaaccgctggaaaacagcgtgagcgcgaaagcgagcaccaacaccagccgcagcgtgccgagcccggcgaaaagcaccccgaacagcagcgtgccggcgagcgcgccggcgccgagcctgacccgcagccagctggatcgcgtggaagcgctgctgagcccggaagataaaattagcctgaacatggcgaaaccgtttcgcgaactggaaccggaactggtgacccgccgcaaaaacgattttcagcgcctgtataccaacgatcgcgaagattatctgggcaaactggaacgcgatattaccaaattttttgtggatcgcggctttctggaaattaaaagcccgattctgattccggcggaatatgtggaacgcatgggcattaacaacgataccgaactgagcaaacagatttttcgcgtggataaaaacctgtgcctgcgcccgatgctggcgccgaccctgtataactatctgcgcaaactggatcgcattctgccgggcccgattaaaatttttgaagtgggcccgtgctatcgcaaagaaagcgatggcaaagaacatctggaagaatttaccatggtgaacttttgccagatgggcagcggctgcacccgcgaaaacctggaagcgctgattaaagaatttctggattatctggaaattgattttgaaattgtgggcgatagctgcatggtgtatggcgataccctggatattatgcatggcgatctggaactgagcagcgcggtggtgggcccggtgagcctggatcgcgaatggggcattgataaaccgtggattggcgcgggctttggcctggaacgcctgctgaaagtgatgcatggctttaaaaacattaaacgcgcgagccgcagcgaaagctattataacggcattagcaccaacctg

SEQ ID NO: 58 - protein, 419 aa, Methanosarcina barkeri
MDKKPLDVLISATGLWMSRTGTLHKIKHHEVSRSKIYIEMACGDHLVVNNSRSCRTARAFRHHKYRKTCKRCRVSDEDINNFLTRSTESKNSVKVRVVSAPKVKKAMPKSVSRAPKPLENSVSAKASTNTSRSVPSPAKSTPNSSVPASAPAPSLTRSQLDRVEALLSPEDKISLNMAKPFRELEPELVTRRKNDFQRLYTNDREDYLGKLERDITKFFVDRGFLEIKSPILIPAEYVERMGINNDTELSKQIFRVDKNLCLRPMLAPTLYNYLRKLDRILPGPIKIFEVGPCYRKESDGKEHLEEFTMVNFCQMGSGCTRENLEALIKEFLDYLEIDFEIVGDSCMVYGDTLDIMHGDLELSSAVVGPVSLDREWGIDKPWIGAGFGLERLLKVMHGFKNIKRASRSESYYNGISTNL

SEQ ID NO: 59 - DNA, 825 bp, unidentified
  source 1..825: mol_type = unassigned DNA; note = Methanomethylophilus alvus
atgaccgtgaaatataccgatgcgcagattcagcgcctgcgcgaatatggcaacggcacctatgaacagaaagtgtttgaagatctggcgagccgcgatgcggcgtttagcaaagaaatgagcgtggcgagcaccgataacgaaaaaaaaattaaaggcatgattgcgaacccgagccgccatggcctgacccagctgatgaacgatattgcggatgcgctggtggcggaaggctttattgaagtgcgcaccccgatttttattagcaaagatgcgctggcgcgcatgaccattaccgaagataaaccgctgtttaaacaggtgttttggattgatgaaaaacgcgcgctgcgcccgatgctggcgccgaacctgtatagcgtgatgcgcgatctgcgcgatcataccgatggcccggtgaaaatttttgaaatgggcagctgctttcgcaaagaaagccatagcggcatgcatctggaagaatttaccatgctgaacctggtggatatgggcccgcgcggcgatgcgaccgaagtgctgaaaaactatattagcgtggtgatgaaagcggcgggcctgccggattatgatctggtgcaggaagaaagcgatgtgtataaagaaaccattgatgtggaaattaacggccaggaagtgtgcagcgcggcggtgggcccgcattatctggatgcggcgcatgatgtgcatgaaccgtggagcggcgcgggctttggcctggaacgcctgctgaccattcgcgaaaaatatagcaccgtgaaaaaaggcggcgcgagcattagctatctgaacggcgcgaaaattaac

SEQ ID NO: 60 - protein, 275 aa, unidentified
  source 1..275: note = Methanomethylophilus alvus
MTVKYTDAQIQRLREYGNGTYEQKVFEDLASRDAAFSKEMSVASTDNEKKIKGMIANPSRHGLTQLMNDIADALVAEGFIEVRTPIFISKDALARMTITEDKPLFKQVFWIDEKRALRPMLAPNLYSVMRDLRDHTDGPVKIFEMGSCFRKESHSGMHLEEFTMLNLVDMGPRGDATEVLKNYISVVMKAAGLPDYDLVQEESDVYKETIDVEINGQEVCSAAVGPHYLDAAHDVHEPWSGAGFGLERLLTIREKYSTVKKGGASISYLNGAKIN

SEQ ID NO: 61 - DNA, 864 bp, Desulfitobacterium hafniense
  source 1..864: mol_type = genomic DNA
atgtttctgacccgccgcgatccgccgctgagcagcttttggaccaaagtgcagtatcagcgcctgaaagaactgaacgcgagcggcgaacagctggaaatgggctttagcgatgcgctgagccgcgatcgcgcgtttcagggcattgaacatcagctgatgagccagggcaaacgccatctggaacagctgcgcaccgtgaaacatcgcccggcgctgctggaactggaagaaaaactggcgaaagcgctgcatcagcagggctttgtgcaggtggtgaccccgaccattattaccaaaagcgcgctggcgaaaatgaccattggcgaagatcatccgctgtttagccaggtgttttggctggatggcaaaaaatgcctgcgcccgatgctggcgccgaacctgtataccctgtggcgcgaactggaacgcctgtgggataaaccgattcgcatttttgaaattggcacctgctatcgcaaagaaagccagggcgcgcagcatctgaacgaatttaccatgctgaacctgaccgaactgggcaccccgctggaagaacgccatcagcgcctggaagatatggcgcgctgggtgctggaagcggcgggcattcgcgaatttgaactggtgaccgaaagcagcgtggtgtatggcgataccgtggatgtgatgaaaggcgatctggaactggcgagcggcgcgatgggcccgcattttctggatgaaaaatgggaaatttttgatccgtgggtgggcctgggctttggcctggaacgcctgctgatgattcgcgaaggcacccagcatgtgcagagcatggcgcgcagcctgagctatctggatggcgtgcgcctgaacattaac

SEQ ID NO: 62 - protein, 288 aa, Desulfitobacterium hafniense
MFLTRRDPPLSSFWTKVQYQRLKELNASGEQLEMGFSDALSRDRAFQGIEHQLMSQGKRHLEQLRTVKHRPALLELEEKLAKALHQQGFVQVVTPTIITKSALAKMTIGEDHPLFSQVFWLDGKKCLRPMLAPNLYTLWRELERLWDKPIRIFEIGTCYRKESQGAQHLNEFTMLNLTELGTPLEERHQRLEDMARWVLEAAGIREFELVTESSVVYGDTVDVMKGDLELASGAMGPHFLDEKWEIFDPWVGLGFGLERLLMIREGTQHVQSMARSLSYLDGVRLNIN

SEQ ID NO: 63 - DNA, 1215 bp, unidentified
  source 1..1215: mol_type = unassigned DNA; note = Methanosarcinaceae archaeon
atggaacgcccgaacattgaaaaactggtgaaacagaacggcgtgtggatgagccgcaacgatgtgctgcatggcgtgaaagatattaccgtgagccgcaaaaaagtgcgcattgaaatggattgcggcgattttgcggtggtgaacaacagccgccgcagcgcgagcgcgcgcgcgctgcgcaacagcaaatataaaaaagtgtgcaaacgctgcaaactgagcgatgaacgcattgaacgctttgtgaccaaaaactttagcaccccggtgcagcagaccgtgagcgtgagcaacaccggcaccaaaaaagtgaccaaaaaaaacaccgcgagcattaccccggtgccggaaaaaattgaaaaccaggtgaaaacccagcaggtgaaaacccagagcgaatataccgatagcatgaaaaaacgcattgaaatgattagcgtgccgggcgataacattgattttaccgattttgataaactggtggatctgccgccgtttgaagtgctggaaaaagaactggtggaacgccgcaaaaaagatgtgtgcaaaatttatgaaaccagccgcgaagatatgctggcggcgctggaacgcgatattagcgaatttctgcgcagcgaaggctttatggaaattaaaagcagcattctgattccggaagaatatctgctgcagatgggcattgatccggaaaccgatgaagattataaagtgtttaaaaccaacggcaacatgtatctgcgcccgatgctggcgccggtgctgtataactatctgcgccgctttgataaaattctgccggatccggtgaaaatttttgaagtgggcacctgctatcgccgcgaaagcgatagcagcaaccatctggaagaatttaccatggtgaacttttgccagatgggcgatggcgcgaacgcggaaaacctgatggaaattattgatagctttctgaaacatctgggcattgaatataccaccgaaggcgatagctgcctggtgtatggcaacaccgtggatattatgcatggcgatatggaactgagcagcgcggtggtgggcccggtgccgatggatattgattggggcattaacaaaccgtggattggcgcgggctttggcctggaacgcctgctgaaagcgaaacatggctttaaaaacgtgaaacgcggcagccgcagcgaactgtattataacggcaactatattaacttt

SEQ ID NO: 64 - protein, 405 aa, unidentified
  source 1..405: note = Methanosarcinaceae archaeon
MERPNIEKLVKQNGVWMSRNDVLHGVKDITVSRKKVRIEMDCGDFAVVNNSRRSASARALRNSKYKKVCKRCKLSDERIERFVTKNFSTPVQQTVSVSNTGTKKVTKKNTASITPVPEKIENQVKTQQVKTQSEYTDSMKKRIEMISVPGDNIDFTDFDKLVDLPPFEVLEKELVERRKKDVCKIYETSREDMLAALERDISEFLRSEGFMEIKSSILIPEEYLLQMGIDPETDEDYKVFKTNGNMYLRPMLAPVLYNYLRRFDKILPDPVKIFEVGTCYRRESDSSNHLEEFTMVNFCQMGDGANAENLMEIIDSFLKHLGIEYTTEGDSCLVYGNTVDIMHGDMELSSAVVGPVPMDIDWGINKPWIGAGFGLERLLKAKHGFKNVKRGSRSELYYNGNYINF

SEQ ID NO: 65 - DNA, 705 bp, unidentified
  source 1..705: mol_type = unassigned DNA; note = Candidatus Methanoplasma termitum
atgagcgatctggtgagcagcaacgaaaaagaaattgcgggcatgctgcgcagcccgagccgccatcagctggcggcgctggaagaagatctggcggcggcgctgattgcgcgcggctttattgaagtgaaaaccccggcgtttgtgagcgtggcgagcctggaaaaaatgaccattaccccggaacatccgctgtataaacaggtgtttatgattgatgataaacgctgcctgcgcccgatgcatgcgatgaacctgtattatgtgatgcgcaaactgcgcgatcataccgatggcccggtgaaaatttttgaaattggcagctgctttcgcaaagaaagccatagcggcagccatctggaagaatttaccatgctgaacctggtggaactgggcccggaaggcgatgcgaccgaagcgctgaaagatcatattggcgcggtgatgaaagtgaccggcctggaatataccctggtgcgcgaagaaagcgatgtgtatgtggaaaccctggatgtggaaattgatggcggcgaagtggcgagcggcgcggtgggcccgcatgtgctggataacgcgcatgatattcatgaaccgtggagcggcattggctttggcctggaacgcctgctgatgattatgaacgaaaaaagcaccgtgaaaaaaaccggccgcagcctgagctatctgaacggcgcgaaaattaac

SEQ ID NO: 66 - protein, 235 aa, unidentified
  source 1..235: note = Candidatus Methanoplasma termitum
MSDLVSSNEKEIAGMLRSPSRHQLAALEEDLAAALIARGFIEVKTPAFVSVASLEKMTITPEHPLYKQVFMIDDKRCLRPMHAMNLYYVMRKLRDHTDGPVKIFEIGSCFRKESHSGSHLEEFTMLNLVELGPEGDATEALKDHIGAVMKVTGLEYTLVREESDVYVETLDVEIDGGEVASGAVGPHVLDNAHDIHEPWSGIGFGLERLLMIMNEKSTVKKTGRSLSYLNGAKIN

SEQ ID NO: 67 - DNA, 1365 bp, synthetic construct
  misc_feature 1..1365: note = PylRS AF mutant
  source 1..1365: mol_type = other DNA
atggataaaaaaccgctgaataccctgatctctgctactggtctgtggatgagtcgtaccggaaccattcataaaatcaaacaccacgaggttagccgttcgaaaatctatattgagatggcgtgtggcgatcatctggttgtgaacaatagccgctcttctcgtacagcacgtgcactgcgtcaccacaaatatcgtaaaacctgtaaacgttgccgtgtgtccgatgaggatctgaacaaattcctgacaaaagccaatgaggaccaaacaagcgtgaaagtgaaagtcgttagcgctcctacccgtactaaaaaagcaatgccgaaatccgttgctcgtgcccctaaaccactggaaaacactgaagcagcacaggcacagccgtctggaagcaaattctctccggccattcctgtttctacccaggagtccgtttctgttccagcaagtgtgagcaccagcattagcagtattagcaccggtgccaccgctagcgccctggttaaaggcaataccaatccgattacaagcatgtctgccccggttcaagcatcagctccagcactgacaaaatcccaaaccgatcgtctggaggttctgctgaatccgaaagacgaaatcagcctgaattccggcaaaccgtttcgtgaactggagagcgaactgctgtcacgtcgtaaaaaagacctgcaacaaatctatgccgaagaacgtgagaactatctggggaaactggaacgtgaaatcacccgctttttcgtggatcgtggctttctggagatcaaatccccgattctgattcctctggagtatatcgagcgtatgggcatcgacaatgataccgaactgagcaaacaaattttccgtgtggataaaaacttctgtctgcgccctatgctagcaccaaatctggctaactatctgcgcaaactggaccgtgccctgcctgatcctatcaaaatcttcgagatcggcccgtgttatcgtaaagagtccgacggtaaagaacatctggaggagtttaccatgctgaacttttgccaaatgggttcaggttgtactcgtgagaacctggaaagcatcatcaccgattttctgaaccacctgggcattgacttcaaaattgtgggcgacagctgtatggtgtttggcgacaccctggatgtcatgcacggcgacctggaactgtctagtgccgttgtgggcccaatcccgctggatcgtgagtggggtatcgacaaaccttggatcggtgcgggttttggtctggagcgtctgctgaaagtaaaacacgacttcaagaacatcaaacgtgctgcacgttccgagtcctattacaatggtatttctactaacctgtaa

SEQ ID NO: 68 - protein, 454 aa, synthetic construct
  REGION 1..454: note = Synthetic Construct
MDKKPLNTLISATGLWMSRTGTIHKIKHHEVSRSKIYIEMACGDHLVVNNSRSSRTARALRHHKYRKTCKRCRVSDEDLNKFLTKANEDQTSVKVKVVSAPTRTKKAMPKSVARAPKPLENTEAAQAQPSGSKFSPAIPVSTQESVSVPASVSTSISSISTGATASALVKGNTNPITSMSAPVQASAPALTKSQTDRLEVLLNPKDEISLNSGKPFRELESELLSRRKKDLQQIYAEERENYLGKLEREITRFFVDRGFLEIKSPILIPLEYIERMGIDNDTELSKQIFRVDKNFCLRPMLAPNLANYLRKLDRALPDPIKIFEIGPCYRKESDGKEHLEEFTMLNFCQMGSGCTRENLESIITDFLNHLGIDFKIVGDSCMVFGDTLDVMHGDLELSSAVVGPIPLDREWGIDKPWIGAGFGLERLLKVKHDFKNIKRAARSESYYNGISTNL

SEQ ID NO: 69 - DNA, 1365 bp, synthetic construct
  misc_feature 1..1365: note = PylRS A1 Mutant
  source 1..1365: mol_type = other DNA
atggataaaaaaccgctgaataccctgatctctgctactggtctgtggatgagtcgtaccggaaccattcataaaatcaaacaccacgaggttagccgttcgaaaatctatattgagatggcgtgtggcgatcatctggttgtgaacaatagccgctcttctcgtacagcacgtgcactgcgtcaccacaaatatcgtaaaacctgtaaacgttgccgtgtgtccgatgaggatctgaacaaattcctgacaaaagccaatgaggaccaaacaagcgtgaaagtgaaagtcgttagcgctcctacccgtactaaaaaagcaatgccgaaatccgttgctcgtgcccctaaaccactggaaaacactgaagcagcacaggcacagccgtctggaagcaaattctctccggccattcctgtttctacccaggagtccgtttctgttccagcaagtgtgagcaccagcattagcagtattagcaccggtgccaccgctagcgccctggttaaaggcaataccaatccgattacaagcatgtctgccccggttcaagcatcagctccagcactgacaaaatcccaaaccgatcgtctggaggttctgctgaatccgaaagacgaaatcagcctgaattccggcaaaccgtttcgtgaactggagagcgaactgctgtcacgtcgtaaaaaagacctgcaacaaatctatgccgaagaacgtgagaactatctggggaaactggaacgtgaaatcacccgctttttcgtggatcgtggctttctggagatcaaatccccgattctgattcctctggagtatatcgagcgtatgggcatcgacaatgataccgaactgagcaaacagatcttccgtgtggataaaaacttctgtctgcgccctatgctagcaccaaatctttataactatatgcgcaaactggaccgtgccctgcctgatcctatcaaaatcttcgagatcggcccgtgttatcgtaaagagtccgacggtaaagaacatctggaggagtttaccatgctgaactttggtcaaatgggttcaggttgtactcgtgagaacctggaaagcatcatcaccgattttctgaaccacctgggcattgacttcaaaattgtgggcgacagctgtatggtgtatggcgacaccctggatgtcatgcacggcgacctggaactgtctagtgccgttgttggaccaaggccgctggaccgtgagtggggtatcgacaaaccgtggatcggagcaggattcggtctggaacgcctgctgaaagtgaaacacgacttcaaaaacatcaaacgtgccgcccgttctgaatcgtattataacgggatctctacgaacctgtaa

SEQ ID NO: 70 - protein, 454 aa, synthetic construct
  REGION 1..454: note = Synthetic Construct
MDKKPLNTLISATGLWMSRTGTIHKIKHHEVSRSKIYIEMACGDHLVVNNSRSSRTARALRHHKYRKTCKRCRVSDEDLNKFLTKANEDQTSVKVKVVSAPTRTKKAMPKSVARAPKPLENTEAAQAQPSGSKFSPAIPVSTQESVSVPASVSTSISSISTGATASALVKGNTNPITSMSAPVQASAPALTKSQTDRLEVLLNPKDEISLNSGKPFRELESELLSRRKKDLQQIYAEERENYLGKLEREITRFFVDRGFLEIKSPILIPLEYIERMGIDNDTELSKQIFRVDKNFCLRPMLAPNLYNYMRKLDRALPDPIKIFEIGPCYRKESDGKEHLEEFTMLNFGQMGSGCTRENLESIITDFLNHLGIDFKIVGDSCMVYGDTLDVMHGDLELSSAVVGPRPLDREWGIDKPWIGAGFGLERLLKVKHDFKNIKRAARSESYYNGISTNL

SEQ ID NO: 71 - DNA, 1365 bp, synthetic construct
  misc_feature 1..1365: note = PylRS MMA Mutant
  source 1..1365: mol_type = other DNA
atggataaaaaaccgctgaataccctgatctctgctactggtctgtggatgagtcgtaccggaaccattcataaaatcaaacaccacgaggttagccgttcgaaaatctatattgagatggcgtgtggcgatcatctggttgtgaacaatagccgctcttctcgtacagcacgtgcactgcgtcaccacaaatatcgtaaaacctgtaaacgttgccgtgtgtccgatgaggatctgaacaaattcctgacaaaagccaatgaggaccaaacaagcgtgaaagtgaaagtcgttagcgctcctacccgtactaaaaaagcaatgccgaaatccgttgctcgtgcccctaaaccactggaaaacactgaagcagcacaggcacagccgtctggaagcaaattctctccggccattcctgtttctacccaggagtccgtttctgttccagcaagtgtgagcaccagcattagcagtattagcaccggtgccaccgctagcgccctggttaaaggcaataccaatccgattacaagcatgtctgccccggttcaagcatcagctccagcactgacaaaatcccaaaccgatcgtctggaggttctgctgaatccgaaagacgaaatcagcctgaattccggcaaaccgtttcgtgaactggagagcgaactgctgtcacgtcgtaaaaaagacctgcaacaaatctatgccgaagaacgtgagaactatctggggaaactggaacgtgaaatcacccgctttttcgtggatcgtggctttctggagatcaaatccccgattctgattcctctggagtatatcgagcgtatgggcatcgacaatgataccgaactgagcaaacaaattttccgtgtggataaaaacttctgtctgcgccctatgctggcaccaaatctgatgaactatatgcgcaaactggaccgtgccctgcctgatcctatcaaaatcttcgagatcggcccgtgttatcgtaaagagtccgacggtaaagaacatctggaggagtttaccatgctgaactttgcgcaaatgggttcaggttgtactcgtgagaacctggaaagcatcatcaccgattttctgaaccacctgggcattgacttcaaaattgtgggcgacagctgtatggtgtatggcgacaccctggatgtcatgcacggcgacctggaactgtctagtgccgttgtgggcccaatcccgctggatcgtgagtggggtatcgacaaaccttggatcggtgcgggttttggtctggagcgtctgctgaaagtaaaacacgacttcaagaacatcaaacgtgctgcacgttccgagtcctattacaatggtatttctactaacctgtaa

SEQ ID NO: 72 - protein, 454 aa, synthetic construct
  REGION 1..454: note = Synthetic Construct
MDKKPLNTLISATGLWMSRTGTIHKIKHHEVSRSKIYIEMACGDHLVVNNSRSSRTARALRHHKYRKTCKRCRVSDEDLNKFLTKANEDQTSVKVKVVSAPTRTKKAMPKSVARAPKPLENTEAAQAQPSGSKFSPAIPVSTQESVSVPASVSTSISSISTGATASALVKGNTNPITSMSAPVQASAPALTKSQTDRLEVLLNPKDEISLNSGKPFRELESELLSRRKKDLQQIYAEERENYLGKLEREITRFFVDRGFLEIKSPILIPLEYIERMGIDNDTELSKQIFRVDKNFCLRPMLAPNLMNYMRKLDRALPDPIKIFEIGPCYRKESDGKEHLEEFTMLNFAQMGSGCTRENLESIITDFLNHLGIDFKIVGDSCMVYGDTLDVMHGDLELSSAVVGPIPLDREWGIDKPWIGAGFGLERLLKVKHDFKNIKRAARSESYYNGISTNL

SEQ ID NO: 73 - DNA, 1416 bp, synthetic construct
  misc_feature 1..1416: note = PylRS M.mazeiNES Mutant
  source 1..1416: mol_type = other DNA
atggcgtgcccggtgccgctgcagctgccgccgctggaacgcctgaccctggatgataaaaaaccgctgaataccctgatctctgctactggtctgtggatgagtcgtaccggaaccattcataaaatcaaacaccacgaggttagccgttcgaaaatctatattgagatggcgtgtggcgatcatctggttgtgaacaatagccgctcttctcgtacagcacgtgcactgcgtcaccacaaatatcgtaaaacctgtaaacgttgccgtgtgtccgatgaggatctgaacaaattcctgacaaaagccaatgaggaccaaacaagcgtgaaagtgaaagtcgttagcgctcctacccgtactaaaaaagcaatgccgaaatccgttgctcgtgcccctaaaccactggaaaacactgaagcagcacaggcacagccgtctggaagcaaattctctccggccattcctgtttctacccaggagtccgtttctgttccagcaagtgtgagcaccagcattagcagtattagcaccggtgccaccgctagcgccctggttaaaggcaataccaatccgattacaagcatgtctgccccggttcaagcatcagctccagcactgacaaaatcccaaaccgatcgtctggaggttctgctgaatccgaaagacgaaatcagcctgaattccggcaaaccgtttcgtgaactggagagcgaactgctgtcacgtcgtaaaaaagacctgcaacaaatctatgccgaagaacgtgagaactatctggggaaactggaacgtgaaatcacccgctttttcgtggatcgtggctttctggagatcaaatccccgattctgattcctctggagtatatcgagcgtatgggcatcgacaatgataccgaactgagcaaacaaattttccgtgtggataaaaacttctgtctgcgccctatgctggcaccaaatctgtataactatctgcgcaaactggaccgtgccctgcctgatcctatcaaaatcttcgagatcggcccgtgttatcgtaaagagtccgacggtaaagaacatctggaggagtttaccatgctgaacttttgccaaatgggttcaggttgtactcgtgagaacctggaaagcatcatcaccgattttctgaaccacctgggcattgacttcaaaattgtgggcgacagctgtatggtgtatggcgacaccctggatgtcatgcacggcgacctggaactgtctagtgccgttgtgggcccaatcccgctggatcgtgagtggggtatcgacaaaccttggatcggtgcgggttttggtctggagcgtctgctgaaagtaaaacacgacttcaagaacatcaaacgtgctgcacgttccgagtcctattacaatggtatttctactaacctgtaa

SEQ ID NO: 74 - protein, 471 aa, synthetic construct
  REGION 1..471: note = Synthetic Construct
MACPVPLQLPPLERLTLDDKKPLNTLISATGLWMSRTGTIHKIKHHEVSRSKIYIEMACGDHLVVNNSRSSRTARALRHHKYRKTCKRCRVSDEDLNKFLTKANEDQTSVKVKVVSAPTRTKKAMPKSVARAPKPLENTEAAQAQPSGSKFSPAIPVSTQESVSVPASVSTSISSISTGATASALVKGNTNPITSMSAPVQASAPALTKSQTDRLEVLLNPKDEISLNSGKPFRELESELLSRRKKDLQQIYAEERENYLGKLEREITRFFVDRGFLEIKSPILIPLEYIERMGIDNDTELSKQIFRVDKNFCLRPMLAPNLYNYLRKLDRALPDPIKIFEIGPCYRKESDGKEHLEEFTMLNFCQMGSGCTRENLESIITDFLNHLGIDFKIVGDSCMVYGDTLDVMHGDLELSSAVVGPIPLDREWGIDKPWIGAGFGLERLLKVKHDFKNIKRAARSESYYNGISTNL

SEQ ID NO: 75 - DNA, 1416 bp, synthetic construct
  misc_feature 1..1416: note = PylRS AF NES Mutant
  source 1..1416: mol_type = other DNA
atggcgtgcccggtgccgctgcagctgccgccgctggaacgcctgaccctggatgataaaaaaccgctgaataccctgatctctgctactggtctgtggatgagtcgtaccggaaccattcataaaatcaaacaccacgaggttagccgttcgaaaatctatattgagatggcgtgtggcgatcatctggttgtgaacaatagccgctcttctcgtacagcacgtgcactgcgtcaccacaaatatcgtaaaacctgtaaacgttgccgtgtgtccgatgaggatctgaacaaattcctgacaaaagccaatgaggaccaaacaagcgtgaaagtgaaagtcgttagcgctcctacccgtactaaaaaagcaatgccgaaatccgttgctcgtgcccctaaaccactggaaaacactgaagcagcacaggcacagccgtctggaagcaaattctctccggccattcctgtttctacccaggagtccgtttctgttccagcaagtgtgagcaccagcattagcagtattagcaccggtgccaccgctagcgccctggttaaaggcaataccaatccgattacaagcatgtctgccccggttcaagcatcagctccagcactgacaaaatcccaaaccgatcgtctggaggttctgctgaatccgaaagacgaaatcagcctgaattccggcaaaccgtttcgtgaactggagagcgaactgctgtcacgtcgtaaaaaagacctgcaacaaatctatgccgaagaacgtgagaactatctggggaaactggaacgtgaaatcacccgctttttcgtggatcgtggctttctggagatcaaatccccgattctgattcctctggagtatatcgagcgtatgggcatcgacaatgataccgaactgagcaaacaaattttccgtgtggataaaaacttctgtctgcgccctatgctagcaccaaatctggctaactatctgcgcaaactggaccgtgccctgcctgatcctatcaaaatcttcgagatcggcccgtgttatcgtaaagagtccgacggtaaagaacatctggaggagtttaccatgctgaacttttgccaaatgggttcaggttgtactcgtgagaacctggaaagcatcatcaccgattttctgaaccacctgggcattgacttcaaaattgtgggcgacagctgtatggtgtttggcgacaccctggatgtcatgcacggcgacctggaactgtctagtgccgttgtgggcccaatcccgctggatcgtgagtggggtatcgacaaaccttggatcggtgcgggttttggtctggagcgtctgctgaaagtaaaacacgacttcaagaacatcaaacgtgctgcacgttccgagtcctattacaatggtatttctactaacctgtaa

SEQ ID NO: 76 - protein, 471 aa, synthetic construct
  REGION 1..471: note = Synthetic Construct
MACPVPLQLPPLERLTLDDKKPLNTLISATGLWMSRTGTIHKIKHHEVSRSKIYIEMACGDHLVVNNSRSSRTARALRHHKYRKTCKRCRVSDEDLNKFLTKANEDQTSVKVKVVSAPTRTKKAMPKSVARAPKPLENTEAAQAQPSGSKFSPAIPVSTQESVSVPASVSTSISSISTGATASALVKGNTNPITSMSAPVQASAPALTKSQTDRLEVLLNPKDEISLNSGKPFRELESELLSRRKKDLQQIYAEERENYLGKLEREITRFFVDRGFLEIKSPILIPLEYIERMGIDNDTELSKQIFRVDKNFCLRPMLAPNLANYLRKLDRALPDPIKIFEIGPCYRKESDGKEHLEEFTMLNFCQMGSGCTRENLESIITDFLNHLGIDFKIVGDSCMVFGDTLDVMHGDLELSSAVVGPIPLDREWGIDKPWIGAGFGLERLLKVKHDFKNIKRAARSESYYNGISTNL

SEQ ID NO: 77 - DNA, 1416 bp, synthetic construct
  misc_feature 1..1416: note = PylRS A1 NES Mutant
  source 1..1416: mol_type = other DNA
atggcgtgcccggtgccgctgcagctgccgccgctggaacgcctgaccctggatgataaaaaaccgctgaataccctgatctctgctactggtctgtggatgagtcgtaccggaaccattcataaaatcaaacaccacgaggttagccgttcgaaaatctatattgagatggcgtgtggcgatcatctggttgtgaacaatagccgctcttctcgtacagcacgtgcactgcgtcaccacaaatatcgtaaaacctgtaaacgttgccgtgtgtccgatgaggatctgaacaaattcctgacaaaagccaatgaggaccaaacaagcgtgaaagtgaaagtcgttagcgctcctacccgtactaaaaaagcaatgccgaaatccgttgctcgtgcccctaaaccactggaaaacactgaagcagcacaggcacagccgtctggaagcaaattctctccggccattcctgtttctacccaggagtccgtttctgttccagcaagtgtgagcaccagcattagcagtattagcaccggtgccaccgctagcgccctggttaaaggcaataccaatccgattacaagcatgtctgccccggttcaagcatcagctccagcactgacaaaatcccaaaccgatcgtctggaggttctgctgaatccgaaagacgaaatcagcctgaattccggcaaaccgtttcgtgaactggagagcgaactgctgtcacgtcgtaaaaaagacctgcaacaaatctatgccgaagaacgtgagaactatctggggaaactggaacgtgaaatcacccgctttttcgtggatcgtggctttctggagatcaaatccccgattctgattcctctggagtatatcgagcgtatgggcatcgacaatgataccgaactgagcaaacagatcttccgtgtggataaaaacttctgtctgcgccctatgctagcaccaaatctttataactatatgcgcaaactggaccgtgccctgcctgatcctatcaaaatcttcgagatcggcccgtgttatcgtaaagagtccgacggtaaagaacatctggaggagtttaccatgctgaactttggtcaaatgggttcaggttgtactcgtgagaacctggaaagcatcatcaccgattttctgaaccacctgggcattgacttcaaaattgtgggcgacagctgtatggtgtatggcgacaccctggatgtcatgcacggcgacctggaactgtctagtgccgttgttggaccaaggccgctggaccgtgagtggggtatcgacaaaccgtggatcggagcaggattcggtctggaacgcctgctgaaagtgaaacacgacttcaaaaacatcaaacgtgccgcccgttctgaatcgtattataacgggatctctacgaacctgtaa

SEQ ID NO: 78 - protein, 471 aa, synthetic construct
  REGION 1..471: note = Synthetic Construct
MACPVPLQLPPLERLTLDDKKPLNTLISATGLWMSRTGTIHKIKHHEVSRSKIYIEMACGDHLVVNNSRSSRTARALRHHKYRKTCKRCRVSDEDLNKFLTKANEDQTSVKVKVVSAPTRTKKAMPKSVARAPKPLENTEAAQAQPSGSKFSPAIPVSTQESVSVPASVSTSISSISTGATASALVKGNTNPITSMSAPVQASAPALTKSQTDRLEVLLNPKDEISLNSGKPFRELESELLSRRKKDLQQIYAEERENYLGKLEREITRFFVDRGFLEIKSPILIPLEYIERMGIDNDTELSKQIFRVDKNFCLRPMLAPNLYNYMRKLDRALPDPIKIFEIGPCYRKESDGKEHLEEFTMLNFGQMGSGCTRENLESIITDFLNHLGIDFKIVGDSCMVYGDTLDVMHGDLELSSAVVGPRPLDREWGIDKPWIGAGFGLERLLKVKHDFKNIKRAARSESYYNGISTNL

SEQ ID NO: 79 - DNA, 1416 bp, synthetic construct
  misc_feature 1..1416: note = PylRS MMA NES Mutant
  source 1..1416: mol_type = other DNA
atggcgtgcccggtgccgctgcagctgccgccgctggaacgcctgaccctggatgataaaaaaccgctgaataccctgatctctgctactggtctgtggatgagtcgtaccggaaccattcataaaatcaaacaccacgaggttagccgttcgaaaatctatattgagatggcgtgtggcgatcatctggttgtgaacaatagccgctcttctcgtacagcacgtgcactgcgtcaccacaaatatcgtaaaacctgtaaacgttgccgtgtgtccgatgaggatctgaacaaattcctgacaaaagccaatgaggaccaaacaagcgtgaaagtgaaagtcgttagcgctcctacccgtactaaaaaagcaatgccgaaatccgttgctcgtgcccctaaaccactggaaaacactgaagcagcacaggcacagccgtctggaagcaaattctctccggccattcctgtttctacccaggagtccgtttctgttccagcaagtgtgagcaccagcattagcagtattagcaccggtgccaccgctagcgccctggttaaaggcaataccaatccgattacaagcatgtctgccccggttcaagcatcagctccagcactgacaaaatcccaaaccgatcgtctggaggttctgctgaatccgaaagacgaaatcagcctgaattccggcaaaccgtttcgtgaactggagagcgaactgctgtcacgtcgtaaaaaagacctgcaacaaatctatgccgaagaacgtgagaactatctggggaaactggaacgtgaaatcacccgctttttcgtggatcgtggctttctggagatcaaatccccgattctgattcctctggagtatatcgagcgtatgggcatcgacaatgataccgaactgagcaaacaaattttccgtgtggataaaaacttctgtctgcgccctatgctggcaccaaatctgatgaactatatgcgcaaactggaccgtgccctgcctgatcctatcaaaatcttcgagatcggcccgtgttatcgtaaagagtccgacggtaaagaacatctggaggagtttaccatgctgaactttgcgcaaatgggttcaggttgtactcgtgagaacctggaaagcatcatcaccgattttctgaaccacctgggcattgacttcaaaattgtgggcgacagctgtatggtgtatggcgacaccctggatgtcatgcacggcgacctggaactgtctagtgccgttgtgggcccaatcccgctggatcgtgagtggggtatcgacaaaccttggatcggtgcgggttttggtctggagcgtctgctgaaagtaaaacacgacttcaagaacatcaaacgtgctgcacgttccgagtcctattacaatggtatttctactaacctgtaa

SEQ ID NO: 80 - protein, 471 aa, synthetic construct
  REGION 1..471: note = Synthetic Construct
MACPVPLQLPPLERLTLDDKKPLNTLISATGLWMSRTGTIHKIKHHEVSRSKIYIEMACGDHLVVNNSRSSRTARALRHHKYRKTCKRCRVSDEDLNKFLTKANEDQTSVKVKVVSAPTRTKKAMPKSVARAPKPLENTEAAQAQPSGSKFSPAIPVSTQESVSVPASVSTSISSISTGATASALVKGNTNPITSMSAPVQASAPALTKSQTDRLEVLLNPKDEISLNSGKPFRELESELLSRRKKDLQQIYAEERENYLGKLEREITRFFVDRGFLEIKSPILIPLEYIERMGIDNDTELSKQIFRVDKNFCLRPMLAPNLMNYMRKLDRALPDPIKIFEIGPCYRKESDGKEHLEEFTMLNFAQMGSGCTRENLESIITDFLNHLGIDFKIVGDSCMVYGDTLDVMHGDLELSSAVVGPIPLDREWGIDKPWIGAGFGLERLLKVKHDFKNIKRAARSESYYNGISTNL

SEQ ID NO: 81 - DNA, 1365 bp, synthetic construct
  misc_feature 1..1365: note = PylRS B11 Mutant
  source 1..1365: mol_type = other DNA
atggataaaaaaccgctgaataccctgatctctgctactggtctgtggatgagtcgtaccggaaccattcataaaatcaaacaccacgaggttagccgttcgaaaatctatattgagatggcgtgtggcgatcatctggttgtgaacaatagccgctcttctcgtacagcacgtgcactgcgtcaccacaaatatcgtaaaacctgtaaacgttgccgtgtgtccgatgaggatctgaacaaattcctgacaaaagccaatgaggaccaaacaagcgtgaaagtgaaagtcgttagcgctcctacccgtactaaaaaagcaatgccgaaatccgttgctcgtgcccctaaaccactggaaaacactgaagcagcacaggcacagccgtctggaagcaaattctctccggccattcctgtttctacccaggagtccgtttctgttccagcaagtgtgagcaccagcattagcagtattagcaccggtgccaccgctagcgccctggttaaaggcaataccaatccgattacaagcatgtctgccccggttcaagcatcagctccagcactgacaaaatcccaaaccgatcgtctggaggttctgctgaatccgaaagacgaaatcagcctgaattccggcaaaccgtttcgtgaactggagagcgaactgctgtcacgtcgtaaaaaagacctgcaacaaatctatgccgaagaacgtgagaactatctggggaaactggaacgtgaaatcacccgctttttcgtggatcgtggctttctggagatcaaatccccgattctgattcctctggagtatatcgagcgtatgggcatcgacaatgataccgaactgagcaaacagatcttccgtgtggataaaaacttctgtctgcgccctatgctagcaccaaatttgtataactatgttcgcaaactggaccgtgccctgcctgatcctatcaaaatcttcgagatcggcccgtgttatcgtaaagagtccgacggtaaagaacatctggaggagtttaccatgctgaactttggtcaaatgggttcaggttgtactcgtgagaacctggaaagcatcatcaccgattttctgaaccacctgggcattgacttcaaaattgtgggcgacagctgtatggtgtatggcgacaccctggatgtcatgcacggcgacctggaactgtctagtgccgttgttggaccaaatccgctggaccgtgagtggggtatcgacaaaccgtggatcggagcaggattcggtctggaacgcctgctgaaagtgaaacacgacttcaaaaacatcaaacgtgccgcccgttctgaatcgtattataacgggatctctacgaacctgtaa

SEQ ID NO: 82 - protein, 454 aa, synthetic construct
  REGION 1..454: note = Synthetic Construct
MDKKPLNTLISATGLWMSRTGTIHKIKHHEVSRSKIYIEMACGDHLVVNNSRSSRTARALRHHKYRKTCKRCRVSDEDLNKFLTKANEDQTSVKVKVVSAPTRTKKAMPKSVARAPKPLENTEAAQAQPSGSKFSPAIPVSTQESVSVPASVSTSISSISTGATASALVKGNTNPITSMSAPVQASAPALTKSQTDRLEVLLNPKDEISLNSGKPFRELESELLSRRKKDLQQIYAEERENYLGKLEREITRFFVDRGFLEIKSPILIPLEYIERMGIDNDTELSKQIFRVDKNFCLRPMLAPNLYNYVRKLDRALPDPIKIFEIGPCYRKESDGKEHLEEFTMLNFGQMGSGCTRENLESIITDFLNHLGIDFKIVGDSCMVYGDTLDVMHGDLELSSAVVGPNPLDREWGIDKPWIGAGFGLERLLKVKHDFKNIKRAARSESYYNGISTNL

SEQ ID NO: 83 - DNA, 1365 bp, synthetic construct
  misc_feature 1..1365: note = PylRS C11 Mutant
  source 1..1365: mol_type = other DNA
atggataaaaaaccgctgaataccctgatctctgctactggtctgtggatgagtcgtaccggaaccattcataaaatcaaacaccacgaggttagccgttcgaaaatctatattgagatggcgtgtggcgatcatctggttgtgaacaatagccgctcttctcgtacagcacgtgcactgcgtcaccacaaatatcgtaaaacctgtaaacgttgccgtgtgtccgatgaggatctgaacaaattcctgacaaaagccaatgaggaccaaacaagcgtgaaagtgaaagtcgttagcgctcctacccgtactaaaaaagcaatgccgaaatccgttgctcgtgcccctaaaccactggaaaacactgaagcagcacaggcacagccgtctggaagcaaattctctccggccattcctgtttctacccaggagtccgtttctgttccagcaagtgtgagcaccagcattagcagtattagcaccggtgccaccgctagcgccctggttaaaggcaataccaatccgattacaagcatgtctgccccggttcaagcatcagctccagcactgacaaaatcccaaaccgatcgtctggaggttctgctgaatccgaaagacgaaatcagcctgaattccggcaaaccgtttcgtgaactggagagcgaactgctgtcacgtcgtaaaaaagacctgcaacaaatctatgccgaagaacgtgagaactatctggggaaactggaacgtgaaatcacccgctttttcgtggatcgtggctttctggagatcaaatccccgattctgattcctctggagtatatcgagcgtatgggcatcgacaatgataccgaactgagcaaacagatcttccgtgtggataaaaacttctgtctgcgccctatgctagcaccaaatttgtataactattgtcgcaaactggaccgtgccctgcctgatcctatcaaaatcttcgagatcggcccgtgttatcgtaaagagtccgacggtaaagaacatctggaggagtttaccatgctgaactttgggcaaatgggttcaggttgtactcgtgagaacctggaaagcatcatcaccgattttctgaaccacctgggcattgacttcaaaattgtgggcgacagctgtatggtgtatggcgacaccctggatgtcatgcacggcgacctggaactgtctagtgccgttgttggaccaattccgctggaccgtgagtggggtatcgacaaaccgtggatcggagcaggattcggtctggaacgcctgctgaaagtgaaacacgacttcaaaaacatcaaacgtgccgcccgttctgaatcgtattataacgggatctctacgaacctgtaa

SEQ ID NO: 84 - protein, 454 aa, synthetic construct
  REGION 1..454: note = Synthetic Construct
MDKKPLNTLISATGLWMSRTGTIHKIKHHEVSRSKIYIEMACGDHLVVNNSRSSRTARALRHHKYRKTCKRCRVSDEDLNKFLTKANEDQTSVKVKVVSAPTRTKKAMPKSVARAPKPLENTEAAQAQPSGSKFSPAIPVSTQESVSVPASVSTSISSISTGATASALVKGNTNPITSMSAPVQASAPALTKSQTDRLEVLLNPKDEISLNSGKPFRELESELLSRRKKDLQQIYAEERENYLGKLEREITRFFVDRGFLEIKSPILIPLEYIERMGIDNDTELSKQIFRVDKNFCLRPMLAPNLYNYCRKLDRALPDPIKIFEIGPCYRKESDGKEHLEEFTMLNFGQMGSGCTRENLESIITDFLNHLGIDFKIVGDSCMVYGDTLDVMHGDLELSSAVVGPIPLDREWGIDKPWIGAGFGLERLLKVKHDFKNIKRAARSESYYNGISTNL

SEQ ID NO: 85 - DNA, 1365 bp, synthetic construct
  misc_feature 1..1365: note = PylRS G3 Mutant
  source 1..1365: mol_type = other DNA
atggataaaaaaccgctgaataccctgatctctgctactggtctgtggatgagtcgtaccggaaccattcataaaatcaaacaccacgaggttagccgttcgaaaatctatattgagatggcgtgtggcgatcatctggttgtgaacaatagccgctcttctcgtacagcacgtgcactgcgtcaccacaaatatcgtaaaacctgtaaacgttgccgtgtgtccgatgaggatctgaacaaattcctgacaaaagccaatgaggaccaaacaagcgtgaaagtgaaagtcgttagcgctcctacccgtactaaaaaagcaatgccgaaatccgttgctcgtgcccctaaaccactggaaaacactgaagcagcacaggcacagccgtctggaagcaaattctctccggccattcctgtttctacccaggagtccgtttctgttccagcaagtgtgagcaccagcattagcagtattagcaccggtgccaccgctagcgccctggttaaaggcaataccaatccgattacaagcatgtctgccccggttcaagcatcagctccagcactgacaaaatcccaaaccgatcgtctggaggttctgctgaatccgaaagacgaaatcagcctgaattccggcaaaccgtttcgtgaactggagagcgaactgctgtcacgtcgtaaaaaagacctgcaacaaatctatgccgaagaacgtgagaactatctggggaaactggaacgtgaaatcacccgctttttcgtggatcgtggctttctggagatcaaatccccgattctgattcctctggagtatatcgagcgtatgggcatcgacaatgataccgaactgagcaaacagatcttccgtgtggataaaaacttctgtctgcgccctatgctagcaccaaatttgtataactatgtgcgcaaactggaccgtgccctgcctgatcctatcaaaatcttcgagatcggcccgtgttatcgtaaagagtccgacggtaaagaacatctggaggagtttaccatgctgaactttgctcaaatgggttcaggttgtactcgtgagaacctggaaagcatcatcaccgattttctgaaccacctgggcattgacttcaaaattgtgggcgacagctgtatggtgtatggcgacaccctggatgtcatgcacggcgacctggaactgtctagtgccgttgttggaccacgtccgctggaccgtgagtggggtatcgacaaaccgtggatcggagcaggattcggtctggaacgcctgctgaaagtgaaacacgacttcaaaaacatcaaacgtgccgcccgttctgaatcgtattataacgggatctctacgaacctgtaa

SEQ ID NO: 86 - protein, 454 aa, synthetic construct
  REGION 1..454: note = Synthetic Construct
MDKKPLNTLISATGLWMSRTGTIHKIKHHEVSRSKIYIEMACGDHLVVNNSRSSRTARALRHHKYRKTCKRCRVSDEDLNKFLTKANEDQTSVKVKVVSAPTRTKKAMPKSVARAPKPLENTEAAQAQPSGSKFSPAIPVSTQESVSVPASVSTSISSISTGATASALVKGNTNPITSMSAPVQASAPALTKSQTDRLEVLLNPKDEISLNSGKPFRELESELLSRRKKDLQQIYAEERENYLGKLEREITRFFVDRGFLEIKSPILIPLEYIERMGIDNDTELSKQIFRVDKNFCLRPMLAPNLYNYVRKLDRALPDPIKIFEIGPCYRKESDGKEHLEEFTMLNFAQMGSGCTRENLESIITDFLNHLGIDFKIVGDSCMVYGDTLDVMHGDLELSSAVVGPRPLDREWGIDKPWIGAGFGLERLLKVKHDFKNIKRAARSESYYNGISTNL

SEQ ID NO: 87 - DNA, 1365 bp, synthetic construct
  misc_feature 1..1365: note = PylRS H12 mutant
  source 1..1365: mol_type = other DNA
atggataaaaaaccgctgaataccctgatctctgctactggtctgtggatgagtcgtaccggaaccattcataaaatcaaacaccacgaggttagccgttcgaaaatctatattgagatggcgtgtggcgatcatctggttgtgaacaatagccgctcttctcgtacagcacgtgcactgcgtcaccacaaatatcgtaaaacctgtaaacgttgccgtgtgtccgatgaggatctgaacaaattcctgacaaaagccaatgaggaccaaacaagcgtgaaagtgaaagtcgttagcgctcctacccgtactaaaaaagcaatgccgaaatccgttgctcgtgcccctaaaccactggaaaacactgaagcagcacaggcacagccgtctggaagcaaattctctccggccattcctgtttctacccaggagtccgtttctgttccagcaagtgtgagcaccagcattagcagtattagcaccggtgccaccgctagcgccctggttaaaggcaataccaatccgattacaagcatgtctgccccggttcaagcatcagctccagcactgacaaaatcccaaaccgatcgtctggaggttctgctgaatccgaaagacgaaatcagcctgaattccggcaaaccgtttcgtgaactggagagcgaactgctgtcacgtcgtaaaaaagacctgcaacaaatctatgccgaagaacgtgagaactatctggggaaactggaacgtgaaatcacccgctttttcgtggatcgtggctttctggagatcaaatccccgattctgattcctctggagtatatcgagcgtatgggcatcgacaatgataccgaactgagcaaacagatcttccgtgtggataaaaacttctgtctgcgccctatgctagcaccaaatttgtataactatgttcgcaaactggaccgtgccctgcctgatcctatcaaaatcttcgagatcggcccgtgttatcgtaaagagtccgacggtaaagaacatctggaggagtttaccatgctgaactttggtcaaatgggttcaggttgtactcgtgagaacctggaaagcatcatcaccgattttctgaaccacctgggcattgacttcaaaattgtgggcgacagctgtatggtgtatggcgacaccctggatgtcatgcacggcgacctggaactgtctagtgccgttgttggaccacggccgctggaccgtgagtggggtatcgacaaaccgtggatcggagcaggattcggtctggaacgcctgctgaaagtgaaacacgacttcaaaaacatcaaacgtgccgcccgttctgaatcgtattataacgggatctctacgaacctgtaa

SEQ ID NO: 88 - protein, 454 aa, synthetic construct
  REGION 1..454: note = Synthetic Construct
MDKKPLNTLISATGLWMSRTGTIHKIKHHEVSRSKIYIEMACGDHLVVNNSRSSRTARALRHHKYRKTCKRCRVSDEDLNKFLTKANEDQTSVKVKVVSAPTRTKKAMPKSVARAPKPLENTEAAQAQPSGSKFSPAIPVSTQESVSVPASVSTSISSISTGATASALVKGNTNPITSMSAPVQASAPALTKSQTDRLEVLLNPKDEISLNSGKPFRELESELLSRRKKDLQQIYAEERENYLGKLEREITRFFVDRGFLEIKSPILIPLEYIERMGIDNDTELSKQIFRVDKNFCLRPMLAPNLYNYVRKLDRALPDPIKIFEIGPCYRKESDGKEHLEEFTMLNFGQMGSGCTRENLESIITDFLNHLGIDFKIVGDSCMVYGDTLDVMHGDLELSSAVVGPRPLDREWGIDKPWIGAGFGLERLLKVKHDFKNIKRAARSESYYNGISTNL

SEQ ID NO: 89 - DNA, 1416 bp, synthetic construct
  misc_feature 1..1416: note = PylRS B11 NES Mutant
  source 1..1416: mol_type = other DNA
atggcgtgcccggtgccgctgcagctgccgccgctggaacgcctgaccctggatgataaaaaaccgctgaataccctgatctctgctactggtctgtggatgagtcgtaccggaaccattcataaaatcaaacaccacgaggttagccgttcgaaaatctatattgagatggcgtgtggcgatcatctggttgtgaacaatagccgctcttctcgtacagcacgtgcactgcgtcaccacaaatatcgtaaaacctgtaaacgttgccgtgtgtccgatgaggatctgaacaaattcctgacaaaagccaatgaggaccaaacaagcgtgaaagtgaaagtcgttagcgctcctacccgtactaaaaaagcaatgccgaaatccgttgctcgtgcccctaaaccactggaaaacactgaagcagcacaggcacagccgtctggaagcaaattctctccggccattcctgtttctacccaggagtccgtttctgttccagcaagtgtgagcaccagcattagcagtattagcaccggtgccaccgctagcgccctggttaaaggcaataccaatccgattacaagcatgtctgccccggttcaagcatcagctccagcactgacaaaatcccaaaccgatcgtctggaggttctgctgaatccgaaagacgaaatcagcctgaattccggcaaaccgtttcgtgaactggagagcgaactgctgtcacgtcgtaaaaaagacctgcaacaaatctatgccgaagaacgtgagaactatctggggaaactggaacgtgaaatcacccgctttttcgtggatcgtggctttctggagatcaaatccccgattctgattcctctggagtatatcgagcgtatgggcatcgacaatgataccgaactgagcaaacagatcttccgtgtggataaaaacttctgtctgcgccctatgctagcaccaaatttgtataactatgttcgcaaactggaccgtgccctgcctgatcctatcaaaatcttcgagatcggcccgtgttatcgtaaagagtccgacggtaaagaacatctggaggagtttaccatgctgaactttggtcaaatgggttcaggttgtactcgtgagaacctggaaagcatcatcaccgattttctgaaccacctgggcattgacttcaaaattgtgggcgacagctgtatggtgtatggcgacaccctggatgtcatgcacggcgacctggaactgtctagtgccgttgttggaccaaatccgctggaccgtgagtggggtatcgacaaaccgtggatcggagcaggattcggtctggaacgcctgctgaaagtgaaacacgacttcaaaaacatcaaacgtgccgcccgttctgaatcgtattataacgggatctctacgaacctgtaa

SEQ ID NO: 90 - protein, 471 aa, synthetic construct
  REGION 1..471: note = Synthetic Construct
MACPVPLQLPPLERLTLDDKKPLNTLISATGLWMSRTGTIHKIKHHEVSRSKIYIEMACGDHLVVNNSRSSRTARALRHHKYRKTCKRCRVSDEDLNKFLTKANEDQTSVKVKVVSAPTRTKKAMPKSVARAPKPLENTEAAQAQPSGSKFSPAIPVSTQESVSVPASVSTSISSISTGATASALVKGNTNPITSMSAPVQASAPALTKSQTDRLEVLLNPKDEISLNSGKPFRELESELLSRRKKDLQQIYAEERENYLGKLEREITRFFVDRGFLEIKSPILIPLEYIERMGIDNDTELSKQIFRVDKNFCLRPMLAPNLYNYVRKLDRALPDPIKIFEIGPCYRKESDGKEHLEEFTMLNFGQMGSGCTRENLESIITDFLNHLGIDFKIVGDSCMVYGDTLDVMHGDLELSSAVVGPNPLDREWGIDKPWIGAGFGLERLLKVKHDFKNIKRAARSESYYNGISTNL

SEQ ID NO: 91 - DNA, 1416 bp, synthetic construct
  misc_feature 1..1416: note = PylRS C11 NES Mutant
  source 1..1416: mol_type = other DNA
atggcgtgcccggtgccgctgcagctgccgccgctggaacgcctgaccctggatgataaaaaaccgctgaataccctgatctctgctactggtctgtggatgagtcgtaccggaaccattcataaaatcaaacaccacgaggttagccgttcgaaaatctatattgagatggcgtgtggcgatcatctggttgtgaacaatagccgctcttctcgtacagcacgtgcactgcgtcaccacaaatatcgtaaaacctgtaaacgttgccgtgtgtccgatgaggatctgaacaaattcctgacaaaagccaatgaggaccaaacaagcgtgaaagtgaaagtcgttagcgctcctacccgtactaaaaaagcaatgccgaaatccgttgctcgtgcccctaaaccactggaaaacactgaagcagcacaggcacagccgtctggaagcaaattctctccggccattcctgtttctacccaggagtccgtttctgttccagcaagtgtgagcaccagcattagcagtattagcaccggtgccaccgctagcgccctggttaaaggcaataccaatccgattacaagcatgtctgccccggttcaagcatcagctccagcactgacaaaatcccaaaccgatcgtctggaggttctgctgaatccgaaagacgaaatcagcctgaattccggcaaaccgtttcgtgaactggagagcgaactgctgtcacgtcgtaaaaaagacctgcaacaaatctatgccgaagaacgtgagaactatctggggaaactggaacgtgaaatcacccgctttttcgtggatcgtggctttctggagatcaaatccccgattctgattcctctggagtatatcgagcgtatgggcatcgacaatgataccgaactgagcaaacagatcttccgtgtggataaaaacttctgtctgcgccctatgctagcaccaaatttgtataactattgtcgcaaactggaccgtgccctgcctgatcctatcaaaatcttcgagatcggcccgtgttatcgtaaagagtccgacggtaaagaacatctggaggagtttaccatgctgaactttgggcaaatgggttcaggttgtactcgtgagaacctggaaagcatcatcaccgattttctgaaccacctgggcattgacttcaaaattgtgggcgacagctgtatggtgtatggcgacaccctggatgtcatgcacggcgacctggaactgtctagtgccgttgttggaccaattccgctggaccgtgagtggggtatcgacaaaccgtggatcggagcaggattcggtctggaacgcctgctgaaagtgaaacacgacttcaaaaacatcaaacgtgccgcccgttctgaatcgtattataacgggatctctacgaacctgtaa

SEQ ID NO: 92 - protein, 471 aa, synthetic construct
  REGION 1..471: note = Synthetic Construct
MACPVPLQLPPLERLTLDDKKPLNTLISATGLWMSRTGTIHKIKHHEVSRSKIYIEMACGDHLVVNNSRSSRTARALRHHKYRKTCKRCRVSDEDLNKFLTKANEDQTSVKVKVVSAPTRTKKAMPKSVARAPKPLENTEAAQAQPSGSKFSPAIPVSTQESVSVPASVSTSISSISTGATASALVKGNTNPITSMSAPVQASAPALTKSQTDRLEVLLNPKDEISLNSGKPFRELESELLSRRKKDLQQIYAEERENYLGKLEREITRFFVDRGFLEIKSPILIPLEYIERMGIDNDTELSKQIFRVDKNFCLRPMLAPNLYNYCRKLDRALPDPIKIFEIGPCYRKESDGKEHLEEFTMLNFGQMGSGCTRENLESIITDFLNHLGIDFKIVGDSCMVYGDTLDVMHGDLELSSAVVGPIPLDREWGIDKPWIGAGFGLERLLKVKHDFKNIKRAARSESYYNGISTNL

SEQ ID NO: 93 - DNA, 1416 bp, synthetic construct
  misc_feature 1..1416: note = PlyRS G3 NES Mutant
  source 1..1416: mol_type = other DNA
atggcgtgcccggtgccgctgcagctgccgccgctggaacgcctgaccctggatgataaaaaaccgctgaataccctgatctctgctactggtctgtggatgagtcgtaccggaaccattcataaaatcaaacaccacgaggttagccgttcgaaaatctatattgagatggcgtgtggcgatcatctggttgtgaacaatagccgctcttctcgtacagcacgtgcactgcgtcaccacaaatatcgtaaaacctgtaaacgttgccgtgtgtccgatgaggatctgaacaaattcctgacaaaagccaatgaggaccaaacaagcgtgaaagtgaaagtcgttagcgctcctacccgtactaaaaaagcaatgccgaaatccgttgctcgtgcccctaaaccactggaaaacactgaagcagcacaggcacagccgtctggaagcaaattctctccggccattcctgtttctacccaggagtccgtttctgttccagcaagtgtgagcaccagcattagcagtattagcaccggtgccaccgctagcgccctggttaaaggcaataccaatccgattacaagcatgtctgccccggttcaagcatcagctccagcactgacaaaatcccaaaccgatcgtctggaggttctgctgaatccgaaagacgaaatcagcctgaattccggcaaaccgtttcgtgaactggagagcgaactgctgtcacgtcgtaaaaaagacctgcaacaaatctatgccgaagaacgtgagaactatctggggaaactggaacgtgaaatcacccgctttttcgtggatcgtggctttctggagatcaaatccccgattctgattcctctggagtatatcgagcgtatgggcatcgacaatgataccgaactgagcaaacagatcttccgtgtggataaaaacttctgtctgcgccctatgctagcaccaaatttgtataactatgtgcgcaaactggaccgtgccctgcctgatcctatcaaaatcttcgagatcggcccgtgttatcgtaaagagtccgacggtaaagaacatctggaggagtttaccatgctgaactttgctcaaatgggttcaggttgtactcgtgagaacctggaaagcatcatcaccgattttctgaaccacctgggcattgacttcaaaattgtgggcgacagctgtatggtgtatggcgacaccctggatgtcatgcacggcgacctggaactgtctagtgccgttgttggaccacgtccgctggaccgtgagtggggtatcgacaaaccgtggatcggagcaggattcggtctggaacgcctgctgaaagtgaaacacgacttcaaaaacatcaaacgtgccgcccgttctgaatcgtattataacgggatctctacgaacctgtaa

SEQ ID NO: 94 - protein, 471 aa, synthetic construct
  REGION 1..471: note = Synthetic Construct
MACPVPLQLPPLERLTLDDKKPLNTLISATGLWMSRTGTIHKIKHHEVSRSKIYIEMACGDHLVVNNSRSSRTARALRHHKYRKTCKRCRVSDEDLNKFLTKANEDQTSVKVKVVSAPTRTKKAMPKSVARAPKPLENTEAAQAQPSGSKFSPAIPVSTQESVSVPASVSTSISSISTGATASALVKGNTNPITSMSAPVQASAPALTKSQTDRLEVLLNPKDEISLNSGKPFRELESELLSRRKKDLQQIYAEERENYLGKLEREITRFFVDRGFLEIKSPILIPLEYIERMGIDNDTELSKQIFRVDKNFCLRPMLAPNLYNYVRKLDRALPDPIKIFEIGPCYRKESDGKEHLEEFTMLNFAQMGSGCTRENLESIITDFLNHLGIDFKIVGDSCMVYGDTLDVMHGDLELSSAVVGPRPLDREWGIDKPWIGAGFGLERLLKVKHDFKNIKRAARSESYYNGISTNL

SEQ ID NO: 95 - DNA, 1416 bp, synthetic construct
  misc_feature 1..1416: note = PylRS H12 NES Mutant
  source 1..1416: mol_type = other DNA
atggcgtgcccggtgccgctgcagctgccgccgctggaacgcctgaccctggatgataaaaaaccgctgaataccctgatctctgctactggtctgtggatgagtcgtaccggaaccattcataaaatcaaacaccacgaggttagccgttcgaaaatctatattgagatggcgtgtggcgatcatctggttgtgaacaatagccgctcttctcgtacagcacgtgcactgcgtcaccacaaatatcgtaaaacctgtaaacgttgccgtgtgtccgatgaggatctgaacaaattcctgacaaaagccaatgaggaccaaacaagcgtgaaagtgaaagtcgttagcgctcctacccgtactaaaaaagcaatgccgaaatccgttgctcgtgcccctaaaccactggaaaacactgaagcagcacaggcacagccgtctggaagcaaattctctccggccattcctgtttctacccaggagtccgtttctgttccagcaagtgtgagcaccagcattagcagtattagcaccggtgccaccgctagcgccctggttaaaggcaataccaatccgattacaagcatgtctgccccggttcaagcatcagctccagcactgacaaaatcccaaaccgatcgtctggaggttctgctgaatccgaaagacgaaatcagcctgaattccggcaaaccgtttcgtgaactggagagcgaactgctgtcacgtcgtaaaaaagacctgcaacaaatctatgccgaagaacgtgagaactatctggggaaactggaacgtgaaatcacccgctttttcgtggatcgtggctttctggagatcaaatccccgattctgattcctctggagtatatcgagcgtatgggcatcgacaatgataccgaactgagcaaacagatcttccgtgtggataaaaacttctgtctgcgccctatgctagcaccaaatttgtataactatgttcgcaaactggaccgtgccctgcctgatcctatcaaaatcttcgagatcggcccgtgttatcgtaaagagtccgacggtaaagaacatctggaggagtttaccatgctgaactttggtcaaatgggttcaggttgtactcgtgagaacctggaaagcatcatcaccgattttctgaaccacctgggcattgacttcaaaattgtgggcgacagctgtatggtgtatggcgacaccctggatgtcatgcacggcgacctggaactgtctagtgccgttgttggaccacggccgctggaccgtgagtggggtatcgacaaaccgtggatcggagcaggattcggtctggaacgcctgctgaaagtgaaacacgacttcaaaaacatcaaacgtgccgcccgttctgaatcgtattataacgggatctctacgaacctgtaa

SEQ ID NO: 96 - protein, 471 aa, synthetic construct
  REGION 1..471: note = Synthetic Construct
MACPVPLQLPPLERLTLDDKKPLNTLISATGLWMSRTGTIHKIKHHEVSRSKIYIEMACGDHLVVNNSRSSRTARALRHHKYRKTCKRCRVSDEDLNKFLTKANEDQTSVKVKVVSAPTRTKKAMPKSVARAPKPLENTEAAQAQPSGSKFSPAIPVSTQESVSVPASVSTSISSISTGATASALVKGNTNPITSMSAPVQASAPALTKSQTDRLEVLLNPKDEISLNSGKPFRELESELLSRRKKDLQQIYAEERENYLGKLEREITRFFVDRGFLEIKSPILIPLEYIERMGIDNDTELSKQIFRVDKNFCLRPMLAPNLYNYVRKLDRALPDPIKIFEIGPCYRKESDGKEHLEEFTMLNFGQMGSGCTRENLESIITDFLNHLGIDFKIVGDSCMVYGDTLDVMHGDLELSSAVVGPRPLDREWGIDKPWIGAGFGLERLLKVKHDFKNIKRAARSESYYNGISTNL

SEQ ID NO: 97 - DNA, 5978 bp, synthetic construct
  misc_feature 1..5978: note = Plasmid
  source 1..5978: mol_type = other DNA
caaavcttctcatagacatacatgtccaattccatcaaavctcctaactgaataactgccttvcggttaatttttacttatttcggvataatgaaggtaactaaacggtcvtatacattattaacatactaacacccattaacttattctttaacattcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatcactagaagctttattgcggtagtttatcacagttaaattgctaacgcagtcagtgcttctgacacaacagtctcgaacttaagctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagccaccatgggccgcctggaaagcaccccgccgaaaaaaaaacgcaaagtggaagatagcgcgagcgcggaaggatccgtcgccaggcagcctgacctcttgacctgcgacgatgagccgatccatatccccggtgccatccaaccgcatggactgctgctcgccctcgccgccgacatgacgatcgttgccggcagcgacaaccttcccgaactcaccggactggcgatcggcgccctgatcggccgctctgcggccgatgtcttcgactcggagacgcacaaccgtctgacgatcgccttggccgagcccggggcggccgtcggagcaccgatcactgtcggcttcacgatgcgaaaggacgcaggcttcatcggctcctggcatcgccatgatcagctcatcttcctcgagctcgagcctccccagcgggacgtcgccgagccgcaggcgttcttccgccgcaccaacagcgccatccgccgcctgcaggccgccgaaaccttggaaagcgcctgcgccgccgcggcgcaagaggtgcggaagattaccggcttcgatcgggtgatgatctatcgcttcgcctccgacttcagcggcgaagtgatcgcagaggatcggtgcgccgaggtcgagtcaaaactaggcctgcactatcctgcctcaaccgtgccggcgcaggcccgtcggctctataccatcaacccggtacggatcattcccgatatcaattatcggccggtgccggtcaccccagacctcaatccggtcaccgggcggccgattgatcttagcttcgccatcctgcgcagcgtctcgcccgtccatctggaattcatgcgcaacataggcatgcacggcacgatgtcgatctcgattttgcgcggcgagcgactgtggggattgatcgtttgccatcaccgaacgccgtactacgtcgatctcgatggccgccaagcctgcgagctagtcgcccaggttctggcctggcagatcggcgtgatggaagagctggcggaagcggcggccaaagaagcggcggccaaagaagcggcggccaaagaagcggcggccaaagcggcggccggtaccgattacaaagatgatgatgataaagtgagcaagggcgaggagctgttcaccggggtggtgcccatcctggtcgagctggacggcgacgtaaacggccacaagttcagcgtgtccggcgagggcgagggcgatgccacctagggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggccaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagcatcaccatcaccatcactgagcggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatcgataaggatccgggctggcgtaatagcgaagaggcccgcaccgatcgcccttcccaacagttgcgcagcctgaatggcgaatggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatggctcgacagatctvctca

SEQ ID NO: 98 - DNA, 7461 bp, synthetic construct
  misc_feature 1..7461: note = Plasmid
  source 1..7461: mol_type = other DNA
gccagatatacgccaatattggccattagccatattattcattggttatatagcataaatcaatattggctattggccattgcatacgttgtatctatatcataatatgtacatttatattggctcatgtccaatatgaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagagtctataggcccacccccttggcttcgttagaacgcggctacaattaatacataaccttatgtatcatacacatacgatttaggtgacactatagaataacatccactttgcctttctctccacaggtgtccactcccaggtccaactgcacggaagcttcaccatggcgtgcccggtgccgctgcagctgccgccgctggaacgcctgaccctggatgataaaaaaccgctgaataccctgatctctgctactggtctgtggatgagtcgtaccggaaccattcataaaatcaaacaccacgaggttagccgttcgaaaatctatattgagatggcgtgtggcgatcatctggttgtgaacaatagccgctcttctcgtacagcacgtgcactgcgtcaccacaaatatcgtaaaacctgtaaacgttgccgtgtgtccgatgaggatctgaacaaattcctgacaaaagccaatgaggaccaaacaagcgtgaaagtgaaagtcgttagcgctcctacccgtactaaaaaagcaatgccgaaatccgttgctcgtgcccctaaaccactggaaaacactgaagcagcacaggcacagccgtctggaagcaaattctctccggccattcctgtttctacccaggagtccgtttctgttccagcaagtgtgagcaccagcattagcagtattagcaccggtgccaccgctagcgccctggttaaaggcaataccaatccgattacaagcatgtctgccccggttcaagcatcagctccagcactgacaaaatcccaaaccgatcgtctggaggttctgctgaatccgaaagacgaaatcagcctgaattccggcaaaccgtttcgtgaactggagagcgaactgctgtcacgtcgtaaaaaagacctgcaacaaatctatgccgaagaacgtgagaactatctggggaaactggaacgtgaaatcacccgctttttcgtggatcgtggctttctggagatcaaatccccgattctgattcctctggagtatatcgagcgtatgggcatcgacaatgataccgaactgagcaaacaaattttccgtgtggataaaaacttctgtctgcgccctatgctagcaccaaatctggctaactatctgcgcaaactggaccgtgccctgcctgatcctatcaaaatcttcgagatcggcccgtgttatcgtaaagagtccgacggtaaagaacatctggaggagtttaccatgctgaacttttgccaaatgggttcaggttgtactcgtgagaacctggaaagcatcatcaccgattttctgaaccacctgggcattgacttcaaaattgtgggcgacagctgtatggtgtttggcgacaccctggatgtcatgcacggcgacctggaactgtctagtgccgttgtgggcccaatcccgctggatcgtgagtggggtatcgacaaaccttggatcggtgcgggttttggtctggagcgtctgctgaaagtaaaacacgacttcaagaacatcaaacgtgctgcacgttccgagtcctattacaatggtatttctactaacctgtaaggatccactagtccagtgtggtggaattctgcagatatccagcacagtggcggccgctcgagtctagaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagcacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaaatggcggaaaccccgggaatctaacccggctgaacggatttagagtccattcgatctacatgatcaggtttccggtgtttcgtcctttccacaagatatataaagccaagaaatcgaaatactttcaagttacggtaagcatatgatagtccattttaaaacataattttaaaactgcaaactacccaagaaattattactttctacgtcacgtattttgtactaatatctttgtgtttacagtcaaattaattctaattatctctctaacagccttgtatcgtatatgcaaatatgaaggaatcatgggaaataggccctcttcctgcccgaccttcgcgatgtacgg

SEQ ID NO: 99 - DNA, 3382 bp, synthetic construct
  misc_feature 1..3382: note = Plasmid
  source 1..3382: mol_type = other DNA
tcagaattggttaattggttgtaacactggcagagcattacgctgacttgacgggacggcggctttgttgaataaatcgaacttttgctgagttgaaggatcctcgggttgtcagcctgtcccgcttataagatcatacgccgttatacgttgtttacgctttgaggaatcccatatggacaaaaaaccgctgaataccctgatctctgctactggtctgtggatgagtcgtaccggaaccattcataaaatcaaacaccacgaggttagccgttcgaaaatctatattgagatggcgtgtggcgatcatctggttgtgaacaatagccgctcttctcgtacagcacgtgcactgcgtcaccacaaatatcgtaaaacctgtaaacgttgccgtgtgtccgatgaggatctgaacaaattcctgacaaaagccaatgaggaccaaacaagcgtgaaagtgaaagtcgttagcgctcctacccgtactaaaaaagcaatgccgaaatccgttgctcgtgcccctaaaccactggaaaacactgaagcagcacaggcacagccgtctggaagcaaattctctccggccattcctgtttctacccaggagtccgtttctgttccagcaagtgtgagcaccagcattagcagtattagcaccggtgccaccgctagcgccctggttaaaggcaataccaatccgattacaagcatgtctgccccggttcaagcatcagctccagcactgacaaaatcccaaaccgatcgtctggaggttctgctgaatccgaaagacgaaatcagcctgaattccggcaaaccgtttcgtgaactggagagcgaactgctgtcacgtcgtaaaaaagacctgcaacaaatctatgccgaagaacgtgagaactatctggggaaactggaacgtgaaatcacccgctttttcgtggatcgtggctttctggagatcaaatccccgattctgattcctctggagtatatcgagcgtatgggcatcgacaatgataccgaactgagcaaacagatcttccgtgtggataaaaacttctgtctgcgccctatgctggcaccaaatctgtataactatctgcgcaaactggaccgtgccctgcctgatcctatcaaaatcttcgagatcggcccgtgttatcgtaaagagtccgacggtaaagaacatctggaggagtttaccatgctgaacttttgccaaatgggttcaggttgtactcgtgagaacctggaaagcatcatcaccgattttctgaaccacctgggcattgacttcaaaattgtgggcgacagctgtatggtgtatggcgacaccctggatgtcatgcacggcgacctggaactgtctagtgccgttgttggaccaattccgctggaccgtgagtggggtatcgacaaaccgtggatcggagcaggattcggtctggaacgcctgctgaaagtgaaacacgacttcaaaaacatcaaacgtgccgcccgttctgaatcgtattataacgggatctctacgaacctgtaactgcagtttcaaacgctaaattgcctgatgcgctacgcttatcaggcctacatgatctctgcaatatattgagtttgcgtgcttttgtaggccggataaggcgttcacgccgcatccggcaagaaacagcaaacaatccaaaacgccgcgttcagcggcgttttttctgcttttcttcgcgaattaattccgcttcgcacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagttgtgtctcaaaatctctgatgttacattgcacaagataaaaatatatcatcatgaacaataaaactgtctgcttacataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaa

SEQ ID NO: 100 - DNA, 10326 bp, synthetic construct
  misc_feature 1..10326: note = Plasmid
  source 1..10326: mol_type = other DNA
gcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccttaataagatgatcttcttgagatcgttttggtctgcgcgtaatctcttgctctgaaaacgaaaaaaccgccttgcagggcggtttttcgaaggttctctgagctaccaactctttgaaccgaggtaactggcttggaggagcgcagtcaccaaaacttgtcctttcagtttagccttaaccggcgcatgacttcaagactaactcctctaaatcaattaccagtggctgctgccagtggtgcttttgcatgtctttccgggttggactcaagacgatagttaccggataaggcgcagcggtcggactgaacggggggttcgtgcatacagtccagcttggagcgaactgcctacccggaactgagtgtcaggcgtggaatgagacaaacgcggccataacagcggaatgacaccggtaaaccgaaaggcaggaacaggagagcgcacgagggagccgccagggggaaacgcctggtatctttatagtcctgtcgggtttcgccaccactgatttgagcgtcagatttcgtgatgcttgtcaggggggcggagcctatggaaaaacggctttgccgcggccctctcacttccctgttaagtatcttcctggcatcttccaggaaatctccgccccgttcgtaagccatttccgctcgccgcagtcgaacgaccgagcgtagcgagtcagtgagcgaggaagcggaatatatcctgtatcacatattctgctgacgcaccggtgcagccttttttctcctgccacatgaagcacttcactgacaccctcatcagtgccaacatagtaagccagtatacactccgctagcgctgatgtccggcggtgcttttgccgttacgcaccaccccgtcagtagctgaacaggagggacagctgatagaaacagaagccactggagcacctcaaaaacaccatcatacactaaatcagtaagttggcagcatcacccgacgcactttgcgccgaataaatacctgtgacggaagatcacttcgcagaataaataaatcctggtgtccctgttgataccgggaagccctgggccaacttttggcgaaaatgagacgttgatcggcacgtaagaggttccaactttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccactaggatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaatttttttaaggcagttattggtgcccttaaacgcctggttgctacgcctgaataagtgataataagcggatgaatggcagaaattcgaaagcaaattcgacccggtcgtcggttcagggcagggtcgttaaatagccgcttatgtctattgctggtttaccggtttattgactaccggaagcagtgtgaccgtgtgcttctcaaatgcctgaggccagtttgctcaggctctccccgtggaggtaataattgacgatatgatcatttattctgcctcccagagcctgataaaaacggttagcgcttcgttaatacagatgtaggtgttccacagggtagccagcagcatcctgcgatgcagatccggaacataatggtgcagggcgcttgtttcggcgtgggtatggtggcaggccccgtggccgggggactgttgggcgctgccggcacctgtcctacgagttgcatgataaagaagacagtcataagtgcggcgacgatagtcatgccccgcgcccaccggaaggagctaccggcagcggtgcggactgttgtaactcagaataagaaatgaggccgctcatggcgttctgttgcccgtctcactggtgaaaagaaaaacaaccctggcgccgcttctttgagcgaacgatcaaaaataagtggcgccccatcaaaaaaatattctcaacataaaaaactttgtgtaatacttgtaacgctagatctgggaacctgatcatgtagatcgaatggactctaaatccgttcagccgggttagattcccggggtttccgccaactagtatccttagcgaaagctaaggattttttttaagcttggcactggccgtcgttttacaacgtcgtgactgggaaaaccctggcgttacccaacttaatcgccttgcagcacatccccctttcgccagacgctctcccttatgcgactcctgcattaggaagcagcccagtagtaggttgaggccgttgagcaccgccgccgcaaggaatggtgcatgcaaggagcccgagatgcgccgcgtgcggctgctggagatggcggacgcgatggatatgttctgccaagggttggtttgcgcattcacagttctccgcaagaattgattggctccaattcttggagtggtgaatccgttagcgaggtgccgccggcttccattcaggtcgaggtggcccggctccatgcaccgcgacgcaacgcggggaggcagacaaggtatagggcggcgcctacaatccatgccaacccgttccatgtgctcgccgaggcggcataaatcgccgtgacgatcagcggtccagtgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatccacaggacgggtgtggtcgccatgatcgcgtagtcgatagtggctccaagtagcgaagcgagcaggactgggcggcggccaaagcggtcggacagtgctccgagaacgggtgcgcatagaaattgcatcaacgcatatagcgctagcagcacgccatagtgactggcgatgctgtcggaatggacgatatcccgcaagaggcccggcagtaccggcataaccaagcctatgcctacagcatccagggtgacggtgccgaggatgacgatgagcgcattgttagatttcatacacggtgcctgactgcgttagcaatttaactgtgataaactaccgcattaaagcttatcgatgataagctgtcaaacatgagaattacaacttatatcgtatggggctgacttcaggtgctacattgctcaaagatgcaggggtaaaagctaaccgcatctttaccgacaaggcatccggcagttcaacagatcgggaagggctggatttgctgaggatgaaggtggaggaaggtgatgtcattctggtgaagaagctcgaccgtcttggccgcgacaccgccgacatgatccaactgataaaagagtttgatgctcagggtgtagcggttcggtttattgacgacgggatcagtaccgacggtgatatggggcaaatggtggtcaccatcctgtcggctgtggcacaggctgaacgccggaggatcctccgggccgttgcttcgcaacgttcaaatccgctcccggcggatttgtcctactcaggagagcgttcaccgacaaacaacagataaaacgaaaggcccagtctttcgactgagcctttcgttttatttgatgcctggcagttccctactctcgcatggggagaccccacactaccatcggcgctacggcgtttcacttctgagttcggcatggggtcaggtgggaccaccgcgctactgccgccaggcaaattctgttttatcagaccgcttctgcgttctgatttaatctgtatcaggctgaaaatcttctctcatccgacgtcttaggcgaaggcgaagtccgactctaagatgtcacggaggttcaagttacctttagccggaagtgctggcattttgtccaattgagactcgtgcaactggtcagcgaactggtcgtagaaatcagccagtacatcacaagactcatatgtgtcaaccatagtttcgcgcactgctttgaacaggttcgcagcgtcagccggaatggtaccgaaggagtcgtgaatcagtgcaaaagattcgattccgtacttctcgtgtgcccacactacagtcttacgaaggtggctaccgtcttggctgtgtacaaagttaggagcgataccagactcctgtttgtgtgcatcaatctcgctatctttgttggtgttaatggtaggctgtaagcggaactgaccgaggaacatcaggttcaagcgcgtctgaataggcttcttgtattcctgccacacagggaaaccatcaggagttacccaatgcacagcgcaacgcttgcgaagaatctctccagtcttcttatctttgacctcagcagccagcagcttagcagcagacttaagccagttcattgcttcaaccgcagctaccaccgtcacgctcacagattcccaaatcagcttagccatgtatccagcagcctgattcggctgagtgaacatcagacccttgccggaatcaatagctggctgaatggtatcttccagcacttgttgacggaagccgaactctttggacccgtaagccagcgtcatgactgaacgcttagtcacactgcgagtaacaccgtaagccagccattgaccagccagtgccttagtgcccagcttgactttctcagagatttcaccagtgttctcatcggtcacggtaactacttcgttatcggtcccattgattgcgtctgcttgtagaatctcgttgactttcttagcaacaatcccgtagatgtcctgaacggtttcactaggaagcaagttaaccgcgcgaccacctacctcatctcggagcatcgcggagaagtgctggatgccagagcaagacccgtcaaacgccagcggaagggagcagttatagctcaggccgtggtgctgtaccccagcgtactcaaagcagaacgcaaggaagcagaacggagaatcttgctcagcccaccaagtgttctccagtggagacttagcgcaagccatgatgttctcgtggttttcctcaatgaacttgatgcgctcagggaacggaaccttatcgacacccgcacagtttgcaccgtggattttcagccagtagtaaccttccttaccgattggtttacctttcgccagcgtaagcagtcctttggtcatatcgttaccttgcgggttgaacattgacacagcgtaaacacgaccgcgccagtccatgttgtaagggaaccagatggccttatggttagcaaacttattggcttgctcaagcatgaactcaaggctgatacggcgagacttgcgagccttgtccttgcggtacacagcagcggcagcacgtttccacgcggtgagagcctcaggattcatgtcgatgtcttccggtttcatcgggagttcttcacgctcaatcgcagggatgtcctcgaccggacaatgcttccacttggtgattacgttggcgaccgctaggactttcttgttgattttccatgcggtgttttgcgcaatgttaatcgctttgtacacctcaggcatgtaaacgtcttcgtagcgcatcagtgctttcttactgtgagtacgcaccagcgccagaggacgacgaccgttagcccaatagccaccaccagtaatgccagtccacggcttaggaggaactacgcaaggttggaacatcggagagatgccagccagcgcacctgcacgggttgcgatagcctcagcgtattcaggtgcgagttcgatagtctcagagtcttgacctactacgccagcattttggcggtgtaagctaaccattccggttgactcaatgagcatctcgatgcagcgtactcctacatgaatagagtcttccttatgccacgaagaccacgcctcgccaccgagtagacccttagagagcatgtcagcctcgacaacttgcataaatgctttcttgtagacgtgccctacgcgcttgttgagttgttcctcaacgtttttcttgaagtgcttagcttcaaggtcacggatacgaccgaagcgagcctcgtcctcaatggcccgaccgattgcgcttgctacagcctgaacggttgtattgtcagcactggttaggcaagccagagtggtcttaatggtgatgtacgctacggcttccggcttgatttcttgcaggaactggaaggctgtcgggcgcttgccgcgcttagctttcacttcctcaaaccagtcgttgatgcgtgcaatcatcttagggagtagggtagtgatgagaggcttggcggcagcgttatccgcaacctcaccagctttaagttgacgctcaaacatcttgcggaagcgtgcttcacccatctcgtaagactcatgctcaagggccaactgttcgcgagctaaacgctcaccgtaatggtcagccagagtgttgaacgggatagcagccagttcgatgtcagagaagtcgttcttagcaatgttaatggtattctagtgcacggtaatcatggtcatggttaattcctcctgttagcccaaaaaaacgggtatggagaaacagtagagagttgcgataaaaagcgtcaggtaggatccgctaatcttatggataaaaatgctatggcatagcaaagtgtgacgccgtgcaaataatcaatgtggacttttctgccgtgattatagacacttttgttacgcgtttttgtcatggctttggtcccgctttgttacagaatgcttttaataagcggggttaccggtttggttagcgagaagagccagtaaaagacgcagtgacggcaatgtctgatgcaatatggacaattggtttcttctctgaatggcgggagtatgaaaagtatggctgaagcgcaaaatgatcccctgctgccgggatactcgtttaatgcccatctggtggcgggtttaacgccgattgaggccaacggttatctcgatttttttatcgaccgaccgctgggaatgaaaggttatattctcaatctcaccattcgcggtcagggggtggtgaaaaatcagggacgagaatttgtttgccgaccgggtgatattttgctgttcccgccaggagagattcatcactacggtcgtcatccggaggctcgcgaatggtatcaccagtgggtttactttcgtccgcgcgcctactggcatgaatggcttaactggccgtcaatatttgccaatacggggttctttcgcccggatgaagcgcaccagccgcatttcagcgacctgtttgggcaaatcattaacgccgggcaaggggaagggcgctattcggagctgctggcgataaatctgcttgagcaattgttactgcggcgcatggaagcgattaacgagtcgctccatccaccgatggataatcgggtacgcgaggcttgtcagtacatcagcgatcacctggcagacagcaattttgatatcgccagcgtcgcacagcatgtttgcttgtcgccgtcgcgtctgtcacatcttttccgccagcagttagggattagcgtcttaagctggcgcgaggaccaacgtatcagccaggcgaagctgcttttgagcaccacccggatgcctatcgccaccgtcggtcgcaatgttggttttgacgatcaactctatttctcgcgggtatttaaaaaatgcaccggggccagcccgagcgagttccgtgccggttgtgaagaaaaagtgaatgatgtagccgtcaagttgtcataattggtaacgaatcagacaattgacggcttgacggagtagcatagggtttgcagaatccctgcttcgtccatttgacaggcacattatgcatgccgcttcgccttcgcgcgcgaattgatctgctgcctcgcgcgtttcggtgatgacggtgaaaacctctgacacatgcagctcccggagacggtcacagcctgcagcaaaaaacccctcaagacccgtttagaggccccaaggggttatgctagttattgctcagcggtggcagcagccaactcagcttcctttcgggctttgttatttgtagagctcatccatgccatgtgtaatcccagcagcagttacaaactcaagaaggaccatgtggtcacgcttttcgttgggatctttcgaaagggcagattgtgtcgacaggtaatggttgtctggtaaaaggacagggccatcgccaattggagtattttgttgataatggtctgctagttgaacggatccatcttcaatgttgtggcgaattttgaagttagctttgattccattcttttgtttgtctgccgtgatgtatacattgtgtgagttatagttgtactcgagtttgtgtccgagaatgtttccatcttctttaaaatcaataccttttaactcgatacgattaacaagggtatcaccttcaaacttgacttcagcacgcgtcttgtagttcccgtcatctttgaaagatatagtgcgttcctgtacataaccttcgggcatggcactcttgaaaaagtcatgccgtttcatatgatccggataacgggaaaagcattgaacaccataagagaaagtagtgacaagtgttggccatggaacaggtagttttccagtagtgcaaataaatttaagggtaagttttccgtatgttgcatcaccttcaccctctccactgacagaaaatttgtgcccattaacatcaccatctaattcaacaagaattgggacaactccagtgaaaagttcttctcctttactcatatgtatatctccttcttaaagttaaacaaaattatttctagagggaaaccgttgtggtctccctatagtgagtcgtattaatttcgcgggatcg

SEQ ID NO: 101 - DNA, 4621 bp, synthetic construct
  misc_feature 1..4621: note = Plasmid
  source 1..4621: mol_type = other DNA
ctcttgcgggatggccacgatgcgtccggcgtagaggatctgctcatgtttgacagcttatcatcgatgcataatgtgcctgtcaaatggacgaagcagggattctgcaaaccctatgctactccgtcaagccgtcaattgtctgattcgttaccaattatgacaacttgacggctacatcattcactttttcttcacaaccggcacggaactcgctcgggctggccccggtgcattttttaaatacccgcgagaaatagagttgatcgtcaaaaccaacattgcgaccgacggtggcgataggcatccgggtggtgctcaaaagcagcttcgcctggtgatacgttggtcctcgcgccagcttaagacgctaatccctaactgctggcggaaaagatgtgacagacgcgacggcgacaagcaaacatgctgtgcgacgctggcgatatcaaaattgctgtctgccaggtgatcgctgatgtactgacaagcctcgcgtacccgattatccatcggtggatggagcgactcgttaatcgcttccatgcgccgcagtaacaattgctcaagcagatttatcgccagcagctccgaatagcgcccttccccttgcccggcgttaatgatttgcccaaacaggtcgctgaaatgcggctggtgcgcttcatccgggcgaaagaaccccgtattggcaaatattgacggccagttaagccattcatgccagtaggcgcgcggacgaaagtaaacccactggtgataccattcgcgagcctccggatgacgaccgtagtgatgaatctctcctggcgggaacagcaaaatatcacccggtcggcaaacaaattctcgtccctgatttttcaccaccccctgaccgcgaatggtgagattgagaatataacctttcattcccagcggtcggtcgataaaaaaatcgagataaccgttggcctcaatcggcgttaaacccgccaccagatgggcattaaacgagtatcccggcagcaggggatcattttgcgcttcagccatacttttcatactcccgccattcagagaagaaaccaattgtccatattgcatcagacattgccgtcactgcgtcttttactggctcttctcgctaaccaaaaccggtaaccccgcttattaaaagcattctgtaacaaagcgggaccaaagccatgacaaaaacgcgtaacaaaagtgtctataatcacggcagaaaagtccacattgattatttgcacggcgtcacactttgctatgccatagcatttttatccataagattagcggatcctacctgacgctttttatcgcaactctctactgtttctccatacccgtttttttgggctagaaataattttgtttaactttaagaaggagatatacatatggcataggttatcaacacgtttgacggggttgcggattatcttcagacatatcataagctacctgataattacattacaaaatcagaagcacaagccctcggctgggtggcatcaaaagggaaccttgcataggtcgctccggggaaaagcatcggcggagacatcttctcaaacagggaaggcaaactcccgggcaaaagcggacgaacatggcgtgaagcggatattaactatacatcaggcttcagaaattcagaccggattctttactcaagcgactggctgatttacaaaacaacggaccattatcagacctttacaaaaatcagataagcatgcaccattccttgcggcggcggtgctcaacggcctcaacctactactgggctgcttcctaatgcaggagtcgcataagggagagccgagatctgggaacctgatcatgtagatcgaatggactctaaatccgttcagccgggttagattcccggggtttccgccaactagtttttcaacgccatgagcggcctcatttcttattctgagttacaacagtccgcaccgctgccggtagctccttccggtgggcgcggggcatgactatcgtcgccgcacttatgactgtcttctttatcatgcaactcgtaggacaggtgccggcagcgcccaacagtcccccggccacggggcctgccaccatacccacgccgaaacaagcgccctgcaccattatgttccggatctgcatcgcaggatgctgctggctaccctgtggaacacctacatctgtattaacgaagcgctaaccgtttttatcaggctctgggaggcagaataaatgatcatatcgtcaattattacctccacggggagagcctgagcaaactggcctcaggcatttgagaagcacacggtcacactgcttccggtagtcaataaaccggtaaaccagcaatagacataagcggctatttaacgaccctgccctgaaccgacgaccgggtcgaatttgctttcgaatttctgccattcatccgcttattatcacttattcaggcgtagcaaccaggcgtttaagggcaccaataactgccttaaaaaaattacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcggcgcaaagtgcgtcgggtgatgctgccaacttactgatttagtgtatgatggtgtttttgaggtgctccagtggcttctgtttctatcagctgtccctcctgttcagctactgacggggtggtgcgtaacggcaaaagcaccgccggacatcagcgctagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttctagatttcagtgcaatttatctcttcaaatgtagcacctgaagtcagccccatacgatataagttgtaattctcatgtttgacagcattatcatcgataagctttaatgcggtagtttatcacagttaaattgctaacgcagtcaggcaccgtgtatgaaatctaacaatgcgctcatcgtcatcctcggcaccgtcaccctggatgctgtaggcataggcttggttatgccggtactgccgggc

SEQ ID NO: 102 - DNA, 5733 bp, synthetic construct
  misc_feature 1..5733: note = Plasmid
  misc_difference 5694: note = n is a, c, g, or t
  misc_difference 5717: note = n is a, c, g, or t
  misc_difference 5730: note = n is a, c, g, or t
  source 1..5733: mol_type = other DNA
gagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagctgtccctcctgttcagctactgacggggtggtgcgtaacggcaaaagcaccgccggacatcagcgctagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttctagatttcagtgcaatttatctcttcaaatgtagcacctgaagtcagccccatacgatataagttgtaattctcatgtttgacagcttatcatcgataagctttaatgcggtagtttatcacagttaaattgctaacgcagtcaggcaccgtgtatgaaatctaacaatgcgctcatcgtcatcctcggcaccgtcaccctggatgctgtaggcataggcttggttatgccggtactgccgggcctcttgcgggatatcgtccattccgacagcatcgccagtcactatggcgtgctgctagcgctatatgcgttgatgcaatttctatgcgcacccgttctcggagcactgtccgaccgctttggccgccgcccagtcctgctcgcttcgctacttggagccactatcgactacgcgatcatggcgaccacacccgtcctgtggatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcattggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatctcgggctccttgcatgcaccattccttgcggcggcggtgctcaacggcctcaacctactactgggctgcttcctaatgcaggagtcgcataagggagagcgtctggcgaaagggggatgtgctgcaaggcgattaagttgggtaacgccagggttttcccagtcacgacgttgtaaaacgacggccagtgccaagcttaaaaaaaatccttagctttcgctaaggatctgcagtggcggaaaccccgggaatctaacccggctgaacggatttagagtccattcgatctacatgatcaggttcccgcggccgcgaattcagcgttacaagtattacacaaagttttttatgttgagaatatttttttgatggggcgccacttatttttgatcgttcgctcaaagaagcggcgccagggttgtttttcttttcaccagtgagacgggcaacagaacgccatgagcggcctcatttcttattctgagttacaacagtccgcaccgctgccggtagctccttccggtgggcgcggggcatgactatcgtcgccgcacttatgactgtcttctttatcatgcaactcgtaggacaggtgccggcagcgcccaacagtcccccggccacggggcctgccaccatacccacgccgaaacaagcgccctgcaccattatgttccggatctgcatcgcaggatgctgctggctaccctgtggaacacctacggtaccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagcagatcaattcgcgcgcgaaggcgaagcggcatgcataatgtgcctgtcaaatggacgaagcagggattctgcaaaccctatgctactccgtcaagccgtcaattgtctgattcgttaccaattatgacaacttgacggctacatcattcactttttcttcacaaccggcacggaactcgctcgggctggccccggtgcattttttaaatacccgcgagaaatagagttgatcgtcaaaaccaacattgcgaccgacggtggcgataggcatccgggtggtgctcaaaagcagcttcgcctggctgatacgttggtcctcgcgccagcttaagacgctaatccctaactgctggcggaaaagatgtgacagacgcgacggcgacaagcaaacatgctgtgcgacgctggcgatatcaaaattgctgtctgccaggtgatcgctgatgtactgacaagcctcgcgtacccgattatccatcggtggatggagcgactcgttaatcgcttccatgcgccgcagtaacaattgctcaagcagatttatcgccagcagctccgaatagcgcccttccccttgcccggcgttaatgatttgcccaaacaggtcgctgaaatgcggctggtgcgcttcatccgggcgaaagaaccccgtattggcaaatattgacggccagttaagccattcatgccagtaggcgcgcggacgaaagtaaacccactggtgataccattcgcgagcctccggatgacgaccgtagtgatgaatctctcctggcgggaacagcaaaatatcacccggtcggcaaacaaattctcgtccctgatttttcaccaccccctgaccgcgaatggtgagattgagaatataacctttcattcccagcggtcggtcgataaaaaaatcgagataaccgttggcctcaatcggcgttaaacccgccaccagatgggcattaaacgagtatcccggcagcaggggatcattttgcgcttcagccatacttttcatactcccgccattcagagaagaaaccaattgtccatattgcatcagacattgccgtcactgcgtcttttactggctcttctcgctaaccaaaccggtaaccccgcttattaaaagcattctgtaacaaagcgggaccaaagccatgacaaaaacgcgtaacaaaagtgtctataatcacggcagaaaagtccacattgattatttgcacggcgtcacactttgctatgccatagcatttttatccataagattagcggatcctacctgacgctttttatcgcaactctctactgtttctccatacccgttttttgggctaacaggaggaattaaccatggttagcaaaggtgaagaactgtttaccggcgttgtgccgattctggtggaactggatggtgatgtgaatggccataaatttagcgttcgtggcgaaggcgaaggtgatgcgaccaacggtaaactgaccctgaaatttatttgcaccaccggtaaactgccggttccgtggccgaccctggtgaccaccctgacctatggcgttcagtgctttagccgctatccggatcatatgaaacgccatgatttctttaaaagcgcgatgccggaaggctatgtgcaggaacgtaccattagcttcaaagatgatggcacctataaaacccgtgcggaagttaaatttgaaggcgataccctggtgaaccgcattgaactgaaaggtattgattttaaagaagatggcaacattctgggtcataaactggaatataatttcaacagccattaggtgtatattaccgccgataaacagaaaaatggcatcaaagcgaactttaaaatccgtcacaacgtggaagatggtagcgtgcagctggcggatcattatcagcagaataccccgattggtgatggcccggtgctgctgccggataatcattatctgagcacccagagcgttctgagcaaagatccgaatgaaaaacgtgatcatatggtgctgctggaatttgttaccgccgcgggcattacccacggtatggatgaactgtataaaggcagccaccatcatcatcaccattaaagctcgagcgaagcttgngcccgaacaaaaactcatctcanaagaggatctgantag

SEQ ID NO: 103 - DNA, 35 bp, synthetic construct
  misc_feature 1..35: note = PCR primer
  source 1..35: mol_type = other DNA
aatctttataactatatgcgcaaactggaccgtgc

SEQ ID NO: 104 - DNA, 34 bp, synthetic construct
  misc_feature 1..34: note = PCR primer
  source 1..34: mol_type = other DNA
atagttataaagatttggtgctagcatagggcgc

SEQ ID NO: 105 - DNA, 30 bp, synthetic construct
  misc_feature 1..30: note = PCR primer
  source 1..30: mol_type = other DNA
atggtgtatggcgacaccctggatgtcatg

SEQ ID NO: 106 - DNA, 31 bp, synthetic construct
  misc_feature 1..31: note = PCR primer
  source 1..31: mol_type = other DNA
tgtcgccatacaccatacagctgtcgcccac

SEQ ID NO: 107 - DNA, 1365 bp, synthetic construct
  misc_feature 1..1365: note = PylRS AF A1 Mutant
  source 1..1365: mol_type = other DNA
atggataaaaaaccgctgaataccctgatctctgctactggtctgtggatgagtcgtaccggaaccattcataaaatcaaacaccacgaggttagccgttcgaaaatctatattgagatggcgtgtggcgatcatctggttgtgaacaatagccgctcttctcgtacagcacgtgcactgcgtcaccacaaatatcgtaaaacctgtaaacgttgccgtgtgtccgatgaggatctgaacaaattcctgacaaaagccaatgaggaccaaacaagcgtgaaagtgaaagtcgttagcgctcctacccgtactaaaaaagcaatgccgaaatccgttgctcgtgcccctaaaccactggaaaacactgaagcagcacaggcacagccgtctggaagcaaattctctccggccattcctgtttctacccaggagtccgtttctgttccagcaagtgtgagcaccagcattagcagtattagcaccggtgccaccgctagcgccctggttaaaggcaataccaatccgattacaagcatgtctgccccggttcaagcatcagctccagcactgacaaaatcccaaaccgatcgtctggaggttctgctgaatccgaaagacgaaatcagcctgaattccggcaaaccgtttcgtgaactggagagcgaactgctgtcacgtcgtaaaaaagacctgcaacaaatctatgccgaagaacgtgagaactatctggggaaactggaacgtgaaatcacccgctttttcgtggatcgtggctttctggagatcaaatccccgattctgattcctctggagtatatcgagcgtatgggcatcgacaatgataccgaactgagcaaacagatcttccgtgtggataaaaacttctgtctgcgccctatgctagcaccaaatcttgctaactatatgcgcaaactggaccgtgccctgcctgatcctatcaaaatcttcgagatcggcccgtgttatcgtaaagagtccgacggtaaagaacatctggaggagtttaccatgctgaactttggtcaaatgggttcaggttgtactcgtgagaacctggaaagcatcatcaccgattttctgaaccacctgggcattgacttcaaaattgtgggcgacagctgtatggtgtttggcgacaccctggatgtcatgcacggcgacctggaactgtctagtgccgttgttggaccaaggccgctggaccgtgagtggggtatcgacaaaccgtggatcggagcaggattcggtctggaacgcctgctgaaagtgaaacacgacttcaaaaacatcaaacgtgccgcccgttctgaatcgtattataacgggatctctacgaacctgtaa

SEQ ID NO: 108 - protein, 454 aa, synthetic construct
  REGION 1..454: note = Synthetic Construct
MDKKPLNTLISATGLWMSRTGTIHKIKHHEVSRSKIYIEMACGDHLVVNNSRSSRTARALRHHKYRKTCKRCRVSDEDLNKFLTKANEDQTSVKVKVVSAPTRTKKAMPKSVARAPKPLENTEAAQAQPSGSKFSPAIPVSTQESVSVPASVSTSISSISTGATASALVKGNTNPITSMSAPVQASAPALTKSQTDRLEVLLNPKDEISLNSGKPFRELESELLSRRKKDLQQIYAEERENYLGKLEREITRFFVDRGFLEIKSPILIPLEYIERMGIDNDTELSKQIFRVDKNFCLRPMLAPNLANYMRKLDRALPDPIKIFEIGPCYRKESDGKEHLEEFTMLNFGQMGSGCTRENLESIITDFLNHLGIDFKIVGDSCMVFGDTLDVMHGDLELSSAVVGPRPLDREWGIDKPWIGAGFGLERLLKVKHDFKNIKRAARSESYYNGISTNL

SEQ ID NO: 109 - DNA, 1416 bp, synthetic construct
  misc_feature 1..1416: note = PylRS AF A1 NES Mutant
  source 1..1416: mol_type = other DNA
atggcgtgcccggtgccgctgcagctgccgccgctggaacgcctgaccctggatgataaaaaaccgctgaataccctgatctctgctactggtctgtggatgagtcgtaccggaaccattcataaaatcaaacaccacgaggttagccgttcgaaaatctatattgagatggcgtgtggcgatcatctggttgtgaacaatagccgctcttctcgtacagcacgtgcactgcgtcaccacaaatatcgtaaaacctgtaaacgttgccgtgtgtccgatgaggatctgaacaaattcctgacaaaagccaatgaggaccaaacaagcgtgaaagtgaaagtcgttagcgctcctacccgtactaaaaaagcaatgccgaaatccgttgctcgtgcccctaaaccactggaaaacactgaagcagcacaggcacagccgtctggaagcaaattctctccggccattcctgtttctacccaggagtccgtttctgttccagcaagtgtgagcaccagcattagcagtattagcaccggtgccaccgctagcgccctggttaaaggcaataccaatccgattacaagcatgtctgccccggttcaagcatcagctccagcactgacaaaatcccaaaccgatcgtctggaggttctgctgaatccgaaagacgaaatcagcctgaattccggcaaaccgtttcgtgaactggagagcgaactgctgtcacgtcgtaaaaaagacctgcaacaaatctatgccgaagaacgtgagaactatctggggaaactggaacgtgaaatcacccgctttttcgtggatcgtggctttctggagatcaaatccccgattctgattcctctggagtatatcgagcgtatgggcatcgacaatgataccgaactgagcaaacagatcttccgtgtggataaaaacttctgtctgcgccctatgctagcaccaaatcttgctaactatatgcgcaaactggaccgtgccctgcctgatcctatcaaaatcttcgagatcggcccgtgttatcgtaaagagtccgacggtaaagaacatctggaggagtttaccatgctgaactttggtcaaatgggttcaggttgtactcgtgagaacctggaaagcatcatcaccgattttctgaaccacctgggcattgacttcaaaattgtgggcgacagctgtatggtgtttggcgacaccctggatgtcatgcacggcgacctggaactgtctagtgccgttgttggaccaaggccgctggaccgtgagtggggtatcgacaaaccgtggatcggagcaggattcggtctggaacgcctgctgaaagtgaaacacgacttcaaaaacatcaaacgtgccgcccgttctgaatcgtattataacgggatctctacgaacctgtaa

SEQ ID NO: 110 - protein, 471 aa, synthetic construct
  REGION 1..471: note = Synthetic Construct
MACPVPLQLPPLERLTLDDKKPLNTLISATGLWMSRTGTIHKIKHHEVSRSKIYIEMACGDHLVVNNSRSSRTARALRHHKYRKTCKRCRVSDEDLNKFLTKANEDQTSVKVKVVSAPTRTKKAMPKSVARAPKPLENTEAAQAQPSGSKFSPAIPVSTQESVSVPASVSTSISSISTGATASALVKGNTNPITSMSAPVQASAPALTKSQTDRLEVLLNPKDEISLNSGKPFRELESELLSRRKKDLQQIYAEERENYLGKLEREITRFFVDRGFLEIKSPILIPLEYIERMGIDNDTELSKQIFRVDKNFCLRPMLAPNLANYMRKLDRALPDPIKIFEIGPCYRKESDGKEHLEEFTMLNFGQMGSGCTRENLESIITDFLNHLGIDFKIVGDSCMVFGDTLDVMHGDLELSSAVVGPRPLDREWGIDKPWIGAGFGLERLLKVKHDFKNIKRAARSESYYNGISTNL

SEQ ID NO: 111-112 - skipped (2 entries)

SEQ ID NO: 113 - DNA, 72 bp, synthetic construct
  misc_feature 1..72: note = pyrrolysyl tRNA codimg sequence
  source 1..72: mol_type = other DNA
ggaaacctgatcatgtagatcgaatggactctaaatccgttcagccgggttagattcccggggtttccgcca

(19) United States
(12) Patent Application Publication
(10) Pub. No.: US 2025/0327057 A1
(43) Pub. Date: Oct. 23, 2025

(54) NOVEL AMINOACYL-TRNA SYNTHETASE VARIANTS FOR GENETIC CODE EXPANSION IN EUKARYOTES

(71) Applicant: VERAXA Biotech GmbH, Heidelberg (DE)

(72) Inventor: Christine KÖHLER, Forst (DE)

(21) Appl. No.: 18/689,286

(22) PCT Filed: Sep. 5, 2022
(86) PCT No.: PCT/EP2022/074542

§ 371 Date: Mar. 5, 2024

(30) Foreign Application Priority Data
Sep. 6, 2021 (EP) 21195008.4

(51) Int. Cl. C12N 9/00; C12P 21/00

(52) U.S. Cl. CPC C12N 9/93; C12P 21/00; C12Y 601/01026

(57) ABSTRACT

The present invention relates to novel, improved aminoacyl-tRNA synthetase variants, which are useful for genetic code expansion. The present invention also relates to corresponding coding sequences and eukaryotic cell lines comprising such coding sequences. The present invention also relates to methods of preparing a protein of interest (POI) comprising one or more unnatural amino acid residues incorporated by means of the novel aminoacyl-tRNA synthetase variants. The present invention also relates to a method of preparing polypeptide conjugates, wherein a POI generated by means of the present aminoacyl-tRNA synthetase variants is reacted with one or more conjugation partner molecules. Finally, the present invention relates to a kit comprising the required constituents for preparing such POIs comprising one or more unnatural amino acid residues.

### CROSS-REFERENCE TO RELATED APPLICATION

This application is a 35 U.S.C § 371 application of International Application No. PCT/EP2022/074542, filed on Sep. 5, 2022, which claims priority to European Patent Application No. 21195008.4 that was filed on Sep. 6, 2021. The entire content of the applications referenced above are hereby incorporated by reference herein.

### SEQUENCE LISTING

The instant application contains a Sequence Listing which has been submitted electronically in XML file format and is hereby incorporated by reference in its entirety. Said XML copy, created on Oct. 8, 2024, is named 05710_066US1_SL.xml and is 220,841 bytes in size.

### FIELD OF THE INVENTION

The present invention relates to novel, improved aminoacyl-tRNA synthetase variants, which are useful for genetic code expansion. The present invention also relates to corresponding coding sequences and eukaryotic cell lines comprising such coding sequences. The present invention also relates to methods of preparing a protein of interest (POI) comprising one or more unnatural amino acid residues incorporated by means of the novel aminoacyl-tRNA synthetase variants. The present invention also relates to a method of preparing polypeptide conjugates, wherein a POI generated by means of the present aminoacyl-tRNA synthetase variants is reacted with one or more conjugation partner molecules. Finally, the present invention relates to a kit comprising the required constituents for preparing such POIs comprising one or more unnatural amino acid residues.

### BACKGROUND OF THE INVENTION

Genetic code expansion (GCE) is a versatile tool for the site-specific incorporation of non-canonical amino acids (ncAAs) into proteins. These ncAA can be used for protein engineering like fluorescent labeling of proteins or conjugation of toxic payloads with antibodies. For the incorporation of the ncAA into the protein during translation, a special aminoacyl-tRNA synthetase/tRNA (aaRS/tRNA) pair is necessary, which can recognize the ncAA and otherwise is orthogonal to the host organism.

There are several such aaRS/tRNA pairs known nowadays, such as the PylRS/tRNA^{Pyl} from archaea organisms, like Methanosarcina mazei, Methanosarcina barkeri or Methanomethylophilus alvus. The PylRS synthetase contains a binding site, which originally recognizes pyrrolysine. By chaining specific amino acids in this binding site, a variety of ncAAs can be recognized by this synthetase.

There are over 200 ncAAs meanwhile existing which can be used in combination with the PylRS/tRNA^{Pyl} pair.

A particular group of ncAAs comprises H-Lys(Boc)-OH (“Boc”), Cyclooctyne-Lysine (“SCO”), Bicyclo [6.1.0] nonyne-Lysine (“BCN”), trans-Cyclooct-2-en-Lysine (“TCO*A”) and trans-Cyclooct-4-en-Lysine (“TCO-E”), for example described in Reinkemeier et al., Eur J Chem (2021) 27 (19) 6094-6099. These ncAAs reacting via click chemistry with tetrazines are of high interest, because this reaction is very fast and bioorthogonal.

Two amino acids in the binding pocket of PylRS are well known to be important to facilitate the incorporation of such bulky amino acids. With reference to PylRS from Methanosarcina mazei, the following sequence positions were modified: Tyrosine 306 has to be changed to alanine (Y306A) and in addition, tyrosine 384 has to be mutated to phenylalanine (Y384F) to be able to incorporate bulky amino acids resulting in the PylRS variant PylRS^{AF}. The efficiency of incorporation of the different ncAAs can vary from very low to very high. Especially the ncAA, TCO-E is not accepted very well by PylRS^{AF}.

European Patent Application EP-A-2 192 185 discloses mutant pyrrolysyl-tRNA synthetases derived from M. mazei. In particular, single mutants Y306A and Y384F, as well as corresponding double mutants in position 306 and 384 are described therein. Moreover, double mutants L309A and C348A are described therein. Said mutants are reported to be capable of aminoacylating Boc.

European Patent Application EP-A-2 221 370 describes a method of producing unnatural proteins by applying a modified aminoacyl-tRNA synthetase derived from M. mazei, comprising at least one of the following substitutions: A302F, Y306A, L309A, N346S, C348V/I and Y384F.

European Patent Application EP-A-2 804 872 describes a method for incorporating an amino acid comprising a BCN group into a polypeptide using an orthogonal codon encoding it and an orthogonal PylRS synthetase derived from M. mazei comprising mutations selected from L301V. L305I, Y306F, L309A and C348F.

The problem to be solved by the present invention thus relates to the provision of novel aminoacyl tRNA synthetases which show an improved incorporation of bulky ncAAs into the amino acid sequence of a POI, in particular of ncAA residues selected from trans-Cyclooct-2-en-Lysine (“TCO*A”), trans-Cyclooct-4-en-Lysine (“TCO-E”) and H-Lys(Boc)-OH (“Boc”) and combinations thereof, and in particular an improved incorporation thereof in comparison to the prior art mutant PylRS^{AF}.

### SUMMARY OF THE INVENTION

The above mentioned problem could, surprisingly, be solved by the systematic mutation of certain key positions of the prior art synthetase PylRS^{AF} and performing subsequent back mutations in certain positions of the obtained mutants.

The present inventors performed a library selection using PylRS^{AF} from Methanosarcina mazei as the parental gene with five positions mutated to any of the 20 possible amino acids. This gene library was selected in a repetitive positive and negative screening. Out of this screening the inventors were able to obtain several variants of the PylRS^{AF} synthetase with two or three additional amino acid residues mutated which we call PylRS AF A1, PylRS AF B11, PylRS AF C11, PylRS AF G3 and PylRS AF H12. To test, if the mutations Y306A and Y384F are really important for the incorporation of bulky ncAAs, these amino acids were changed back to their original amino acids by site directed mutagenesis in the case of the PylRS AF A1 variant. Therefore, the new variant does not contain the Y306A and Y384F mutations and is called PylRS A1. A further mutant which does not contain the Y306A and Y384F mutations was prepared which is called PylRS MMA has the mutations 306M 309M 348A (Table 1).

TABLE 1
Overview PylRS variants

| Synthetase | SEQ ID | | | | | |
|---|---|---|---|---|---|---|
| name | NO | 306 | 309 | 348 | 384 | 405 |
| PylRS WT | 56 | Y | L | C | Y | I |
| PylRS AF | 68 | A | L | C | F | I |
| PylRS AF A1 | 108 | A | M | G | F | R |
| PylRS AF B11 | | A | V | G | F | N |
| PylRS AF C11 | | A | C | G | F | I |
| PylRS AF G3 | | A | V | A | F | R |
| PylRS AF H12 | | A | V | G | F | R |
| PylRS A1 | 70 | Y | M | G | Y | R |
| PylRS MMA | 72 | M | M | A | Y | I |

These new PylRS variants were tested in HEK293T cells utilizing a fluorescent reporter by fluorescent flow cytometry (FFC). The reporter contains an infrared fluorescent protein (iRFP) fused to green fluorescent protein (GFP) which harbors an amber stop codon at position Y39 and is called iRFP-GFP^{Y39TAG}. All of the new variants are able to incorporate both ncAAs tested here. In addition all the new variants show a higher green signal in comparison to the PylRS^{AF} when TCO-E is used. Even more surprisingly it was observed, that the mutations Y306A and Y384F PylRS^{AF} are not really important for the incorporation of bulky ncAAs. In particular, the new “non-AF” variant PylRS A1 contains mutations which makes it the better synthetase for bulky ncAAs compared to the already known PylRS AF variant.

In analogy to PylRS A1 further “non-AF” mutants, namely PylRS B11, PylRS C11, PylRS G3 and PylRS H12 were provided (see Table 2).

TABLE 2
Overview non-AF PylRS variants

| Synthetized | SEQ ID | | | | | |
|---|---|---|---|---|---|---|
| name | NO: | 306 | 309 | 348 | 384 | 405 |
| PylRS WT | 56 | Y | L | C | Y | I |
| PylRS AF | 68 | A | L | C | F | I |
| PylRS A1 | 70 | Y | M | G | Y | R |
| PylRS B11 | 82 | Y | V | G | Y | N |
| PylRS C11 | 84 | Y | C | G | Y | I |
| PylRS G3 | 86 | Y | V | A | Y | R |
| PylRS H12 | 88 | Y | V | G | Y | R |

### DESCRIPTION OF DRAWINGS

FIG. 1: Plasmid maps for the following plasmids as used according to the present invention are shown:

Reporter plasmid pCI-iRFP-EGFP^{Y39TAG}-6His (SEQ ID NO: 97), (FIG. 1A, top). Plasmid pCMV-NES-PylRS^{AF}-U6tRNArv (SEQ ID NO: 98) (FIG. 1A, bottom).

Plasmid, pBK-PylRS^{WT} (SEQ ID NO: 99) (FIG. 1B, top). Plasmid pREP-PylT (SEQ ID NO: 100) (FIG. 1B, bottom).

Plasmids pYOBB2-PylT (SEQ ID NO: 101) (FIG. 1C, top) and pALS-sfGFP^{N150TAG}-MbPyl-tRNA (SEQ ID NO: 102) (FIG. 1C, bottom).

FIG. 2: Data of FFC experiments of different PylRS variants (PylRS AF, PylRS AF A1, PylRS AF B11, PylRS AF C11, PylRS AF G3 and PylRS AF H12) with different ncAAs are shown. In particular, their ability to incorporate TCO*A (FIG. 2A) as well as TCO-E (FIG. 2B) is shown. FIG. 2C shows the expression profile in the absence of the ncAA.

FIG. 3: This figure illustrates in bar plots the data obtained by FFC analysis of different PylRS variants PylRS AF, PylRS AF A1, PylRS A1 and PylRS MMA. Their ability to incorporate TCO-E (FIG. 3A), TCO*A (FIG. 3B) as well as Boc (FIG. 3C) is shown. Each bar plot on top shows the ratio between the mean GFP signal obtained by FFC divided by the mean iRFP signal, which reflects the incorporation efficiency. Each middle bar plot shows the same data but normalized to the ratio observed for the PylRS AF variant at 100 μM ncAA used. Each bar plot on the bottom shows the ratio of mean GFP/mean iRFP normalized to the ratio mean GFP/mean iRFP obtained for the PylRS AF variant at the desired ncAA concentration. These bar plot illustrates on how much higher the incorporation efficiency is for each variant compared to PylRS AF.

FIG. 4: A sequence alignment of different archaeal PylRS proteins is shown.

FIG. 5: FFC data for evaluating the incorporation efficiency of variant PylRS A1 at different concentrations of the bulky ncAA cyclooctyne-lysine (SCO) are shown.

### DETAILED DESCRIPTION OF THE INVENTION

### A. Abbreviations

Bps=base pairs
BCN=2-amino-6-(9-biocyclo[6.1.0]non-4-ynylmethoxycarbonylamino)hexanoid acid
BOC=2-amino-6-(tert-butoxycarbonylamino)hexanoic acid, in the examples “BOC” specifically designates (2S)-2-amino-6-(tert-butoxycarbonylamino)hexanoic acid=Boc-L-Lys-OH═N-α-tert-butyloxycarbonyl-L-lysine
Cm=Chloramphenicol
CMV=cytomegalovirus
Crm1=chromosomal region maintenance 1, also known as karyopherin exportin 1
DH10B=E. coli strain F-mcrA Δ(mrr-hsdRMS-mcrBC) φ80lacZΔM15 ΔlacX74 recA1 endA1 araD139 Δ (ara-leu)7697 galU galK λ-rpsL (Str^{R}) nupG
dsRNA=double-stranded RNA
dSTORM=direct stochastic optical reconstruction microscopy
E. coli BL21(DE3)AI=E. coli strain B F⁻ ompT gal dcm lon hsdS_{B}(r_{B}⁻m_{B}⁻)λ(DE3 [lacI lacUV5-T7p07 ind1 sam7 nin5]) [malB⁺]_{K-12}(λ^{S}) araB::T7RNAP-tetA
FBS=fetal bovine serum
GCE=genetic code expansion
GFP=green fluorescent protein
sfGFP=superfolder green fluorescent protein
IPTG=isopropyl β-D-1-thiogalactopyranoside
iRFP=infrared fluorescent protein
Kan=Kanamycin
ncAA=non-canonical amino acid
NES=nuclear export signal
NLS=nuclear localization signal
NNK=particular type of degenerate code used for saturation mutagenesis
O-tRNA=orthogonal tRNA
O-RS=orthogonal RS
PAINT=point accumulation for imaging in nanoscale topography
PBS=phosphate buffered saline
POI=polypeptide of interest,
pRS=prokaryotic RS
ptRNA=prokaryotic tRNA
ptRNA-ribozyme=prokaryotic RNA molecule comprising a ptRNA and at least one ribozyme
PylRS=pyrrolysyl tRNA synthetase
PylRS^{AF}=mutant M. mazei pyrrolysyl tRNA synthetase comprising amino acid substitutions Y306A and Y384F
RP-HPLC=reversed phase high-performance liquid chromatography
RS=aminoacyl tRNA synthetase
RT=room temperature
SCO=2-amino-6-(cyclooct-2-yn-1-yloxycarbonylamino) hexanoic acid

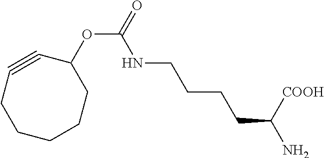 [SMILES: N[C@@H](CCCCNC(=O)OC1C#CCCCCC1)C(=O)O]

SOC=transformation medium (for example: 0.5% Yeast Extract; 2% Tryptone; 10 mM NaCl, 2.5 mM KCl; 10 mM MgCl₂; 10 mM MgSO₄; 20 mM Glucose)
SPAAC=(copper-free) strain promoted alkyne-azide cycloaddition
SPIEDAC=(copper-free) strain promoted inverse-electron-demand Diels-Alder cycloaddition
SRM=super-resolution microscopy
SV40=simian vacuolating virus 40
T7RNAP=T7 RNA polymerase
TCO-E=trans-cyclooct-4-en-L-lysine

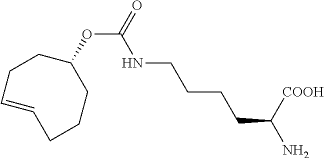

TCO*A=trans-cyclooct-2-en-L-lysine

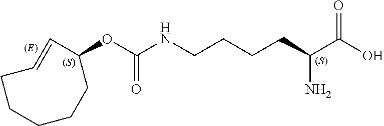

Tet=Tetracycline
tetR=tetracycline repressor
tetO=tetracycline operator
tRNA^{Pyl}=tRNA that can be acylated with pyrrolysine by a wild-type or modified PylRS and has an anticodon that, for site-specific incorporation of the ncAA into a POI, is preferably the reverse complement of a selector codon. (In the tRNA^{Pyl} used in the examples, the anticodon is CUA.)
UNAA=unnatural amino acid, synonym to ncAA
U6 promoter=promoter that normally controls expression of the U6 RNA (a small nuclear RNA) in mammalian cells

### B. Definitions

Unless otherwise defined herein, scientific and technical terms used in connection with the present invention shall have the meanings that are commonly understood by those of ordinary skill in the art. The meaning and scope of the terms should be clear, however, in the event of any latent ambiguity, definitions provided herein take precedent over any dictionary or extrinsic definition. Further, unless otherwise required by context, singular terms shall include pluralities and plural terms shall include the singular.

Pyrrolysyl tRNA synthetase (PylRS) is an aminoacyl tRNA synthetase (RS). RSs are enzymes capable of acylating a tRNA with an amino acid or amino acid analog. Expediently, the PylRS of the invention is enzymatically active, i.e. is capable of acylating a tRNA (tRNA^{Pyl}) with a certain amino acid or amino acid analog, preferably with an UNAA or salt thereof

The term “archaeal pyrrolysyl tRNA synthetase” (abbreviated as “archaeal PylRS”) as used herein refers to a PylRS, wherein at least a segment of the PylRS amino acid sequence, or the entire PylRS amino acid sequence, has at least 60%, at least 70%, at least 80%, at least 90%, at least 95%, at last 99%, or 100% sequence identity to the amino acid sequence of a naturally occurring PylRS from an archaeon, or to the amino acid sequence of an enzymatically active fragment of such naturally occurring PylRS.

The PylRS of the present invention may comprise a mutant archaeal PylRS, or an enzymatically active fragment thereof.

Generally, “mutant archaeal PylRSs” or “mutated archaeal PylRSs” differ from the corresponding wildtype PylRSs in comprising additions, substitutions and/or deletions of one or more than one amino acid residue. Preferably, these are modifications which improve PylRS stability, alter PylRS substrate specificity and/or enhance PylRS enzymatic activity. Particularly preferred “mutant archaeal PylRSs” or “mutated archaeal PylRSs” are described in more detail herein below.

The term “nuclear export signal” (abbreviated as “NES”) refers to an amino acid sequence which can direct a polypeptide containing it (such as a NES-containing PylRS of the invention) to be exported from the nucleus of a eukaryotic cell. Said export is believed to be mostly mediated by Crm1 (chromosomal region maintenance 1, also known as karyopherin exportin 1). NESs are known in the art. For example, the database ValidNESs (http://validness.ym.edu.tw/) provides sequence information of experimentally validated NES-containing proteins. Further. NES databases like, e.g., NESbase 1.0 (www.cbs.dtu.dk/databased/NESbase-1.0/; see Le Cour et al., Nucl Acids Res 31(1), 2003) as well as tools for NES prediction like NetNES (www.cbs.dtu.dk/services/NetNES/; see La Cour et al., La Cour et al., Protein Eng Des Sel 17(6):527-536, 2004), NESpredictor (NetNES, http://www.cbs.dtu.dk/; see Fu et al., Nucl Acids Res 41:D338-D343, 2013; La Cour et al., Protein Eng Des Sel 17(6):527-536, 2004)) and NESsential (a web interface combined with ValidNESs) are available to the public. Hydrophobic leucine-rich NESs are most common and represent the best characterized group of NESs to date. A hydrophobic leucine-rich NES is a non-conservative motif having 3 or 4 hydrophobic residues. Many of these NESs comprise the conserved amino acid sequence pattern LxxLxL (SEQ ID NO:111) or LxxxLXL (SEQ ID NO:112), wherein each L is independently selected from leucine, isoleucine, valine, phenylalanine and methionine amino acid residues, and each x is independently selected from any amino acid (see La Cour et al., Protein Eng Des Sel 17(6):527-536, 2004).

The term “nuclear localization signal” (abbreviated as “NLS”, also referred to in the art as “nuclear localization sequence”) refers to an amino acid sequence which can direct a polypeptide containing it (e.g., a wild-type archaeal PylRS) to be imported into the nucleus of a eukaryotic cell. Said export is believed to be mediated by binding of the NLS-containing polypeptide to importin (also known as karyopherin) so as to form a complex that moves through a nuclear pore. NLSs are known in the art. A multitude of NLS databases and tools for NLS prediction are available to the public, such as NLSdb (see Nair et al., Nucl Acids Res 31(1), 2003), cNLS Mapper (www.nls-mapper.aib.keio.ac.jp: see Kosugi et al., Proc Natl Acad Sci USA. 106(25):10171-10176, 2009; Kosugi et al., J Biol Chem 284(1):478-485, 2009). SeqNLS (see Lin et al., PLOS One 8(10):e76864, 2013), and NucPred (www.sbc.su.se/˜maccallr/nucpred/; see Branmeier et al., Bioinformatics 23(9):1159-60, 2007).

Mutant archaeal PylRSs of the invention as defined above can be further modified by removing the NLS optionally present in said naturally occurring PylRS where the mutant is derived from and/or by introducing at least one NES. The NLS in the naturally occurring PylRS can be identified using known NLS detection tools such as, e.g., cNLS Mapper.

The removal of a NLS from and/or the introduction of a NES into an archaeal PylRS or mutant thereof, can change the localization of the thus modified polypeptide when expressed in a eukaryotic cell, and in particular can avoid or reduce accumulation of the polypeptide in the nucleus of the eukaryotic cell. Thus, the localization of a PylRS mutant of the invention expressed in a eukaryotic cell can be changed compared to a PylRS or PylRS mutant, which differs from the PylRS mutant of the invention in that it (still) comprises the NLS and lacks the NES.

Where the archaeal PylRS of the invention comprises a NES but (still) comprises an NLS, the NES is preferably chosen such that the strength of the NES overrides the NLS preventing an accumulation of the PylRS in the nucleus of a eukaryotic cell.

Removal of the NLS from a wild-type or mutant PylRS and/or introduction of a NES into the wild-type or mutant PylRS so as to obtain a PylRS of the invention do not abrogate PylRS enzymatic activity. Preferably. PylRS enzymatic activity is maintained at basically the same level. i.e. the PylRS of the invention has at least 50%, at least 60%, at least 70%, at least 80%, at least 90%, or at least 91, 92, 93, 94, 95, 96, 97, 98 or 99% of the enzymatic activity of the corresponding wild-type or mutant PylRS.

The NES is expediently located within the PylRS or mutant PylRS of the invention such that the NES is functional. For example, a NES can be attached to the C-terminus (e.g., C-terminal of the last amino acid residue) or the N-terminus (e.g., in between amino acid residue 1, the N-terminal methionine, and amino acid residue 2) of a wild-type or mutant archaeal PylRS.

The disclosure of WO2018/06948 disclosing mutated PylRSs modified by the incorporation of NES and/deletion of NLS sequences is herewith explicitly referred to and incorporated by reference.

The PylRS mutants of the invention are used in tRNA^{Pyl}/PylRS(mutant) pairs, wherein the PylRS mutant is capable of acylating the tRNA^{Pyl}, preferably with an UNAA or a salt thereof.

Unless indicated otherwise. “tRNA^{Pyl}”, as used herein, refers to a tRNA that can be acylated (essentially selectively and in particular selectively) by a PylRS mutant of the invention. The tRNA^{Pyl} described herein in the context of the present invention may be a wildtype tRNA that can be acylated by a PylRS with pyrrolysine, or a mutant of such tRNA, e.g., a wildtype or a mutant tRNA from an archaeon, for example from a Methanosarcina species. e.g. M. mazei or M. barkeri. For site-specific incorporation of the UNAA, into a POI, the anticodon comprised by the tRNA^{Pyl} used together with the PylRS of the invention is expediently the reverse complement of a selector codon. In particular embodiments, the anticodon of the tRNAPMI is the reverse complement of the amber stop codon. For other applications such as, e.g., proteome labeling (Elliott et al., Nat Biotechnol 32(5):465-472, 2014), the anticodon comprised by the tRNA^{Pyl} used together with the PylRS of the invention may be a codon recognized by endogenous tRNAs of the eukaryotic cells.

The term “selector codon” as used herein refers to a codon that is recognized (i.e. bound) by the tRNA^{Pyl} in the translation process and is not recognized by endogenous tRNAs of the eukaryotic cell. The term is also used for the corresponding codons in polypeptide-encoding sequences of polynucleotides, which are not messenger RNAs (mRNAs). e.g. DNA plasmids. Preferably, the selector codon is a codon of low abundance in naturally occurring eukaryotic cells. The anticodon of the tRNA^{Pyl} binds to a selector codon within an mRNA and thus incorporates the UNAA site-specifically into the growing chain of the polypeptide encoded by said mRNA. The known 64 genetic (triplet) codons code for 20 amino acids and three stop codons. Because only one stop codon is needed for translational termination, the other two can in principle be used to encode non-proteinogenic amino acids. For example, the amber codon, UAG, has been successfully used as a selector codon in in vitro and in vivo translation systems to direct the incorporation of unnatural amino acids. Selector codons utilized in methods of the present invention expand the genetic codon framework of the protein biosynthetic machinery of the translation system used. Specifically, selector codons include, but are not limited to, nonsense codons, such as stop codons, e.g., amber (UAG), ochre (UAA), and opal (UGA) codons; codons consisting of more than three bases (e.g., four base codons); and codons derived from natural or unnatural base pairs. For a given system, a selector codon can also include one of the natural three base codons (i.e. natural triplets), wherein the endogenous translation system does not (or only scarcely) use said natural triplet, e.g., a system that is lacking a tRNA that recognizes the natural triplet or a system wherein the natural triplet is a rare codon.

A recombinant tRNA that alters the reading of an mRNA in a given translation system (e.g. an eukaryotic cell) such that it allows for reading through, e.g., a stop codon, a four base codon, or a rare codon, is termed “suppressor tRNA”. The suppression efficiency for a stop codon serving as a selector codon (e.g., the amber codon) depends upon the competition between the (aminoacylated) tRNA^{Pyl} (which acts as suppressor tRNA) and the release factor (e.g. RF1) which binds to the stop codon and initiates release of the growing polypeptide chain from the ribosome. Suppression efficiency of such stop codon can therefore be increased using a release factor-(e.g. RF1−) deficient strain.

A polynucleotide sequence encoding a “polypeptide of interest” or “POI” can comprise one or more, e.g., two or more, more than three, etc., codons (e.g. selector codons) which are the reverse complement of the anticodon comprised by the tRNA^{Pyl}. Conventional site-directed mutagenesis can be used to introduce said codon(s) at the site of interest into a polynucleotide sequence, to generate a POI-encoding polynucleotide sequence.

The PylRS mutants and tRNA^{Pyl} of the present invention are preferably orthogonal.

The term “orthogonal” as used herein refers to a molecule (e.g., an orthogonal tRNA and/or an orthogonal RS) that is used with reduced efficiency by a translation system of interest (e.g., a eukaryotic cell used for expression of a POI as described herein). “Orthogonal” refers to the inability or reduced efficiency, e.g., less than 20% efficient, less than 10% efficient, less than 5% efficient, or e.g., less than 1% efficient, of an orthogonal tRNA or an orthogonal RS to function with the endogenous RSs or endogenous tRNAs, respectively, of the translation system of interest.

Accordingly, in particular embodiments of the invention, any endogenous RS of the eukaryotic cell of the invention catalyzes acylation of the (orthogonal) tRNA^{Pyl} with reduced or even zero efficiency, when compared to acylation of an endogenous tRNA by the endogenous RS, for example less than 20% as efficient, less than 10% as efficient, less than 5% as efficient or less than 1% as efficient. Alternatively or additionally, the (orthogonal) PylRS of the invention acylates any endogenous tRNA of the eukaryotic cell of the invention with reduced or even zero efficiency, as compared to acylation of the tRNA^{Pyl} by an endogenous RS of the cell, for example less than 20% as efficient, less than 10% as efficient, less than 5% as efficient or less than 1% as efficient.

Unless indicated differently, the terms “endogenous tRNA” and “endogenous aminoacyl tRNA synthetase” (“endogenous RS”) used therein refer to a tRNA and an RS, respectively, that was present in the cell ultimately used as translation system prior to introducing the PylRS of the invention and the tRNA^{Pyl}, respectively, used in the context of the present invention.

The term “translation system” generally refers to a set of components necessary to incorporate a naturally occurring amino acid in a growing polypeptide chain (protein). Components of a translation system can include, e.g., ribosomes, tRNAs, aminoacyl tRNA synthetases (RS), mRNA and the like. Translation systems include artificial mixture of said components, cell extracts and living cells, e.g. living eukaryotic cells.

The pair of PylRS and tRNA^{Pyl} used for preparing a POI according to the present invention is preferably orthogonal in that the tRNA^{Pyl}, in the eukaryotic cell used for preparing the POI, is preferentially acylated by the PylRS of the invention with an UNAA or a salt thereof (UNAA). Expediently, the orthogonal pair functions in said eukaryotic cell such that the cell uses the UNAA-acylated tRNA^{Pyl} to incorporate the UNAA residue into the growing polypeptide chain of the POI. Incorporation occurs in a site-specific manner, e.g., the tRNA^{Pyl} recognizes a codon (e.g., a selector codon such as an amber stop codon) in the mRNA coding for the POI.

As used herein, the term “preferentially acylated” refers to an efficiency of, e.g., about 50% efficient, about 70% efficient, about 75% efficient, about 85% efficient, about 90% efficient, about 95% efficient, or about 99% or more efficient, at which the PylRS acylates the tRNA^{Pyl} with an UNAA compared to an endogenous tRNA or amino acid of a eukaryotic cell. The UNAA is then incorporated into a growing polypeptide chain with high fidelity, e.g., at greater than about 75%, greater than about 80%, greater than about 90%, greater than about 95%, or greater than about 99% or more efficiency for a given codon (e.g., selector codon) that is the reverse complement of the anticodon comprised by the tRNA^{Pyl}.

tRNA^{Pyl}/PylRS pairs suitable in producing a POI according to the present invention may be selected from libraries of mutant tRNA and PylRSs, e.g. based on the results of a library screening. Such selection may be performed analogous to known methods for evolving tRNA/RS pairs described in, e.g., WO 02/085923 and WO 02/06075. To generate a tRNA^{Pyl}/PylRS pair of the invention, one may start from a wild-type or mutant archaeal PylRS that (still) comprises a nuclear localization signal and lacks a NES, and remove the nuclear localization signal and/or introduce a NES prior to or after a suitable tRNA^{Pyl}/PylRS pair is identified.

The term “unnatural amino acid” (abbreviated “UNAA”), as used herein, refers to an amino acid that is not one of the 20 canonical amino acids or selenocysteine or pyrrolysine. The term also refers to amino acid analogs, e.g. compounds which differ from amino acids such that the α-amino group is replaced by a hydroxyl group and/or the carboxylic acid function forms an ester. When translationally incorporated into a polypeptide, said amino acid analogs yield amino acid residues which are different from the amino acid residues corresponding to the 20 canonical amino acids or selenocysteine or pyrrolysine. When UNAAs which are amino acid analogs wherein the carboxylic acid function forms an ester of formula —C(O)—O—R are used for preparing polypeptides in a translation system (such as a eukaryotic cell), it is believed that R is removed in situ, for example enzymatically, in the translation system prior of being incorporated in the POI. Accordingly, R is expediently chosen so as to be compatible with the translation system's ability to convert the UNAA or salt thereof into a form that is recognized and processed by the PylRS of the invention.

UNAAs useful in methods and kits of the present invention have been described in the prior art (for review see e.g. Liu et al., Annu Rev Biochem 83:379-408, 2010, Lemke, ChemBioChem 15:1691-1694, 2014).

As used herein, the term “host cell” or “transformed cell” refers to a cell (or organism) altered to harbor at least one nucleic acid molecule, for instance, a recombinant gene encoding a desired protein or nucleic acid sequence which upon transcription yields a polypeptide for use as described herein. The host cell is a prokaryotic or eukaryotic cell, like a bacterial cell, a fungal cell, a plant cell.an insect cell or mammalian cell. The host cell may contain a recombinant gene which has been integrated into the nuclear or organelle genomes of the host cell. Alternatively, the host may contain the recombinant gene extra-chromosomally.

A particular organism or cell is meant to be “capable of producing a POI” when it produces a POI naturally or when it does not produce said POI naturally but is transformed to produce said POI.

The terms “purified”, “substantially purified,” and “isolated” as used herein refer to the state of being free of other, dissimilar compounds with which a compound of the invention is normally associated in its natural state, so that the “purified”, “substantially purified.” and “isolated” subject comprises at least 0.5%, 1%, 5%, 10%, or 20%, or at least 50% or 75% of the mass, by weight, of a given sample. In one embodiment, these terms refer to the compound of the invention comprising at least 95, 96, 97, 98, 99 or 100%, of the mass, by weight, of a given sample. As used herein, the terms “purified”, “substantially purified.” and “isolated” when referring to a nucleic acid or protein, also refers to a state of purification or concentration different than that which occurs naturally, for example in a prokaryotic or eukaryotic environment, like, for example in a bacterial or fungal cell, or in the mammalian organism, especially human body. Any degree of purification or concentration greater than that which occurs naturally, including (1) the purification from other associated structures or compounds or (2) the association with structures or compounds to which it is not normally associated in said prokaryotic or eukaryotic environment, are within the meaning of “isolated”. The nucleic acid or protein or classes of nucleic acids or proteins, described herein, may be isolated, or otherwise associated with structures or compounds to which they are not normally associated in nature, according to a variety of methods and processes known to those of skill in the art.

In the context of the descriptions provided herein and of the appended claims, the use of “or” means “and/or” unless stated otherwise.

Similarly, “comprise,” “comprises,” “comprising”, “include,” “includes,” and “including” are interchangeable and not intended to be limiting.

It is to be further understood that where descriptions of various embodiments use the term “comprising,” those skilled in the art would understand that in some specific instances, an embodiment can be alternatively described using language “consisting essentially of” or “consisting of.”

The term “about” indicates a potential variation of ±25% of the stated value, in particular ±15%, ±10%, more particularly ±5%, ±2% or ±1%.

The term “substantially” describes a range of values of from about 80 to 100%, such as, for example, 85-99.9%, in particular 90 to 99.9%, more particularly 95 to 99.9%, or 98 to 99.9% and especially 99 to 99.9%.

“Predominantly” refers to a proportion in the range of above 50%, as for example in the range of 51 to 100%, particularly in the range of 75 to 99.9%; more particularly 85 to 98.5%, like 95 to 99%.

If the present disclosure refers to features, parameters and ranges thereof of different degree of preference (including general, not explicitly preferred features, parameters and ranges thereof) then, unless otherwise stated, any combination of two or more of such features, parameters and ranges thereof, irrespective of their respective degree of preference, is encompassed by the disclosure of the present description.

The term “improved activity for utilization of a substrate”, as observed for a particular enzyme or enzyme mutant as described herein refers to an improvement of utilization observed relative to a reference enzyme, in particular the non-mutated parent enzyme or an enzyme mutant differing with respect to number and/or a type of mutations as contained in the mutant showing said improved utilization. A suitable, non-limiting parameter for expressing a change of utilization is the decrease of substrate concentration expressed in %, as for example mole %, or increase of product concentration expressed in %, as for example mole %. A preferred “substrate” in this context is a UNAA as referred to herein

### C. Particular Aspects and Embodiments of the Invention

The present invention relates to the following aspects and particular embodiments thereof:

A first aspect of the invention relates to a modified archaeal pyrrolysyl tRNA synthetase (PylRS), which comprises a combination or set of sequence motifs M1 and M3; in particular, with M1 being closer to the N-terminal end and M3 being closer to the C-terminal end of the amino acid sequence of said modified archaeal PylR; optionally in combination with at least one further sequence motif selected from M2, M4, M5 and M6, and which retains PylRS activity;
wherein
M1, M2, M3, M4, M5 and M6 are arranged in said order within the amino acid sequence of the modified PylRS with M1 being closest to the N-terminal end and M6 being closest to the C-terminal end of the amino acid sequence of said modified archaeal PylRS, and comprise the following sequences (each depicted in the order N-terminus->C-terminus)

M1:
(SEQ ID NO: 1)
LRPMX₁AX₂X₃L(Y/M)X₅X₆(M/V/C)R

analogous to amino acid residues 297-310 of SEQ ID NO: 56 (i.e. PylRS wild type of M. mazei).

M2:
(SEQ ID NO: 2)
HLX₇EFTMX₈NX₉(G/A)X₁₁X₁₂G

analogous to amino acid residues 338-351 of SEQ ID NO: 56 (i.e. PylRS wild type of M. mazei).

M3:
(SEQ ID NO: 3)
VYX₁₃X₁₄TX₁₅D

analogous to amino acid residues 383-389 of SEQ ID NO: 56 (i.e. PylRS wild type of M. mazei).

M4:
(SEQ ID NO: 4)
SX₁₆X₁₇X₁₈GP(R/I/N)X₂₀X₂₁D

analogous to amino acid residues 399-408 of SEQ ID NO: 56 (i.e. PylRS wild type of M. mazei).

M5:
(SEQ ID NO: 5)
X₂₂X₂₃(I/V)X₂₅X₂₆PW

analogous to amino acid residues 411-417 of SEQ ID NO: 56 (i.e. PylRS wild type of M. mazei).

M6:
(SEQ ID NO: 6)
G(A/L/I)GFGLERLL

analogous to amino acid residues 419-428 of SEQ ID NO: 56 (i.e. PylRS wild type of M. mazei);
wherein
amino acid residues X₁ to X₂₆ independently of each other are selected from naturally occurring amino acid residues.

Each of these conserved motifs M1 to M6 contains at least one amino acid residue, which is assumed to participate in the formation of the substrate pocket of the enzyme.

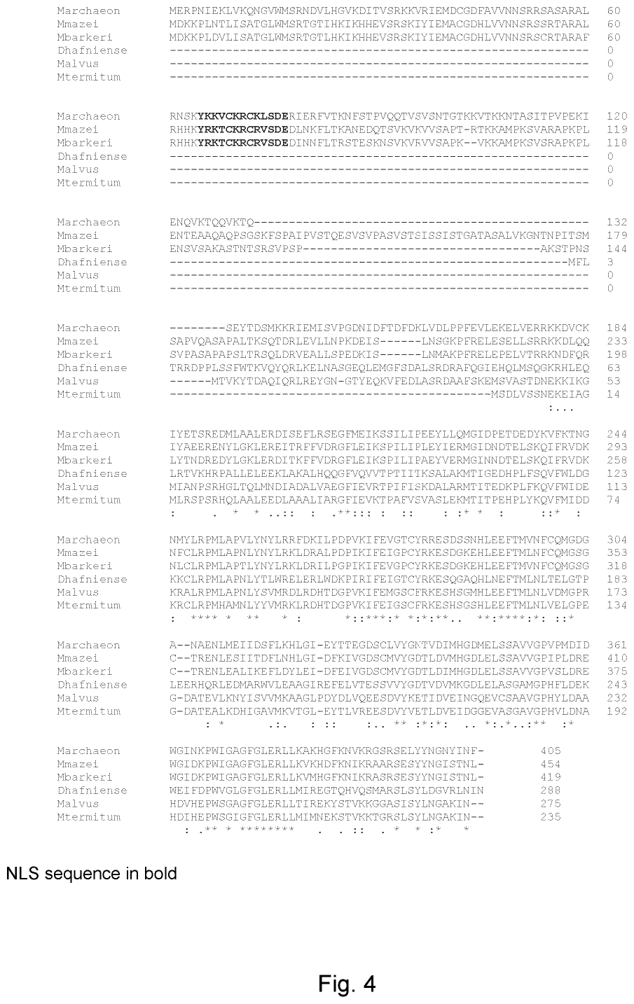

Each intervening sequence motifs linking two neighboring motifs, as well as sequence motifs forming the N-terminal and the C-terminal end sequences may be derived from respective sequence portions of any other wild type or prior art archaeal PylRS. As illustrated by the sequence alignment attached as FIG. 4 respective intervening or N or C-terminal partial sequences can easily be derived from other archaeal PylRS as for example those, of SEQ ID NO: 58, 60, 62, 64 and 66 but not limited thereto. Based on such sequence alignment or any analogous alignment supplements by at least one further PylRS sequence of different archaeal origin, respective further modified sequence motifs may easily be derived as it may be assumed that such less conserved portions do not or not significantly affect synthetase activity.

In a particular embodiment of said modified archaeal PylRS, residues X₁ to X₂₆ independently of each other have the following meanings:
X₁ represents an amino acid residue selected from naturally occurring amino acid residues, in particular L or H,
X₂ represents an amino acid residue selected from naturally occurring amino acid residues, in particular P or M,
X₃ represents an amino acid residue selected from naturally occurring amino acid residues, in particular N, T or V,
X₅ represents an amino acid residue selected from naturally occurring amino acid residues, in particular N, S, T or Y,
X₆ represents an amino acid residue selected from naturally occurring amino acid residues, in particular L, M or W,
X₇ represents an amino acid residue selected from naturally occurring amino acid residues, in particular E or N,
X₈ represents an amino acid residue selected from naturally occurring amino acid residues, in particular V or L,
X₉ represents an amino acid residue selected from naturally occurring amino acid residues, in particular F or L,
X₁₁ represents an amino acid residue selected from naturally occurring amino acid residues, in particular, Q, D or E,
X₁₂ represents an amino acid residue selected from naturally occurring amino acid residues, in particular M or L,
X₁₃ represents an amino acid residue selected from naturally occurring amino acid residues, in particular G, K or V,
X₁₄ represents an amino acid residue selected from naturally occurring amino acid residues, in particular D, E or N,
X₁₅ represents an amino acid residue selected from naturally occurring amino acid residues, in particular L, I or V,
X₁₆ represents an amino acid residue selected from naturally occurring amino acid residues, in particular A or G,
X₁₇ represents an amino acid residue selected from naturally occurring amino acid residues, in particular A or V,
X₁₈ represents an amino acid residue selected from naturally occurring amino acid residues, in particular V or M,
X₂₀ represents an amino acid residue selected from naturally occurring amino acid residues, in particular P, S, Y, F or V,
X₂₁ represents an amino acid residue selected from naturally occurring amino acid residues, in particular L or M,
X₂₂ represents an amino acid residue selected from naturally occurring amino acid residues, in particular W or H,
X₂₃ represents an amino acid residue selected from naturally occurring amino acid residues, in particular G, D or E,
X₂₅ represents an amino acid residue selected from naturally occurring amino acid residues, in particular D, H, F or N,
X₂₆ represents an amino acid residue selected from naturally occurring amino acid residues, in particular K, E or D.

In a further particular embodiment of said first aspect a modified archaeal PylRS is provided, comprising a combination or set of sequence motifs M1, M3 and M2; or M1, M3, M2 and M4; or M1, M3, M2, M4, and M5; or M1, M3, M2, M4, and M6; or M1, M3, M2, M4, M5 andM6.

In a further particular embodiment of said first aspect a modified archaeal PylRS is provided, which is derived from a parental PylRS originating from an archaeal bacterium of the genus Methanosarcina, Methanosarcinaceae, Methanomethylophilus, Desulfitobacterium, and Candidatus Methanoplasma, in particular Methanosarcina.

In a further particular embodiment of said first aspect, a modified archaeal PylRS is provided which is derived from a parental PylRS originating from an archaeal bacterium of the species Methanosarcina mazei (SEQ ID NO:56), Methanosarcina barkeri, (SEQ ID NO:58) Methanosarcinaceae archaeon (SEQ ID NO:60), Methanomethylophilus alvus, (SEQ ID NO:62) Desulfitobacterium hafniense (SEQ ID NO:64), and Candidatus Methanoplasma termitum (SEQ ID NO: 66), in particular Methanosarcina mazei (SEQ ID NO:56).

In a further particular embodiment of said first aspect, a modified archaeal PylRS is provided, which is derived from a parental PylRS having an amino acid sequence selected from SEQ ID NO: 56, 58, 60, 62, 64 and 66, in particular SEQ ID NO: 56, or a functional mutant or fragment thereof, which retains pyrrolysyl tRNA synthetase activity, and which has at least 60% sequence identity, like 65%, 70%, 75%, 80%, 85%, 90%, 91%, 92%, 93%, 94%, 95%, 96%, 97%, 98% or 99%, to the naturally occurring pyrrolysyl tRNA synthetase having an amino acid sequence selected from SEQ ID NO: 56, 58, 60, 62, 64 and 66, and which modified archaeal PylRS comprises a combination of modified sequence motifs M1 and M3; optionally in combination with at least one further sequence motif selected from M2, M4, M5 and M6, each as defined above.

Functional mutants or fragments of a parental PylRS having an amino acid sequence selected from SEQ ID NO: 56, 58, 60, 62, 64 and 66 can easily be derived from a respective sequence alignment, as, for example, one which is depicted in FIG. 4 and which provides information on sequence motifs like M1 to M6 of importance for enzyme function, as well as on intermediary or terminal sequence portions more open to sequence variability, and consequently sequence mutations, as for example conservative amino acid substitution.

In a further particular embodiment of said first aspect, a modified archaeal PylRS is provided, wherein independently of the other motifs within each of the above combinations or sets of motifs
a) the above sequence motif M1 is selected from the following sequences:

M1a:
(SEQ ID NO: 7)
LRPMLAPNLYNYMR
M1b:
(SEQ ID NO: 8)
LRPMLAPTLYNYMR
M1c:
(SEQ ID NO: 9)
LRPMLAPNLYSVMR
M1d:
(SEQ ID NO: 10)
LRPMLAPNLYTLMR
M1e:
(SEQ ID NO: 11)
LRPMLAPVLYNYMR
M1f:
(SEQ ID NO: 12)
LRPMHAMNLYYVMR

b) the above sequence motif M2 is selected from the following sequences:

M2a:
(SEQ ID NO: 13)
HLEEFTMLNFGQMG
M2b:
(SEQ ID NO: 14)
HLEEFTMVNFGQMG
M2c:
(SEQ ID NO: 15)
HLEEFTMLNLGDMG
M2d:
(SEQ ID NO: 16)
HLNEFTMLNLGELG
M2e:
(SEQ ID NO: 17)
HLEEFTMVNFGQMG
M2f:
(SEQ ID NO: 18)
HLEEFTMLNLGELG

c) the above sequence motif M3 is selected from the following sequences

M3a, b:
(SEQ ID NO: 19)
VYGDTLD
M3c:
(SEQ ID NO: 20)
VYKETID
M3d:
(SEQ ID NO: 21)
VYGDTVD
M3e:
(SEQ ID NO: 22)
VYGNTVD
M3f:
(SEQ ID NO: 23)
VYVETLD

d) the above sequence motif M4 is selected from the following sequences

M4a:
(SEQ ID NO: 24)
SAVVGPRPLD
M4b:
(SEQ ID NO: 25)
SAVVGPRSLD
M4c:
(SEQ ID NO: 26)
SAAVGPRYLD
M4d:
(SEQ ID NO: 27)
SGAMGPRFLD
M4e:
(SEQ ID NO: 28)
SAVVGPRPMD
M4f:
(SEQ ID NO: 29)
SGAVGPRVLD

e) the above sequence motif M5 is selected from the following sequences

M5a, b:
(SEQ ID NO: 30)
WGIDKPW
M5c:
(SEQ ID NO: 31)
HDIHEPW
M5d:
(SEQ ID NO: 32)
WEIFDPW
M5e:
(SEQ ID NO: 33)
WGINKPW
M5f:
(SEQ ID NO: 34)
HDIHEPW

f) the above sequence motif M6 is selected from the following sequences

M6a, b, c, d, e, f:
(SEQ ID NO: 35)
GAGFGLERLL

In a further particular embodiment of said first aspect, a modified archaeal PylRS is provided, wherein
a) the above sequence motif M1 is selected from the following sequences:

M1a*:
(SEQ ID NO: 36)
LRPMLAPNLMNYMR
M1b*:
(SEQ ID NO: 37)
LRPMLAPTLMNYMR
M1c*:
(SEQ ID NO: 38)
LRPMLAPNLMSVMR
M1d*:
(SEQ ID NO: 39)
LRPMLAPNLMTLMR
M1e*:
(SEQ ID NO: 40)
LRPMLAPVLMNYMR
M1f*:
(SEQ ID NO: 41)
LRPMHAMNLMYVMR

b) the above sequence motif M2 is selected from the following sequences:

M2a*:
(SEQ ID NO: 42)
HLEEFTMLNFAQMG
M2b*:
(SEQ ID NO: 43)
HLEEFTMVNFAQMG
M2c*:
(SEQ ID NO: 44)
HLEEFTMLNLADMG
M2d*:
(SEQ ID NO: 45)
HLNEFTMLNLAELG
M2e*:
(SEQ ID NO: 46)
HLEEFTMVNFAQMG
M2f*:
(SEQ ID NO: 47)
HLEEFTMLNLAELG

c) the above sequence motif M3 is selected from the following sequences

M3a, b:
(SEQ ID NO: 19)
VYGDTLD
M3c:
(SEQ ID NO: 20)
VYKETID
M3d:
(SEQ ID NO: 21)
VYGDTVD
M3e:
(SEQ ID NO: 22)
VYGNTVD
M3f:
(SEQ ID NO: 23)
VYVETLD

d) the above sequence motif M4 is selected from the following sequences

M4a*:
(SEQ ID NO: 48)
SAVVGPIPLD
M4b*:
(SEQ ID NO: 49)
SAVVGPISLD
M4c*:
(SEQ ID NO: 50)
SAAVGPIYLD
M4d*:
(SEQ ID NO: 51)
SGAMGPIFLD
M4e*:
(SEQ ID NO: 52)
SAVVGPIPMD
M4f*:
(SEQ ID NO: 53)
SGAVGPIVLD

e) the above sequence motif M5 is selected from the following sequences

M5a, b:
(SEQ ID NO: 30)
WGIDKPW
M5c:
(SEQ ID NO: 31)
HDIHEPW
M5d:
(SEQ ID NO: 32)
WEIFDPW
M5e:
(SEQ ID NO: 33)
WGINKPW
M5f:
(SEQ ID NO: 34)
HDIHEPW

f) the above sequence motif M6 is selected from the following sequences

M6a, b, c, d, e, f:
(SEQ ID NO: 35)
GAGFGLERLL

According to further particular embodiments, the following combinations or set of sequence motifs are provided, which are selected from one of the following groups (1) to (5):
(1) M1, M3 and M2:
M1a, M3a and M2a; M1b, M3b and M2b; M1c, M3c and M2c; M1d, M3d and M2d; M1e, M3e and M2e; M1f, M3f and M2f;
M1a*, M3a* and M2a*; M1b*, M3b* and M2b*; M1c*, M3c* and M2c*; M1d*, M3d* and M2d*; M1e*, M3e* and M2e*; M1*f, M3f* and M2f*;
(2) M1, M3, M2 and M4:
M1a, M3a, M2a and M4a; M1b, M3b, M2b and M4b; M1c, M3c, M2c and M4c; M1d, M3d, M2d and M4d; M1e, M3e, M2e and M4e; M1f, M3f, M2f and M4f;
M1a*, M3a*, M2a* and M4a*; M1b*, M3b*, M2b* and M4b*; M1c*, M3c*, M2c* and M4c*; M1d*, M3d*, M2d* and M4d*; M1e*, M3e*, M2e*and M4e*; M1f*, M3f*, M2f* and M4f*;
(3) M1, M3, M2, M4, and M5:
M1a, M3a, M2a, M4a, and M5a; M1b, M3b, M2b, M4b, and M5b; M1c, M3c, M2c, M4c, and M5c; M1d, M3d, M2d, M4d, and M5d; M1e, M3e, M2e, M4e, and M5e; M1f, M3f, M2f, M4f, and M5f;
M1a*, M3a*, M2a*, M4a*, and M5a*; M1b*, M3b*, M2b*, M4b*, and M5b*; M1c*, M3c*, M2c*, M4c*, and M5c*; M1d*, M3d*, M2d*, M4d*, and M5d*; M1e*, M3e*, M2e*, M4e*, and M5e*; M1f*, M3f*, M2f*, M4f*, and M5f*;
(4) M1, M3, M2, M4, and M6:
M1a, M3a, M2a, M4a, and M6a; M1b, M3b, M2b, M4b, and M6b; M1c, M3c, M2c, M4c, and M6c; M1d, M3d, M2d, M4d, and M6d; M1e, M3e, M2e, M4e, and M6e; M1f, M3f, M2f, M4f, and M6f;
M1a*, M3a*, M2a*, M4a*, and M6a*; M1b*, M3b*, M2b*, M4b*, and M6b*; M1c*, M3c*, M2c*, M4c*, and M6c*; M1d*, M3d*, M2d*, M4d*, and M6d*; M1e*, M3e*, M2e*, M4e*, and M6e*; M1f*, M3f*, M2f*, M4f*, and M6f*;
(5) M1, M3, M2, M4, M5 and M6:
M1a, M3a, M2a, M4a, M5a and M6a; M1b, M3b, M2b, M4b, M5b and M6b; M1c, M3c, M2c, M4c, M5c and M6c; M1d, M3d, M2d, M4d, M5d and M6d; M1e, M3e, M2e, M4e, M5e and M6e; M1f, M3f, M2f, M4f, M5f and M6f;
M1a*, M3a*, M2a*, M4a*, M5a* and M6a*; M1b*, M3b*, M2b*, M4b*, M5b* and M6b*; M1c*, M3c*, M2c*, M4c*, M5c* and M6c*; M1d*, M3d*, M2d*, M4d*, M5d* and M6d*; M1e*, M3e*, M2e*, M4e*, M5e* and M6e*; M1f*, M3f*, M2f*, M4f*, M5f* and M6f*;

In a further particular embodiment of said first aspect, a modified archaeal PylRS is provided comprising a combination of sequence motifs M1a, M2a, M3a, M4a, M5a and M6a. A non-limiting example of a PylRS mutant of said type is PylRS A1 (SEQ ID NO:70).

In a further particular embodiment of said first aspect, a modified archaeal PylRS is provided comprising a combination of sequence motifs M1a*, M2a*, M3a, M4a*, M5a and M6a. A non-limiting example of a PylRS mutant of said type is PylRS MMA (SEQ ID NO:72).

In a further particular embodiment of said first aspect, a modified archaeal PylRS is provided, which is
a) PylRS A1 comprising an amino acid sequence of SEQ ID NO: 70; or an amino acid sequence having a degree of sequence identity of at least 60%, like 65%, 70%, 75%, 80%, 85%, 90%, 91%, 92%, 93%, 94%, 95%, 96%, 97%, 98% or 99%, to SEQ ID NO: 70; or a functional fragment thereof retaining PylRS activity; preferably retaining the respective pattern of amino acid residues depicted in Table 2, above; or
b) PylRS MMA comprising an amino acid sequence of SEQ ID NO: 72; or an amino acid sequence having a degree of sequence identity of at least 60%, like 65%, 70%, 75%, 80%, 85%, 90%, 91%, 92%, 93%, 94%, 95%, 96%, 97%, 98% or 99%, to SEQ ID NO: 72; or a functional fragment thereof retaining PylRS activity; preferably retaining the respective pattern of amino acid residues depicted in Table 2, above; or
c) PylRS B11 comprising an amino acid sequence of SEQ ID NO:82; or an amino acid sequence having a degree of sequence identity of at least 60%, like 65%, 70%, 75%, 80%, 85%, 90%, 91%, 92%, 93%, 94%, 95%, 96%, 97%, 98% or 99%, to SEQ ID NO: 82; or a functional fragment thereof retaining PylRS activity; preferably retaining the respective pattern of amino acid residues depicted in Table 2, above;
d) PylRS C11 comprising an amino acid sequence of SEQ ID NO:84; or an amino acid sequence having a degree of sequence identity of at least 60%, like 65%, 70%, 75%, 80%, 85%, 90%, 91%, 92%, 93%, 94%, 95%, 96%, 97%, 98% or 99%, to SEQ ID NO: 84; or a functional fragment thereof retaining PylRS activity; preferably retaining the respective pattern of amino acid residues depicted in Table 2, above;
e) PylRS G3 comprising an amino acid sequence of SEQ ID NO: 86; or an amino acid sequence having a degree of sequence identity of at least 60%, like 65%, 70%, 75%, 80%, 85%, 90%, 91%, 92%, 93%, 94%, 95%, 96%, 97%, 98% or 99%, to SEQ ID NO: 86; or a functional fragment thereof retaining PylRS activity; preferably retaining the respective pattern of amino acid residues depicted in Table 2, above;
f) PylRS H12 comprising an amino acid sequence of SEQ ID NO:88; or an amino acid sequence having a degree of sequence identity of at least 60%, like 65%, 70%, 75%, 80%, 85%, 90%, 91%, 92%, 93%, 94%, 95%, 96%, 97%, 98% or 99%, to SEQ ID NO: 88; or a functional fragment thereof retaining PylRS activity; preferably retaining the respective pattern of amino acid residues depicted in Table 2, above.

In a further particular embodiment of said first aspect, a modified archaeal PylRS is provided which is a functional fusion protein retaining PylRS activity, comprising at least one first protein portion having PylRS activity and a second protein portion covalently linked to the first one with the same or different functionality.

In a further particular embodiment of said first aspect, a modified archaeal PylRS is provided, which shows at least one of the following functional features:
a) Modified substrate profile for non-canonical amino acids (ncAAs) or salts thereof.
b) Improved utilization of at least one bulky ncAA, in particular selected from TCO-E, TCO*A and Boc or salts thereof, each relative to mutant PylRS AF (SEQ ID NO: 68).
c) Improved utilization of at least one bulky ncAA, in particular selected from, TCO*A and Boc or salts thereof, each relative to mutant PylRS AF A1 (SEQ ID NO: 108).

In a further more particular embodiment of said first aspect, a modified archaeal PylRS is provided which shows at least one of the following functional features
a) Improved utilization of at least one bulky ncAA, selected from TCO-E, TCO*A and Boc or salts thereof, each relative to mutant PylRS AF (SEQ ID NO: 68).
b) Improved utilization of at least one bulky ncAA, selected from, TCO*A and Boc or salts thereof, each relative to mutant PylRS AF A1 (SEQ ID NO: 108).

As a non-limiting example of a PylRS mutant meeting such requirement PylRS A1 (SEQ ID NO: 70) may be mentioned.

In a further more particular embodiment of said first aspect, a modified archaeal PylRS is provided, which shows at least the following functional feature:
improved utilization of the bulky ncAA TCO-E or salts thereof relative to mutant PylRS AF (SEQ ID NO: 108).

As a non-limiting example of a PylRS mutant meeting such requirement PylRS MMA (SEQ ID NO:72) may be mentioned.

In a further particular embodiment of said first aspect, a modified archaeal PylRS is provided, that further comprises a nuclear export signal (NES). NES signal sequences ae well known, as described herein above. Each of such known sequence may be considered as candidate for combining said sequence with a PylRS mutant sequence of the invention. The NES may be inserted into or added to the N or C terminal end of the PylRS sequence at any sequence position as long as PylRS activity is not negatively affected. “Added to the N- or C-terminal end” means that NES may be added to the first or the last amino acid of a PylRS mutant sequence or may be inserted in any sequence position in a sequence portion corresponding to the respective 30, 20, 10 or 5 terminal amino acid residues of the enzymes N or C-terminus, respectively.

In particular, said NES comprises an amino acid sequence SEQ ID NO: 54, which is, in particular added to the N-terminal end of the enzyme.

Non-limiting examples of NES-modified PylRS mutants of the present invention have an amino acid sequence selected from SEQ ID NO: 78, 80, 90, 92, 94 and 96; or an amino acid sequence having a degree of sequence identity of at least 60%, like 65%, 70%, 75%, 80%, 85%, 90%, 91%, 92%, 93%, 94%, 95%, 96%, 97%, 98% or 99%, to SEQ ID NO: 78, 80, 90, 92, 94 and 96, respectively and retaining said NES motif.

In a further particular embodiment of said first aspect, a modified archaeal PylRS is provided, which is
a) NES PylRS A1 comprising an amino acid sequence of SEQ ID NO: 78; or an amino acid sequence having a degree of sequence identity of at least 60%, like 65%, 70%, 75%, 80%, 85%, 90%, 91%, 92%, 93%, 94%, 95%, 96%, 97%, 98% or 99%, to SEQ ID NO: 70; or a functional fragment thereof retaining PylRS activity; preferably retaining the respective pattern of amino acid residues depicted in Table 2, above; or
b) NES PylRS MMA comprising an amino acid sequence of SEQ ID NO:80; or an amino acid sequence having a degree of sequence identity of at least 60%, like 65%, 70%, 75%, 80%, 85%, 90%, 91%, 92%, 93%, 94%, 95%, 96%, 97%, 98% or 99%, to SEQ ID NO: 72; or a functional fragment thereof retaining PylRS activity; preferably retaining the respective pattern of amino acid residues depicted in Table 2, above; or
c) NES PylRS B11 comprising an amino acid sequence of SEQ ID NO:90; or an amino acid sequence having a degree of sequence identity of at least 60%, like 65%, 70%, 75%, 80%, 85%, 90%, 91%, 92%, 93%, 94%, 95%, 96%, 97%, 98% or 99%, to SEQ ID NO: 82; or a functional fragment thereof retaining PylRS activity; preferably retaining the respective pattern of amino acid residues depicted in Table 2, above;
d) NES PylRS C11 comprising an amino acid sequence of SEQ ID NO:92; or an amino acid sequence having a degree of sequence identity of at least 60%, like 65%, 70%, 75%, 80%, 85%, 90%, 91%, 92%, 93%, 94%, 95%, 96%, 97%, 98% or 99%, to SEQ ID NO: 84; or a functional fragment thereof retaining PylRS activity; preferably retaining the respective pattern of amino acid residues depicted in Table 2, above;
e) NES PylRS G3 comprising an amino acid sequence of SEQ ID NO: 94; or an amino acid sequence having a degree of sequence identity of at least 60%, like 65%, 70%, 75%, 80%, 85%, 90%, 91%, 92%, 93%, 94%, 95%, 96%, 97%, 98% or 99%, to SEQ ID NO: 86; or a functional fragment thereof retaining PylRS activity; preferably retaining the respective pattern of amino acid residues depicted in Table 2, above;
f) NES PylRS H12 comprising an amino acid sequence of SEQ ID NO:96; or an amino acid sequence having a degree of sequence identity of at least 60%, like 65%, 70%, 75%, 80%, 85%, 90%, 91%, 92%, 93%, 94%, 95%, 96%, 97%, 98% or 99%, to SEQ ID NO: 88; or a functional fragment thereof retaining PylRS activity; preferably retaining the respective pattern of amino acid residues depicted in Table 2, above.

A second aspect of the present invention relates to modified, in particular recombinant, polynucleotides or nucleic acids, comprising a nucleotide sequence encoding at least one of the modified archaeal pyrrolysyl tRNA synthetases or functional fragments as defined above according to said first aspect; as well as the corresponding complementary polynucleotides; as well as fragments thereof hybridizing under stringent conditions as herein defined to anyone of the above mention polynucleotides.

Particular non-limiting examples of coding polynucleotides comprise a nucleotide sequence selected from SEQ ID NO: 69, 71, 77, 79, 81, 83, 85, 87, 89, 91, 93 and 95 or a nucleotide sequence having a degree of sequence identity of at least 60%, like 65%, 70%, 75%, 80%, 85%, 90%, 91%, 92%, 93%, 94%, 95%, 96%, 97%, 98% or 99%, to a nucleotide sequence selected from SEQ ID NO: 69, 71, 77, 79, 81, 83, 85, 87, 89, 91, 93 and 95, while still encoding a functional PylRS mutant of the present invention.

As further examples there may be mentioned nucleic acid sequences encoding anyone of the above mentioned motifs according to SEQ ID NO: 7 to 53 or encoding PylRS mutants comprising at least one of such motif, or more particularly, combinations of such motifs as disclosed above.

In a particular embodiment, said polynucleotide further encodes a tRNA^{Pyl} (SEQ ID NO: 113, optionally without the terminal CCA triplett), wherein the tRNA^{Pyl} is a tRNA that can be acylated by said pyrrolysyl tRNA synthase mutant of the invention encoded, as defined above.

In a further particular embodiment of said second aspect, a combination of polynucleotides is provided, comprising at least one polynucleotide coding said PylRS mutant of the invention and at least one polynucleotide encoding said tRNA^{Pyl}.

In a further particular embodiment, the anticodon of the tRNA^{Pyl} is the reverse complement of a codon that is being selected from stop codons, four base codons and rare codons.

The polynucleotides of the invention as well as the tRNA^{Pyl}- and/or POI-encoding polynucleotides used in the context of the present invention are preferably expression systems like expression cassettes.

The present invention also provides vectors suitable for transfecting a eukaryotic cell and allowing for the expression of the encoded PylRS mutant, tRNA^{Pyl} and/or POI, respectively, in said cell.

In particular, such vectors are expression vector comprising

a recombinant expression system as defined above; or one of the above nucleic acids or the reverse complement thereof; or

An expression vector of the invention is a prokaryotic vector, viral vector, a eukaryotic vector, or a plasmid.

A third aspect of the present invention relates to a eukaryotic cell comprising:
(a) a polynucleotide sequence encoding at least one of the pyrrolysyl tRNA synthetase mutants of the above first aspect, and
(b) a tRNA that can be acylated by the pyrrolysyl tRNA synthase mutant encoded by the sequence of (a), or a polynucleotide sequence encoding such tRNA.

In particular, said eukaryotic cell is a mammalian cell.

The present invention in particular provides a mammalian cell capable of expressing a PylRS mutant of the invention. In particular, the present invention provides a mammalian cell comprising a polynucleotide or combination of polynucleotides, wherein said polynucleotide(s) encode(s) the PylRS mutant of the invention and a tRNA^{Pyl}, wherein the tRNA^{Pyl} is a tRNA that can be acylated (preferably selectively) by the PylRS. Expediently, the mammalian cell of the invention is capable of expressing both the tRNA^{Pyl} and the PylRS mutant of the invention, wherein the PylRS mutant is capable of acylating the tRNA^{Pyl} (preferably selectively) with an amino acid, e.g. with an UNAA.

A fourth aspect of the present invention relates to a method for preparing a POI comprising one or more than one unnatural amino acid residue, wherein the method comprises:
(a) providing the eukaryotic cell comprising:
   (i) a pyrrolysyl tRNA synthetase mutant of the above first aspect,
   (ii) a tRNA (tRNA^{Pyl}),
   (iii) an unnatural amino acid or a salt thereof, and
   (iv) a polynucleotide encoding the POI, wherein any position of the POI occupied by an unnatural amino acid residue is encoded by a codon that is the reverse complement of the anticodon comprised by the tRNA^{Pyl}; and
      wherein the pyrrolysyl tRNA synthetase mutant (i) is capable of acylating the tRNA^{Pyl} (ii) with the unnatural amino acid or salt (iii); and
(b) allowing for translation of the polynucleotide (iv) by the eukaryotic cell, thereby producing the POI.

A fifth aspect of the present invention relates to a method for preparing a polypeptide conjugate comprising:
(a) preparing a POI comprising one or more than one unnatural amino acid residue using the method according to the fourth aspect; and
(b) reacting the POI with one or more than one conjugation partner molecule such that the conjugation partner molecules bind covalently to the unnatural amino acid residue(s) of the POI.

A POI of the present invention having incorporated into its amino acid sequence at least one ncAA in a particular embodiment is utilized to form a “targeting agent”.

The primary object of such targeting agent is the formation of a covalent or noncovalent linkage with a particular “target”. A secondary object of the targeting agent is the targeted transport of a “payload molecule” to said target. In order to achieve said second object the POI has to be combined (reversibly or irreversibly) with at least one payload molecule. For this purpose said POI has to be functionalised by introducing said at least one ncAA. The functionalized POI carrying said at least one ncAA, may then be linked to said at least one payload molecule through bioconjugation via said ncAA residue. Said ncAA is reactive with the payload molecule which in turn carries a corresponding moiety reactive with said ncAA residue of the POI. The thus obtained bioconjugate, i.e. the targeting agent, allows the transfer of the payload molecule to the intended target.

Without being limited thereto, particular examples of ncAAs to be incorporated into a POI are bulky ncAAs referred to herein. More particularly, such residues are TCO*A, TCO-E, Boc, and SCO* or salts thereof. Particular, non-limiting examples of suitable types of POIs, of potential targets, of potential payloads and potential reactive groups suitable for bioconjugation with such ncAAs are described herein below.

In particular, the POI is an immunoglobulin, in particular antibody molecule, more particularly a monoclonal antibody or a functional antigen binding derivative or fragment thereof, carrying at least one ncAA in its amino acid sequence.

In another particular embodiment, said target is a tumor-associated target, and the payload molecule is an anti-tumor agent.

A sixth aspect of the present invention relates to kits that can be used in methods for preparing UNAA-residue containing POIs or conjugates thereof as described herein.

Such kits may comprise at least one unnatural amino acid, or a salt thereof; and/or a polynucleotide or a combination of polynucleotides, as defined above according to the second aspect of the invention or a eukaryotic cell according to the third aspect of the invention: wherein the encoded archaeal PylRS is capable of acylating the tRNA^{Pyl} with the unnatural amino acid or salt thereof.

For example, such kits comprise a polynucleotide encoding a PylRS mutant of the present invention or a eukaryotic cell capable of expressing such PylRS and further comprise at least one unnatural amino acid, or a salt thereof, which can be used for acylating a tRNA in a reaction catalyzed by said PylRS.

Particular amino acid sequences of PylRS of the invention referred to herein are given below.

Particular example sequences of PylRS mutants of the invention:

Any NES sequence is twice underlined

Mutations are in bold letters and underlined; the numbering of mutation positions relative to the wild type sequence (SEQ ID NO: 56)

Particular PylRS mutants of the invention are derivable from M. mazei wildtype PylRS, or an enzymatically active fragment thereof.

The amino acid sequence of wild type M. mazei PylRS is set forth in SEQ ID NO:56.

| | |
|---|---|
| MDKKPLNTLISATGLWMSRTGTIHKIKHHEVSRSKIYIEMACGDHLVVNNSRSSRTARAL | 60 |
| RHHKYRKTCKRCRVSDEDLNKFLTKANEDQTSVKVKVVSAPTRTKKAMPKSVARAPKPLE | 120 |
| NTEAAQAQPSGSKFSPAIPVSTQESVSVPASVSTSISSISTGATASALVKGNTNPITSMS | 180 |
| APVQASAPALTKSQTDRLEVLLNPKDEISLNSGKPFRELESELLSRRKKDLQQIYAEERE | 240 |
| NYLGKLEREITRFFVDRGFLEIKSPILIPLEYIERMGIDNDTELSKQIFRVDKNFCLRPM | 300 |
| LAPNLYNYLRKLDRALPDPIKIFEIGPCYRKESDGKEHLEEFTMLNFCQMGSGCTRENLE | 360 |
| SIITDFLNHLGIDFKIVGDSCMVYGDTLDVMHGDLELSSAVVGPIPLDREWGIDKPWIGA | 420 |
| GFGLERLLKVKHDFKNIKRAARSESYYNGISTNL | 454 |

In the following PylRS mutant sequences the NES sequence is depicted double underlined. Mutant amino acid residues a stated in bold and underlined letters.

NES-PylRS A1: L309M, C348G, I405R
(SEQ ID NO: 78)
MACPVPLQLPPLERLTLDDKKPLNTLISATGLWMSRTGTIHKIKHHEVSRSKIYIEMACG
DHLVVNNSRSSRTARALRHHKYRKTCKRCRVSDEDLNKFLTKANEDQTSVKVKVVSA
PTRTKKAMPKSVARAPKPLENTEAAQAQPSGSKFSPAIPVSTQESVSVPASVSTSISSIST
GATASALVKGNTNPITSMSAPVQASAPALTKSQTDRLEVLLNPKDEISLNSGKPFRELES
ELLSRRKKDLQQIYAEERENYLGKLEREITRFFVDRGFLEIKSPILIPLEYIERMGIDNDTE
LSKQIFRVDKNFCLRPMLAPNLYNYMRKLDRALPDPIKIFEIGPCYRKESDGKEHLEEFT
MLNFGQMGSGCTRENLESIITDFLNHLGIDFKIVGDSCMVYGDTLDVMHGDLELSSAV
VGPRPLDREWGIDKPWIGAGFGLERLLKVKHDFKNIKRAARSESYYNGISTNL
PylRS A1: L309M, C348G, I405R
(SEQ ID NO: 70)
MDKKPLNTLISATGLWMSRTGTIHKIKHHEVSRSKIYIEMACGDHLVVNNSRSSRTARA
LRHHKYRKTCKRCRVSDEDLNKFLTKANEDQTSVKVKVVSAPTRTKKAMPKSVARAP
KPLENTEAAQAQPSGSKFSPAIPVSTQESVSVPASVSTSISSISTGATASALVKGNTNPITS
MSAPVQASAPALTKSQTDRLEVLLNPKDEISLNSGKPFRELESELLSRRKKDLQQIYAEE
RENYLGKLEREITRFFVDRGFLEIKSPILIPLEYIERMGIDNDTELSKQIFRVDKNFCLRPM
LAPNLYNYMRKLDRALPDPIKIFEIGPCYRKESDGKEHLEEFTMLNFGQMGSGCTRENL
ESIITDFLNHLGIDFKIVGDSCMVYGDTLDVMHGDLELSSAVVGPRPLDREWGIDKPWI
GAGFGLERLLKVKHDFKNIKRAARSESYYNGISTNL
NES-PylRS MMA: Y306M, L309M, C348A,
(SEQ ID NO: 80)
MACPVPLQLPPLERLTLDDKKPLNTLISATGLWMSRTGTIHKIKHHEVSRSKIYIEMACG
DHLVVNNSRSSRTARALRHHKYRKTCKRCRVSDEDLNKFLTKANEDQTSVKVKVVSA
PTRTKKAMPKSVARAPKPLENTEAAQAQPSGSKFSPAIPVSTQESVSVPASVSTSISSIST
GATASALVKGNTNPITSMSAPVQASAPALTKSQTDRLEVLLNPKDEISLNSGKPFRELES
ELLSRRKKDLQQIYAEERENYLGKLEREITRFFVDRGFLEIKSPILIPLEYIERMGIDNDTE
LSKQIFRVDKNFCLRPMLAPNLMNYMRKLDRALPDPIKIFEIGPCYRKESDGKEHLEEFT
MLNFAQMGSGCTRENLESIITDFLNHLGIDFKIVGDSCMVYGDTLDVMHGDLELSSAVV
GPIPLDREWGIDKPWIGAGFGLERLLKVKHDFKNIKRAARSESYYNGISTNL
PylRS MMA: Y306M, L309M, C348A,
(SEQ ID NO: 72)
MDKKPLNTLISATGLWMSRTGTIHKIKHHEVSRSKIYIEMACGDHLVVNNSRSSRTARA
LRHHKYRKTCKRCRVSDEDLNKFLTKANEDQTSVKVKVVSAPTRTKKAMPKSVARAP
KPLENTEAAQAQPSGSKFSPAIPVSTQESVSVPASVSTSISSISTGATASALVKGNTNPITS
MSAPVQASAPALTKSQTDRLEVLLNPKDEISLNSGKPFRELESELLSRRKKDLQQIYAEE
RENYLGKLEREITRFFVDRGFLEIKSPILIPLEYIERMGIDNDTELSKQIFRVDKNFCLRPM
LAPNLMNYMRKLDRALPDPIKIFEIGPCYRKESDGKEHLEEFTMLNFAQMGSGCTRENL
ESIITDFLNHLGIDFKIVGDSCMVYGDTLDVMHGDLELSSAVVGPIPLDREWGIDKPWIG
AGFGLERLLKVKHDFKNIKRAARSESYYNGISTNL

### D. Further Embodiments

### 1. Polypeptides of the Invention

In this context, the following definitions apply:

“Functional mutants” of herein described polypeptides include the “functional equivalents” of such polypeptides as defined below.

An “enzyme”, “protein” or “polypeptide” as described herein can be a native or recombinantly produced enzyme, protein or polypeptide it may be the wild type enzyme, protein or polypeptide or genetically modified by suitable mutations or by C- and/or N-terminal amino acid sequence extensions, like His-tag containing sequences. The enzyme, protein or polypeptide basically can be mixed with cellular, for example protein impurities, but particularly is in pure form. Suitable methods of detection are described for example in the experimental section given below or are known from the literature.

A “pure form” or a “pure” or “substantially pure” enzyme, protein or polypeptide is to be understood according to the invention as an enzyme, protein or polypeptide with a degree of purity above 80, particularly above 90, especially above 95, and quite particularly above 99 wt %, relative to the total protein content, determined by means of usual methods of detecting proteins, for example the biuret method or protein detection according to Lowry et al. (cf. description in R. K. Scopes, Protein Purification, Springer Verlag, New York, Heidelberg, Berlin (1982)).

“Proteinogenic” amino acids comprise in particular (single-letter code): G, A, V, L, I, F, P, M, W, S, T, C, Y, N, Q, D, E, K, R and H.

The generic terms “polypeptide” or “peptide”, which may be used interchangeably, refer to a natural or synthetic linear chain or sequence of consecutive, peptidically linked amino acid residues, comprising about 10 to up to more than 1.000 residues. Short chain polypeptides with up to 30 residues are also designated as “oligopeptides”.

The term “protein” refers to a macromolecular structure consisting of one or more polypeptides. The amino acid sequence of its polypeptide(s) represents the “primary structure” of the protein. The amino acid sequence also predetermines the “secondary structure” of the protein by the formation of special structural elements, such as alpha-helical and beta-sheet structures formed within a polypeptide chain. The arrangement of a plurality of such secondary structural elements defines the “tertiary structure” or spatial arrangement of the protein. If a protein comprises more than one polypeptide chains said chains are spatially arranged forming the “quaternary structure” of the protein. A correct spatial arrangement or “folding” of the protein is prerequisite of protein function. Denaturation or unfolding destroys protein function. If such destruction is reversible, protein function may be restored by refolding.

A typical protein function referred to herein is an “enzyme function”, i.e. the protein acts as biocatalyst on a substrate, for example a chemical compound, and catalyzes the conversion of said substrate to a product. An enzyme may show a high or low degree of substrate and/or product specificity.

A “polypeptide” referred to herein as having a particular “activity” thus implicitly refers to a correctly folded protein showing the indicated activity, as for example a specific enzyme activity.

Thus, unless otherwise indicated the term “polypeptide” also encompasses the terms “protein” and “enzyme”.

Similarly, the term “polypeptide fragment” encompasses the terms “protein fragment” and “enzyme fragment”.

The term “isolated polypeptide” refers to an amino acid sequence that is removed from its natural environment by any method or combination of methods known in the art and includes recombinant, biochemical and synthetic methods.

The present invention also relates to “functional equivalents” (also designated as “analogs” or “functional mutations”) of the polypeptides specifically described herein.

For example, “functional equivalents” refer to polypeptides, which, in a test used for determining enzymatic NHase activity display at least a 1 to 10%, or at least 20%, or at least 50%, or at least 75%, or at least 90% higher or lower activity, as that of the polypeptides specifically described herein.

“Functional equivalents”, according to the invention, also cover particular mutants, which, in at least one sequence position of an amino acid sequences stated herein, have an amino acid that is different from that concretely stated one, but nevertheless possess one of the aforementioned biological activities, as for example enzyme activity. “Functional equivalents” thus comprise mutants obtainable by one or more, like 1 to 20, in particular 1 to 15 or 5 to 10 amino acid additions, substitutions, in particular conservative substitutions, deletions and/or inversions, where the stated changes can occur in any sequence position, provided they lead to a mutant with the profile of properties according to the invention. Functional equivalence is in particular also provided if the activity patterns coincide qualitatively between the mutant and the unchanged polypeptide, i.e. if, for example, interaction with the same agonist or antagonist or substrate, however at a different rate, (i.e. expressed by a EC₅₀ or IC₅₀ value or any other parameter suitable in the present technical field) is observed. Examples of suitable (conservative) amino acid substitutions are shown in the following Table 3:

TABLE 3
Examples of conservative amino acid substitutions

| Original residue | Examples of substitution |
|---|---|
| Ala | Ser |
| Arg | Lys |
| Asn | Gln; His |
| Asp | Glu |
| Cys | Ser |
| Gln | Asn |
| Glu | Asp |
| Gly | Pro |
| His | Asn; Gln |
| Ile | Leu; Val |
| Leu | Ile; Val |
| Lys | Arg; Gln; Glu |
| Met | Leu; Ile |
| Phe | Met; Leu; Tyr |
| Ser | Thr |
| Thr | Ser |
| Trp | Tyr |
| Tyr | Trp; Phe |
| Val | Ile; Leu |

“Functional equivalents” in the above sense are also “precursors” of the polypeptides described herein, as well as “functional derivatives” and “salts” of the polypeptides.

“Precursors” are in that case natural or synthetic precursors of the polypeptides with or without the desired biological activity.

The expression “salts” means salts of carboxyl groups as well as salts of acid addition of amino groups of the protein molecules according to the invention. Salts of carboxyl groups can be produced in a known way and comprise inorganic salts, for example sodium, calcium, ammonium, iron and zinc salts, and salts with organic bases, for example amines, such as triethanolamine, arginine, lysine, piperidine and the like. Salts of acid addition, for example salts with inorganic acids, such as hydrochloric acid or sulfuric acid and salts with organic acids, such as acetic acid and oxalic acid, are also covered by the invention.

“Functional derivatives” of polypeptides according to the invention can also be produced on functional amino acid side groups or at their N-terminal or C-terminal end using known techniques. Such derivatives comprise for example aliphatic esters of carboxylic acid groups, amides of carboxylic acid groups, obtainable by reaction with ammonia or with a primary or secondary amine; N-acyl derivatives of free amino groups, produced by reaction with acyl groups; or O-acyl derivatives of free hydroxyl groups, produced by reaction with acyl groups.

“Functional equivalents” naturally also comprise polypeptides that can be obtained from other organisms, as well as naturally occurring variants. For example, areas of homologous sequence regions can be established by sequence comparison, and equivalent polypeptides can be determined on the basis of the concrete parameters of the invention.

“Functional equivalents” also comprise “fragments”, like individual domains or sequence motifs, of the polypeptides according to the invention, or N- and or C-terminally truncated forms, which may or may not display the desired biological function. Particularly such “fragments” retain the desired biological function at least qualitatively.

“Functional equivalents” are, moreover, fusion proteins, which have one of the polypeptide sequences stated herein or functional equivalents derived there from and at least one further, functionally different, heterologous sequence in functional N-terminal or C-terminal association (i.e. without substantial mutual functional impairment of the fusion protein parts). Non-limiting examples of these heterologous sequences are e.g. signal peptides, histidine anchors or enzymes.

“Functional equivalents” which are also comprised in accordance with the invention are homologs to the specifically disclosed polypeptides. These have at least 50%, 55%, or 60%, in particular at least 75%, more particular at least 80 or 85%, such as, for example, 90, 91, 92, 93, 94, 95, 96, 97, 98 or 99%, homology (or identity) to one of the specifically disclosed amino acid sequences, calculated by the algorithm of Pearson and Lipman, Proc. Natl. Acad, Sci. (USA) 85 (8), 1988, 2444-2448. A homology or identity, expressed as a percentage, of a homologous polypeptide according to the invention means in particular an identity, expressed as a percentage, of the amino acid residues based on the total length of one of the amino acid sequences described specifically herein.

The identity data, expressed as a percentage, may also be determined with the aid of BLAST alignments, algorithm blastp (protein-protein BLAST), or by applying the Clustal settings specified herein below.

In the case of a possible protein glycosylation, “functional equivalents” according to the invention comprise polypeptides as described herein in deglycosylated or glycosylated form as well as modified forms that can be obtained by altering the glycosylation pattern.

Functional equivalents or homologues of the polypeptides according to the invention can be produced by mutagenesis, e.g. by point mutation, lengthening or shortening of the protein or as described in more detail below.

Functional equivalents or homologs of the polypeptides according to the invention can be identified by screening combinatorial databases of mutants, for example shortening mutants. For example, a variegated database of protein variants can be produced by combinatorial mutagenesis at the nucleic acid level, e.g. by enzymatic ligation of a mixture of synthetic oligonucleotides. There are a great many methods that can be used for the production of databases of potential homologues from a degenerated oligonucleotide sequence. Chemical synthesis of a degenerated gene sequence can be carried out in an automatic DNA synthesizer, and the synthetic gene can then be ligated in a suitable expression vector. The use of a degenerated genome makes it possible to supply all sequences in a mixture, which code for the desired set of potential protein sequences. Methods of synthesis of degenerated oligonucleotides are known to a person skilled in the art.

In the prior art, several techniques are known for the screening of gene products of combinatorial databases, which were produced by point mutations or shortening, and for the screening of cDNA libraries for gene products with a selected property. These techniques can be adapted for the rapid screening of the gene banks that were produced by combinatorial mutagenesis of homologues according to the invention. The techniques most frequently used for the screening of large gene banks, which are based on a high-throughput analysis, comprise cloning of the gene bank in expression vectors that can be replicated, transformation of the suitable cells with the resultant vector database and expression of the combinatorial genes in conditions in which detection of the desired activity facilitates isolation of the vector that codes for the gene whose product was detected. Recursive Ensemble Mutagenesis (REM), a technique that increases the frequency of functional mutants in the databases, can be used in combination with the screening tests, in order to identify homologues.

The polypeptides of the invention include all active forms, including active subsequences, e.g., catalytic domains or active sites, of an enzyme of the invention. In one aspect, the invention provides catalytic domains or active sites as set forth below. In one aspect, the invention provides a peptide or polypeptide comprising or consisting of an active site domain as predicted through use of a database such as Pfam (http://pfam.wustl.edu/hmmsearch.shtml) (which is a large collection of multiple sequence alignments and hidden Markov models covering many common protein families, The Pfam protein families database, A. Bateman, E. Birney, L. Cerruti, R. Durbin. L. Etwiller, S. R. Eddy, S. Griffiths-Jones, K. L. Howe, M. Marshall, and E. L. L. Sonnhammer, Nucleic Acids Research, 30 (1): 276-280, 2002) or equivalent, as for example InterPro and SMART databases (http://www.ebi.ac.uk/interpro/scan.html, http://smart.embl-heidelberg.de/).

The invention also encompasses “polypeptide variant” having the desired activity, wherein the variant polypeptide is selected from an amino acid sequence having at least 50%, 55%, 60%, 65%, 70%, 75%, 80%, 85%, 90%, 91%, 92%, 93%, 94%, 95%, 96%, 97%, 98% or 99%, sequence identity to a specific, in particular natural, amino acid sequence as referred to by a specific SEQ ID NO and contains at least one substitution modification relative said SEQ ID NO.

### 2. Nucleic Acids and Constructs

### 2.1 Nucleic Acids

In this context the following definitions apply:

The terms “nucleic acid sequence,” “nucleic acid,” “nucleic acid molecule” and “polynucleotide” are used interchangeably meaning a sequence of nucleotides. A nucleic acid sequence may be a single-stranded or double-stranded deoxyribonucleotide, or ribonucleotide of any length, and include coding and non-coding sequences of a gene, exons, introns, sense and anti-sense complimentary sequences, genomic DNA, cDNA, miRNA, siRNA, mRNA, rRNA, tRNA, recombinant nucleic acid sequences, isolated and purified naturally occurring DNA and/or RNA sequences, synthetic DNA and RNA sequences, fragments, primers and nucleic acid probes. The skilled artisan is aware that the nucleic acid sequences of RNA are identical to the DNA sequences with the difference of thymine (T) being replaced by uracil (U). The term “nucleotide sequence” should also be understood as comprising a polynucleotide molecule or an oligonucleotide molecule in the form of a separate fragment or as a component of a larger nucleic acid.

An “isolated nucleic acid” or “isolated nucleic acid sequence” relates to a nucleic acid or nucleic acid sequence that is in an environment different from that in which the nucleic acid or nucleic acid sequence naturally occurs and can include those that are substantially free from contaminating endogenous material.

The term “naturally-occurring” as used herein as applied to a nucleic acid refers to a nucleic acid that is found in a cell of an organism in nature and which has not been intentionally modified by a human in the laboratory.

A “fragment” of a polynucleotide or nucleic acid sequence refers to contiguous nucleotides that is particularly at least 15 bp, at least 30 bp, at least 40 bp, at least 50 bp and/or at least 60 bp in length of the polynucleotide of an embodiment herein. Particularly the fragment of a polynucleotide comprises at least 25, more particularly at least 50, more particularly at least 75, more particularly at least 100, more particularly at least 150, more particularly at least 200, more particularly at least 300, more particularly at least 400, more particularly at least 500, more particularly at least 600, more particularly at least 700, more particularly at least 800, more particularly at least 900, more particularly at least 1000 contiguous nucleotides of the polynucleotide of an embodiment herein. Without being limited, the fragment of the polynucleotides herein may be used as a PCR primer, and/or as a probe, or for anti-sense gene silencing or RNAi.

As used herein, the term “hybridization” or hybridizes under certain conditions is intended to describe conditions for hybridization and washes under which nucleotide sequences that are significantly identical or homologous to each other remain bound to each other. The conditions may be such that sequences, which are at least about 70%, such as at least about 80%, and such as at least about 85%, 90%, or 95% identical, remain bound to each other. Definitions of low stringency, moderate, and high stringency hybridization conditions are provided herein below. Appropriate hybridization conditions can also be selected by those skilled in the art with minimal experimentation as exemplified in Ausubel et al. (1995, Current Protocols in Molecular Biology, John Wiley & Sons, sections 2, 4, and 6). Additionally, stringency conditions are described in Sambrook et al. (1989, Molecular Cloning: A Laboratory Manual, 2nd ed., Cold Spring Harbor Press, chapters 7, 9, and 11).

“Recombinant nucleic acid sequences” are nucleic acid sequences that result from the use of laboratory methods (for example, molecular cloning) to bring together genetic material from more than on source, creating or modifying a nucleic acid sequence that does not occur naturally and would not be otherwise found in biological organisms.

“Recombinant DNA technology” refers to molecular biology procedures to prepare a recombinant nucleic acid sequence as described, for instance, in Laboratory Manuals edited by Weigel and Glazebrook, 2002, Cold Spring Harbor Lab Press; and Sambrook et al., 1989, Cold Spring Harbor, NY, Cold Spring Harbor Laboratory Press.

The term “gene” means a DNA sequence comprising a region, which is transcribed into a RNA molecule, e.g., an mRNA in a cell, operably linked to suitable regulatory regions, e.g., a promoter. A gene may thus comprise several operably linked sequences, such as a promoter, a 5′ leader sequence comprising. e.g., sequences involved in translation initiation, a coding region of cDNA or genomic DNA, introns, exons, and/or a 3′non-translated sequence comprising, e.g., transcription termination sites.

“Polycistronic” refers to nucleic acid molecules, in particular mRNAs, that can encode more than one polypeptide separately within the same nucleic acid molecule

A “chimeric gene” refers to any gene which is not normally found in nature in a species, in particular, a gene in which one or more parts of the nucleic acid sequence are present that are not associated with each other in nature. For example the promoter is not associated in nature with part or all of the transcribed region or with another regulatory region. The term “chimeric gene” is understood to include expression constructs in which a promoter or transcription regulatory sequence is operably linked to one or more coding sequences or to an antisense, i.e., reverse complement of the sense strand, or inverted repeat sequence (sense and antisense, whereby the RNA transcript forms double stranded RNA upon transcription). The term “chimeric gene” also includes genes obtained through the combination of portions of one or more coding sequences to produce a new gene.

A “3′ UTR” or “3′ non-translated sequence” (also referred to as “3′ untranslated region,” or “3′end”) refers to the nucleic acid sequence found downstream of the coding sequence of a gene, which comprises, for example, a transcription termination site and (in most, but not all eukaryotic mRNAs) a polyadenylation signal such as AAUAAA or variants thereof. After termination of transcription, the mRNA transcript may be cleaved downstream of the polyadenylation signal and a poly(A) tail may be added, which is involved in the transport of the mRNA to the site of translation, e.g., cytoplasm.

The term “primer” refers to a short nucleic acid sequence that is hybridized to a template nucleic acid sequence and is used for polymerization of a nucleic acid sequence complementary to the template.

The term “selectable marker” refers to any gene which upon expression may be used to select a cell or cells that include the selectable marker. Examples of selectable markers are described below. The skilled artisan will know that different antibiotic, fungicide, auxotrophic or herbicide selectable markers are applicable to different target species.

The invention also relates to nucleic acid sequences that code for polypeptides as defined herein.

In particular, the invention also relates to nucleic acid sequences (single-stranded and double-stranded DNA and RNA sequences, e.g. cDNA, genomic DNA and mRNA), coding for one of the above polypeptides and their functional equivalents, which can be obtained for example using artificial nucleotide analogs.

The invention relates both to isolated nucleic acid molecules, which code for polypeptides according to the invention or biologically active segments thereof, and to nucleic acid fragments, which can be used for example as hybridization probes or primers for identifying or amplifying coding nucleic acids according to the invention.

The present invention also relates to nucleic acids with a certain degree of “identity” to the sequences specifically disclosed herein. “Identity” between two nucleic acids means identity of the nucleotides, in each case over the entire length of the nucleic acid.

The “identity” between two nucleotide sequences (the same applies to peptide or amino acid sequences) is a function of the number of nucleotide residues (or amino acid residues) or that are identical in the two sequences when an alignment of these two sequences has been generated. Identical residues are defined as residues that are the same in the two sequences in a given position of the alignment. The percentage of sequence identity, as used herein, is calculated from the optimal alignment by taking the number of residues identical between two sequences dividing it by the total number of residues in the shortest sequence and multiplying by 100. The optimal alignment is the alignment in which the percentage of identity is the highest possible. Gaps may be introduced into one or both sequences in one or more positions of the alignment to obtain the optimal alignment. These gaps are then taken into account as non-identical residues for the calculation of the percentage of sequence identity. Alignment for the purpose of determining the percentage of amino acid or nucleic acid sequence identity can be achieved in various ways using computer programs and for instance publicly available computer programs available on the world wide web.

Particularly, the BLAST program (Tatiana et al, FEMS Microbiol Lett., 1999, 174:247-250, 1999) set to the default parameters, available from the National Center for Biotechnology Information (NCBI) website at ncbi.nlm.nih.gov/BLAST/bl2seq/wblast2.cgi, can be used to obtain an optimal alignment of protein or nucleic acid sequences and to calculate the percentage of sequence identity.

In another example the identity may be calculated by means of the Vector NTI Suite 7.1 program of the company Informax (USA) employing the Clustal Method (Higgins D G, Sharp P M. ((1989))) with the following settings:

Multiple alignment parameters:

| | |
|---|---|
| Gap opening penalty | 10 |
| Gap extension penalty | 10 |
| Gap separation penalty range | 8 |
| Gap separation penalty | off |
| % identity for alignment delay | 40 |
| Residue specific gaps | off |
| Hydrophilic residue gap | off |
| Transition weighing | 0 |
| | 40 |

Pairwise alignment parameter:

| FAST algorithm | on |
|---|---|
| K-tuple size | 1 |
| Gap penalty | 3 |
| Window size | 5 |
| Number of best diagonals | 5 |

Alternatively the identity may be determined according to Chenna, et al. (2003), the web page: http://www.ebi.ac.uk/Tools/clustalw/index.html # and the following settings

| | |
|---|---|
| DNA Gap Open Penalty | 15.0 |
| DNA Gap Extension Penalty | 6.66 |
| DNA Matrix | Identity |
| Protein Gap Open Penalty | 10.0 |
| Protein Gap Extension Penalty | 0.2 |
| Protein matrix | Gonnet |
| Protein/DNA ENDGAP | −1 |
| Protein/DNA GAPDIST | 4 |

All the nucleic acid sequences mentioned herein (single-stranded and double-stranded DNA and RNA sequences, for example cDNA and mRNA) can be produced in a known way by chemical synthesis from the nucleotide building blocks, e.g. by fragment condensation of individual overlapping, complementary nucleic acid building blocks of the double helix. Chemical synthesis of oligonucleotides can, for example, be performed in a known way, by the phosphoamidite method (Voet, Voet, 2nd edition, Wiley Press, New York, pages 896-897). The accumulation of synthetic oligonucleotides and filling of gaps by means of the Klenow fragment of DNA polymerase and ligation reactions as well as general cloning techniques are described in Sambrook et al. (1989), see below.

The nucleic acid molecules according to the invention can in addition contain non-translated sequences from the 3′ and/or 5′ end of the coding genetic region.

The invention further relates to the nucleic acid molecules that are complementary to the concretely described nucleotide sequences or a segment thereof.

The nucleotide sequences according to the invention make possible the production of probes and primers that can be used for the identification and/or cloning of homologous sequences in other cellular types and organisms. Such probes or primers generally comprise a nucleotide sequence region which hybridizes under “stringent” conditions (as defined herein elsewhere) on at least about 12, particularly at least about 25, for example about 40, 50 or 75 successive nucleotides of a sense strand of a nucleic acid sequence according to the invention or of a corresponding antisense strand.

“Homologous” sequences include orthologous or paralogous sequences. Methods of identifying orthologs or paralogs including phylogenetic methods, sequence similarity and hybridization methods are known in the art and are described herein.

“Paralogs” result from gene duplication that gives rise to two or more genes with similar sequences and similar functions. Paralogs typically cluster together and are formed by duplications of genes within related plant species. Paralogs are found in groups of similar genes using pair-wise Blast analysis or during phylogenetic analysis of gene families using programs such as CLUSTAL. In paralogs, consensus sequences can be identified characteristic to sequences within related genes and having similar functions of the genes.

“Orthologs”, or orthologous sequences, are sequences similar to each other because they are found in species that descended from a common ancestor. For instance, plant species that have common ancestors are known to contain many enzymes that have similar sequences and functions. The skilled artisan can identify orthologous sequences and predict the functions of the orthologs, for example, by constructing a polygenic tree for a gene family of one species using CLUSTAL or BLAST programs. A method for identifying or confirming similar functions among homologous sequences is by comparing of the transcript profiles in host cells or organisms, such as plants or microorganisms, overexpressing or lacking (in knockouts/knockdowns) related polypeptides. The skilled person will understand that genes having similar transcript profiles, with greater than 50% regulated transcripts in common, or with greater than 70% regulated transcripts in common, or greater than 90% regulated transcripts in common will have similar functions. Homologs, paralogs, orthologs and any other variants of the sequences herein are expected to function in a similar manner by making the host cells, organism such as plants or microorganisms producing enzymes of the invention.

The term “selectable marker” refers to any gene which upon expression may be used to select a cell or cells that include the selectable marker. Examples of selectable markers are described below. The skilled artisan will know that different antibiotic, fungicide, auxotrophic or herbicide selectable markers are applicable to different target species.

A nucleic acid molecule according to the invention can be recovered by means of standard techniques of molecular biology and the sequence information supplied according to the invention. For example, cDNA can be isolated from a suitable cDNA library, using one of the concretely disclosed complete sequences or a segment thereof as hybridization probe and standard hybridization techniques (as described for example in Sambrook, (1989)).

In addition, a nucleic acid molecule, comprising one of the disclosed sequences or a segment thereof, can be isolated by the polymerase chain reaction, using the oligonucleotide primers that were constructed on the basis of this sequence. The nucleic acid amplified in this way can be cloned in a suitable vector and can be characterized by DNA sequencing. The oligonucleotides according to the invention can also be produced by standard methods of synthesis, e.g. using an automatic DNA synthesizer.

Nucleic acid sequences according to the invention or derivatives thereof, homologues or parts of these sequences, can for example be isolated by usual hybridization techniques or the PCR technique from other bacteria, e.g. via genomic or cDNA libraries. These DNA sequences hybridize in standard conditions with the sequences according to the invention.

“Hybridize” means the ability of a polynucleotide or oligonucleotide to bind to an almost complementary sequence in standard conditions, whereas nonspecific binding does not occur between non-complementary partners in these conditions. For this, the sequences can be 90-100% complementary. The property of complementary sequences of being able to bind specifically to one another is utilized for example in Northern Blotting or Southern Blotting or in primer binding in PCR or RT-PCR.

Short oligonucleotides of the conserved regions are used advantageously for hybridization. However, it is also possible to use longer fragments of the nucleic acids according to the invention or the complete sequences for the hybridization. These “standard conditions” vary depending on the nucleic acid used (oligonucleotide, longer fragment or complete sequence) or depending on which type of nucleic acid—DNA or RNA—is used for hybridization. For example, the melting temperatures for DNA: DNA hybrids are approx. 10° C. lower than those of DNA:RNA hybrids of the same length.

For example, depending on the particular nucleic acid, standard conditions mean temperatures between 42 and 58° C. in an aqueous buffer solution with a concentration between 0.1 to 5×SSC (1×SSC=0.15 M NaCl, 15 mM sodium citrate, pH 7.2) or additionally in the presence of 50% formamide, for example 42° C. in 5×SSC. 50% formamide. Advantageously, the hybridization conditions for DNA:DNA hybrids are 0.1×SSC and temperatures between about 20° C. to 45° C., particularly between about 30° C. to 45° C. For DNA:RNA hybrids the hybridization conditions are advantageously 0.1×SSC and temperatures between about 30° C. to 55° C., particularly between about 45° C. to 55° C. These stated temperatures for hybridization are examples of calculated melting temperature values for a nucleic acid with a length of approx. 100 nucleotides and a G+C content of 50% in the absence of formamide. The experimental conditions for DNA hybridization are described in relevant genetics textbooks, for example Sambrook et al., 1989, and can be calculated using formulae that are known by a person skilled in the art, for example depending on the length of the nucleic acids, the type of hybrids or the G+C content. A person skilled in the art can obtain further information on hybridization from the following textbooks: Ausubel et al. (eds). (1985). Brown (ed) (1991).

“Hybridization” can in particular be carried out under stringent conditions. Such hybridization conditions are for example described in Sambrook (1989), or in Current Protocols in Molecular Biology. John Wiley & Sons. N.Y. (1989). 6.3.1-6.3.6.

As used herein, the term hybridization or hybridizes under certain conditions is intended to describe conditions for hybridization and washes under which nucleotide sequences that are significantly identical or homologous to each other remain bound to each other. The conditions may be such that sequences, which are at least about 70%, such as at least about 80%, and such as at least about 85%, 90%, or 95% identical, remain bound to each other. Definitions of low stringency, moderate, and high stringency hybridization conditions are provided herein.

Appropriate hybridization conditions can be selected by those skilled in the art with minimal experimentation as exemplified in Ausubel et al. (1995, Current Protocols in Molecular Biology, John Wiley & Sons, sections 2, 4, and 6). Additionally, stringency conditions are described in Sambrook et al. (1989, Molecular Cloning: A Laboratory Manual, 2nd ed., Cold Spring Harbor Press, chapters 7, 9, and 11).

As used herein, defined conditions of low stringency are as follows. Filters containing DNA are pretreated for 6 h at 40° C. in a solution containing 35% formamide. 5×SSC, 50 mM Tris-HCl (pH 7.5), 5 mM EDTA, 0.1% PVP, 0.1% Ficoll, 1% BSA, and 500 μg/ml denatured salmon sperm DNA. Hybridizations are carried out in the same solution with the following modifications: 0.02% PVP, 0.02% Ficoll, 0.2% BSA, 100 μg/ml salmon sperm DNA. 10% (wt/vol) dextran sulfate, and 5-20×106 32P-labeled probe is used. Filters are incubated in hybridization mixture for 18-20 h at 40° C. and then washed for 1.5 h at 55° C. In a solution containing 2×SSC, 25 mM Tris-HCl (pH 7.4), 5 mM EDTA, and 0.1% SDS. The wash solution is replaced with fresh solution and incubated an additional 1.5 h at 60° C. Filters are blotted dry and exposed for autoradiography.

As used herein, defined conditions of moderate stringency are as follows. Filters containing DNA are pretreated for 7 h at 50° C. in a solution containing 35% formamide, 5×SSC, 50 mM Tris-HCl (pH 7.5), 5 mM EDTA, 0.1% PVP. 0.1% Ficoll, 1% BSA, and 500 μg/ml denatured salmon sperm DNA. Hybridizations are carried out in the same solution with the following modifications: 0.02% PVP, 0.02% Ficoll, 0.2% BSA, 100 μg/ml salmon sperm DNA, 10% (wt/vol) dextran sulfate, and 5-20×106 32P-labeled probe is used. Filters are incubated in hybridization mixture for 30 h at 50° C., and then washed for 1.5 h at 55° C. In a solution containing 2×SSC, 25 mM Tris-HCl (pH 7.4), 5 mM EDTA, and 0.1% SDS. The wash solution is replaced with fresh solution and incubated an additional 1.5 h at 60° C. Filters are blotted dry and exposed for autoradiography.

As used herein, defined conditions of high stringency are as follows. Prehybridization of filters containing DNA is carried out for 8 h to overnight at 65° C. in buffer composed of 6×SSC, 50 mM Tris-HCl (pH 7.5), 1 mM EDTA, 0.02% PVP, 0.02% Ficoll, 0.02% BSA, and 500 μg/ml denatured salmon sperm DNA. Filters are hybridized for 48 h at 65° C. in the prehybridization mixture containing 100 μg/ml denatured salmon sperm DNA and 5-20×106 cpm of 32P-labeled probe. Washing of filters is done at 37° C. for 1 h in a solution containing 2×SSC, 0.01% PVP, 0.01% Ficoll, and 0.01% BSA. This is followed by a wash in 0.1×SSC at 50° C. for 45 minutes.

Other conditions of low, moderate, and high stringency well known in the art (e.g., as employed for cross-species hybridizations) may be used if the above conditions are inappropriate (e.g., as employed for cross-species hybridizations).

A detection kit for nucleic acid sequences encoding a polypeptide of the invention may include primers and/or probes specific for nucleic acid sequences encoding the polypeptide, and an associated protocol to use the primers and/or probes to detect nucleic acid sequences encoding the polypeptide in a sample. Such detection kits may be used to determine whether a plant, organism, microorganism or cell has been modified, i.e., transformed with a sequence encoding the polypeptide.

To test a function of variant DNA sequences according to an embodiment herein, the sequence of interest is operably linked to a selectable or screenable marker gene and expression of said reporter gene is tested in transient expression assays, for example, with microorganisms or with protoplasts or in stably transformed plants.

The invention also relates to derivatives of the concretely disclosed or derivable nucleic acid sequences.

Thus, further nucleic acid sequences according to the invention can be derived from the sequences specifically disclosed herein and can differ from it by one or more, like 1 to 20, in particular 1 to 15 or 5 to 10 additions, substitutions, insertions or deletions of one or several (like for example 1 to 10) nucleotides, and furthermore code for polypeptides with the desired profile of properties.

The invention also encompasses nucleic acid sequences that comprise so-called silent mutations or have been altered, in comparison with a concretely stated sequence, according to the codon usage of a special original or host organism.

According to a particular embodiment of the invention variant nucleic acids may be prepared in order to adapt its nucleotide sequence to a specific expression system. For example, bacterial expression systems are known to more efficiently express polypeptides if amino acids are encoded by particular codons. Due to the degeneracy of the genetic code, more than one codon may encode the same amino acid sequence, multiple nucleic acid sequences can code for the same protein or polypeptide, all these DNA sequences being encompassed by an embodiment herein. Where appropriate, the nucleic acid sequences encoding the polypeptides described herein may be optimized for increased expression in the host cell. For example, nucleic acids of an embodiment herein may be synthesized using codons particular to a host for improved expression.

The invention also encompasses naturally occurring variants, e.g. splicing variants or allelic variants, of the sequences described therein.

Allelic variants may have at least 60% homology at the level of the derived amino acid, particularly at least 80% homology, quite especially particularly at least 90% homology over the entire sequence range (regarding homology at the amino acid level, reference should be made to the details given above for the polypeptides). Advantageously, the homologies can be higher over partial regions of the sequences.

The invention also relates to sequences that can be obtained by conservative nucleotide substitutions (i.e. as a result thereof the amino acid in question is replaced by an amino acid of the same charge, size, polarity and/or solubility).

The invention also relates to the molecules derived from the concretely disclosed nucleic acids by sequence polymorphisms. Such genetic polymorphisms may exist in cells from different populations or within a population due to natural allelic variation. Allelic variants may also include functional equivalents. These natural variations usually produce a variance of 1 to 5% in the nucleotide sequence of a gene. Said polymorphisms may lead to changes in the amino acid sequence of the polypeptides disclosed herein. Allelic variants may also include functional equivalents.

Furthermore, derivatives are also to be understood to be homologs of the nucleic acid sequences according to the invention, for example animal, plant, fungal or bacterial homologs, shortened sequences, single-stranded DNA or RNA of the coding and noncoding DNA sequence. For example, homologs have, at the DNA level, a homology of at least 40%, particularly of at least 60%, especially particularly of at least 70%, quite especially particularly of at least 80% over the entire DNA region given in a sequence specifically disclosed herein.

Moreover, derivatives are to be understood to be, for example, fusions with promoters. The promoters that are added to the stated nucleotide sequences can be modified by at least one nucleotide exchange, at least one insertion, inversion and/or deletion, though without impairing the functionality or efficacy of the promoters. Moreover, the efficacy of the promoters can be increased by altering their sequence or can be exchanged completely with more effective promoters even of organisms of a different genus.

### 2.2 Constructs for Expressing Polypeptides of the Invention

In this context the following definitions apply:

“Expression of a gene” encompasses “heterologous expression” and “over-expression” and involves transcription of the gene and translation of the mRNA into a protein. Overexpression refers to the production of the gene product as measured by levels of mRNA, polypeptide and/or enzyme activity in transgenic cells or organisms that exceeds levels of production in non-transformed cells or organisms of a similar genetic background.

“Expression vector” as used herein means a nucleic acid molecule engineered using molecular biology methods and recombinant DNA technology for delivery of foreign or exogenous DNA into a host cell. The expression vector typically includes sequences required for proper transcription of the nucleotide sequence. The coding region usually codes for a protein of interest but may also code for an RNA, e.g., an antisense RNA, siRNA and the like.

An “expression vector” as used herein includes any linear or circular recombinant vector including but not limited to viral vectors, bacteriophages and plasmids. The skilled person is capable of selecting a suitable vector according to the expression system. In one embodiment, the expression vector includes the nucleic acid of an embodiment herein operably linked to at least one “regulatory sequence”, which controls transcription, translation, initiation and termination, such as a transcriptional promoter, operator or enhancer, or an mRNA ribosomal binding site and, optionally, including at least one selection marker. Nucleotide sequences are “operably linked” when the regulatory sequence functionally relates to the nucleic acid of an embodiment herein.

An “expression system” as used herein encompasses any combination of nucleic acid molecules required for the expression of one, or the co-expression of two or more polypeptides either in vivo of a given expression host, or in vitro. The respective coding sequences may either be located on a single nucleic acid molecule or vector, as for example a vector containing multiple cloning sites, or on a polycistronic nucleic acid, or may be distributed over two or more physically distinct vectors. As a particular example there may be mentioned an operon comprising a promotor sequence, one or more operator sequences and one or more structural genes each encoding an enzyme as described herein

As used herein, the terms “amplifying” and “amplification” refer to the use of any suitable amplification methodology for generating or detecting recombinant of naturally expressed nucleic acid, as described in detail, below. For example, the invention provides methods and reagents (e.g., specific degenerate oligonucleotide primer pairs, oligo dT primer) for amplifying (e.g., by polymerase chain reaction, PCR) naturally expressed (e.g., genomic DNA or mRNA) or recombinant (e.g., cDNA) nucleic acids of the invention in vivo, ex vivo or in vitro.

“Regulatory sequence” refers to a nucleic acid sequence that determines expression level of the nucleic acid sequences of an embodiment herein and is capable of regulating the rate of transcription of the nucleic acid sequence operably linked to the regulatory sequence. Regulatory sequences comprise promoters, enhancers, transcription factors, promoter elements and the like.

A “promoter”, a “nucleic acid with promoter activity” or a “promoter sequence” is understood as meaning, in accordance with the invention, a nucleic acid which, when functionally linked to a nucleic acid to be transcribed, regulates the transcription of said nucleic acid.

“Promoter” in particular refers to a nucleic acid sequence that controls the expression of a coding sequence by providing a binding site for RNA polymerase and other factors required for proper transcription including without limitation transcription factor binding sites, repressor and activator protein binding sites. The meaning of the term promoter also includes the term “promoter regulatory sequence”. Promoter regulatory sequences may include upstream and downstream elements that may influences transcription. RNA processing or stability of the associated coding nucleic acid sequence. Promoters include naturally-derived and synthetic sequences. The coding nucleic acid sequences is usually located downstream of the promoter with respect to the direction of the transcription starting at the transcription initiation site.

In this context, a “functional” or “operative” linkage is understood as meaning for example the sequential arrangement of one of the nucleic acids with a regulatory sequence. For example the sequence with promoter activity and of a nucleic acid sequence to be transcribed and optionally further regulatory elements, for example nucleic acid sequences which ensure the transcription of nucleic acids, and for example a terminator, are linked in such a way that each of the regulatory elements can perform its function upon transcription of the nucleic acid sequence. This does not necessarily require a direct linkage in the chemical sense. Genetic control sequences, for example enhancer sequences, can even exert their function on the target sequence from more remote positions or even from other DNA molecules. Preferred arrangements are those in which the nucleic acid sequence to be transcribed is positioned behind (i.e. at the 3′-end of) the promoter sequence so that the two sequences are joined together covalently. The distance between the promoter sequence and the nucleic acid sequence to be expressed recombinantly can be smaller than 200 base pairs, or smaller than 100 base pairs or smaller than 50 base pairs.

In addition to promoters and terminator, the following may be mentioned as examples of other regulatory elements: targeting sequences, enhancers, polyadenylation signals, selectable markers, amplification signals, replication origins and the like. Suitable regulatory sequences are described, for example, in Goeddel. Gene Expression Technology: Methods in Enzymology 185. Academic Press. San Diego, CA (1990).

The term “constitutive promoter” refers to an unregulated promoter that allows for continual transcription of the nucleic acid sequence it is operably linked to.

As used herein, the term “operably linked” refers to a linkage of polynucleotide elements in a functional relationship. A nucleic acid is “operably linked” when it is placed into a functional relationship with another nucleic acid sequence. For instance, a promoter, or rather a transcription regulatory sequence, is operably linked to a coding sequence if it affects the transcription of the coding sequence. Operably linked means that the DNA sequences being linked are typically contiguous. The nucleotide sequence associated with the promoter sequence may be of homologous or heterologous origin with respect to the plant to be transformed. The sequence also may be entirely or partially synthetic. Regardless of the origin, the nucleic acid sequence associated with the promoter sequence will be expressed or silenced in accordance with promoter properties to which it is linked after binding to the polypeptide of an embodiment herein. The associated nucleic acid may code for a protein that is desired to be expressed or suppressed throughout the organism at all times or, alternatively, at a specific time or in specific tissues, cells, or cell compartment. Such nucleotide sequences particularly encode proteins conferring desirable phenotypic traits to the host cells or organism altered or transformed therewith. More particularly, the associated nucleotide sequence leads to the production of the product or products of interest as herein defined in the cell or organism. Particularly, the nucleotide sequence encodes a polypeptide having an enzyme activity as herein defined.

The nucleotide sequence as described herein above may be part of an “expression cassette”. The terms “expression cassette” and “expression construct” are used synonymously. The (particularly recombinant) expression construct contains a nucleotide sequence which encodes a polypeptide according to the invention and which is under genetic control of regulatory nucleic acid sequences.

In a process applied according to the invention, the expression cassette may be part of an “expression vector”, in particular of a recombinant expression vector.

An “expression unit” is understood as meaning, in accordance with the invention, a nucleic acid with expression activity, which comprises a promoter as defined herein and, after functional linkage with a nucleic acid to be expressed, or a gene, regulates the expression, i.e. the transcription and the translation of said nucleic acid or said gene. It is therefore in this connection also referred to as a “regulatory nucleic acid sequence”. In addition to the promoter, other regulatory elements, for example enhancers, can also be present.

An “expression cassette” or “expression construct” is understood as meaning, in accordance with the invention, an expression unit which is functionally linked to the nucleic acid to be expressed or the gene to be expressed. In contrast to an expression unit, an expression cassette therefore comprises not only nucleic acid sequences which regulate transcription and translation, but also the nucleic acid sequences that are to be expressed as protein as a result of transcription and translation.

The terms “expression” or “overexpression” describe, in the context of the invention, the production or increase in intracellular activity of one or more polypeptides in a microorganism, which are encoded by the corresponding DNA. To this end, it is possible for example to introduce a gene into an organism, replace an existing gene with another gene, increase the copy number of the gene(s), use a strong promoter or use a gene which encodes for a corresponding polypeptide with a high activity; optionally, these measures can be combined.

Particularly such constructs according to the invention comprise a promoter 5′-upstream of the respective coding sequence and a terminator sequence 3′-downstream and optionally other usual regulatory elements, in each case in operative linkage with the coding sequence.

Nucleic acid constructs according to the invention comprise in particular a sequence coding for a polypeptide for example derived from the amino acid related SEQ ID NOs as described therein or the reverse complement thereof, or derivatives and homologs thereof and which have been linked operatively or functionally with one or more regulatory signals, advantageously for controlling, for example increasing, gene expression.

In addition to these regulatory sequences, the natural regulation of these sequences may still be present before the actual structural genes and optionally may have been genetically modified so that the natural regulation has been switched off and expression of the genes has been enhanced. The nucleic acid construct may, however, also be of simpler construction, i.e. no additional regulatory signals have been inserted before the coding sequence and the natural promoter, with its regulation, has not been removed. Instead, the natural regulatory sequence is mutated such that regulation no longer takes place and the gene expression is increased.

A preferred nucleic acid construct advantageously also comprises one or more of the already mentioned “enhancer” sequences in functional linkage with the promoter, which sequences make possible an enhanced expression of the nucleic acid sequence. Additional advantageous sequences may also be inserted at the 3′-end of the DNA sequences, such as further regulatory elements or terminators. One or more copies of the nucleic acids according to the invention may be present in a construct. In the construct, other markers, such as genes which complement auxotrophisms or antibiotic resistances, may also optionally be present so as to select for the construct.

Examples of suitable regulatory sequences are present in promoters such as cos, tac, trp, tet, trp-tet, lpp, lac, lpp-lac, lacI^{q}, T7, T5, T3, gal, trc, ara, rhaP (rhaPBAD) SP6, lambda-P_{R} or in the lambda-P_{L} promoter, and these are advantageously employed in Gram-negative bacteria. Further advantageous regulatory sequences are present for example in the Gram-positive promoters amy and SPO2, in the yeast or fungal promoters ADC1, MFalpha, AC, P-60, CYC1, GAPDH, TEF, rp28. ADH. Artificial promoters may also be used for regulation.

For expression in a host organism, the nucleic acid construct is inserted advantageously into a vector such as, for example, a plasmid or a phage, which makes possible optimal expression of the genes in the host. Vectors are also understood as meaning, in addition to plasmids and phages, all the other vectors which are known to the skilled worker, that is to say for example viruses such as SV40, CMV, baculovirus and adenovirus, transposons, IS elements, phasmids, cosmids and linear or circular DNA or artificial chromosomes. These vectors are capable of replicating autonomously in the host organism or else chromosomally. These vectors are a further development of the invention. Binary or cpo-integration vectors are also applicable.

Suitable plasmids are, for example, in E. coli pLG338, pACYC184, pBR322, pUC18, pUC19, pKC30, pRep4, pHS1, pKK223-3, pDHE19.2, pHS2, pPLc236, pMBL24, pLG200, pUR290, pIN-III¹¹³-BI, λgt11 or pBdCI, in Streptomyces pIJ101, pIJ364, pIJ702 or pIJ361, in Bacillus pUB110, pC194 or pBD214, in Corynebacterium pSA77 or pAJ667, in fungi pALS1, pIL2 or pBB116, in yeasts 2alphaM, pAG-1, YEp6, YEp13 or pEMBLYe23 or in plants pLGV23, pGHlac⁺, pBIN19, pAK2004 or pDH51. The abovementioned plasmids are a small selection of the plasmids which are possible. Further plasmids are well known to the skilled worker and can be found for example in the book Cloning Vectors (Eds. Pouwels P. H. et al. Elsevier, Amsterdam-New York-Oxford, 1985, ISBN 0 444 904018).

In a further development of the vector, the vector which comprises the nucleic acid construct according to the invention or the nucleic acid according to the invention can advantageously also be introduced into the microorganisms in the form of a linear DNA and integrated into the host organism's genome via heterologous or homologous recombination. This linear DNA can consist of a linearized vector such as a plasmid or only of the nucleic acid construct or the nucleic acid according to the invention.

For optimal expression of heterologous genes in organisms, it is advantageous to modify the nucleic acid sequences to match the specific “codon usage” used in the organism. The “codon usage” can be determined readily by computer evaluations of other, known genes of the organism in question.

An expression cassette according to the invention is generated by fusing a suitable promoter to a suitable coding nucleotide sequence and a terminator or polyadenylation signal. Customary recombination and cloning techniques are used for this purpose, as are described, for example, in T. Maniatis, E. F. Fritsch and J. Sambrook, Molecular Cloning: A Laboratory Manual. Cold Spring Harbor Laboratory, Cold Spring Harbor, NY (1989) and in T. J. Silhavy, M. L. Berman and L. W. Enquist, Experiments with Gene Fusions, Cold Spring Harbor Laboratory, Cold Spring Harbor, NY (1984) and in Ausubel, F. M. et al., Current Protocols in Molecular Biology, Greene Publishing Assoc. and Wiley Interscience (1987).

For expression in a suitable host organism, the recombinant nucleic acid construct or gene construct is advantageously inserted into a host-specific vector which makes possible optimal expression of the genes in the host. Vectors are well known to the skilled worker and can be found for example in “cloning vectors” (Pouwels P. H. et al., Ed., Elsevier, Amsterdam-New York-Oxford, 1985).

An alternative embodiment of an embodiment herein provides a method to “alter gene expression” in a host cell. For instance, the polynucleotide of an embodiment herein may be enhanced or overexpressed or induced in certain contexts (e.g. upon exposure to certain temperatures or culture conditions) in a host cell or host organism.

Alteration of expression of a polynucleotide provided herein may also result in ectopic expression which is a different expression pattern in an altered and in a control or wild-type organism. Alteration of expression occurs from interactions of polypeptide of an embodiment herein with exogenous or endogenous modulators, or as a result of chemical modification of the polypeptide. The term also refers to an altered expression pattern of the polynucleotide of an embodiment herein which is altered below the detection level or completely suppressed activity.

In one embodiment, provided herein is also an isolated, recombinant or synthetic polynucleotide encoding a polypeptide or variant polypeptide provided herein.

In one embodiment, several polypeptide encoding nucleic acid sequences are co-expressed in a single host, particularly under control of different promoters. In another embodiment, several polypeptide encoding nucleic acid sequences can be present on a single transformation vector or be co-transformed at the same time using separate vectors and selecting transformants comprising both chimeric genes. Similarly, one or polypeptide encoding genes may be expressed in a single plant, cell, microorganism or organism together with other chimeric genes.

### 3. Hosts to be Applied for the Present Invention

Depending on the context, the term “host” can mean the wild-type host or a genetically altered, recombinant host or both.

In principle, all prokaryotic or eukaryotic organisms may be considered as host or recombinant host organisms for the nucleic acids or the nucleic acid constructs according to the invention.

Using the vectors according to the invention, prokaryotic or eukaryotic recombinant hosts can be produced, which are for example transformed with at least one vector according to the invention and can be used for producing the polypeptides according to the invention. Advantageously, the recombinant constructs according to the invention, described above, are introduced into a suitable host system and expressed. Particularly common cloning and transfection methods, known by a person skilled in the art, are used, for example co-precipitation, protoplast fusion, electroporation, retroviral transfection and the like, for expressing the stated nucleic acids in the respective expression system. Suitable systems are described for example in Current Protocols in Molecular Biology, F. Ausubel et al., Ed., Wiley Interscience, New York 1997, or Sambrook et al. Molecular Cloning: A Laboratory Manual. 2nd edition, Cold Spring Harbor Laboratory, Cold Spring Harbor Laboratory Press, Cold Spring Harbor, NY, 1989.

For example, microorganisms such as bacteria are used as host organisms. For example, gram-positive or gram-negative bacteria are used, particularly bacteria of the families Enterobacteriaceae, Pseudomonadaceae, Rhizobiaceae, Streptomycetaceae, Streptococcaceae or Nocardiaceae, especially particularly bacteria of the genera Escherichia, Pseudomonas, Streptomyces, Lactococcus, Nocardia, Burkholderia, Salmonella, Agrobacterium, Clostridium or Rhodococcus. The genus and species Escherichia coli is quite especially preferred. Advantageously also yeasts of families like Saccharomyces or Pichia are suitable hosts.

Alternatively, eukaryotic cells may be used as hosts. Eukaryotic cells of the present invention can be selected from, but are not limited to, mammalian cells, insect cells, yeast cells and plant cells. The eukaryotic cells of the invention may be present as individual cells or may be part of a tissue (e.g. a cell in a (cultured) tissue, organ or entire organism).

Entire plants or plant cells may serve as natural or recombinant host. As non-limiting examples the following plants or cells derived therefrom may be mentioned the genera Nicotiana, in particular Nicotiana benthamiana and Nicotiana tabacum (tobacco); as well as Arabidopsis, in particular Arabidopsis thaliana.

Particular non-limiting examples of insect cells are Sf21, Sf9 and High Five cells. Particular non-limiting examples of mammalian cells that areHEK293, HEK293T, HEK293F, CHO, CHO-S, COS, and HeLa cells.

Depending on the host organism, the organisms used in the method according to the invention are grown or cultured in a manner known by a person skilled in the art. Culture can be batchwise, semi-batchwise or continuously. Nutrients can be present at the beginning of fermentation or can be supplied later, semicontinuously or continuously. This is also described in more detail below.

4. POIs Comprising One or More than One UNAA and their Preparation

### 4.1 POIS

A POI of the present invention, in general relates to any form of polypeptide or protein molecule, which may be recombinantly produced in any suitable host cell system or cell-free expression system as described above in the presence of at least one ncAA and a pyrrolysyl tRNA synthetase of the present invention and a tRNA^{Pyl} as described above.

In a particular embodiment a POI is utilized to form as a “targeting agent”.

The primary object of such targeting agent is the formation of a covalent or noncovalent linkage with a particular “target”. A secondary object of the targeting agent is the targeted transport of a “payload molecule” to said target. In order to achieve said second object the POI has to be combined (reversibly or irreversibly) with at least one payload molecule. For this purpose said POI has to be functionalised by introducing said at least one ncAA. The functionalized POI carrying said at least one ncAA may then be linked to said at least one payload molecule through bioconjugation via said ncAA residue. Said ncAA is reactive with a payload molecule which in turn carries a corresponding moiety reactive with said at least one ncAA residue of the POI. The thus obtained bioconjugate. i.e. the targeting agent, allows the transfer of the payload molecule to the intended target.

For example, a “target” can be any molecule, which is present in and/or on an organism, tissue or cell. Such targets may be nonspecific or specific for a particular organism, tissue or cell. Targets include cell surface targets, e.g. receptors, glycoproteins, glycans, carbohydrates; structural proteins. e.g. amyloid plaques: abundant extracellular targets such as in stroma, extracellular matrix targets such as growth factors, and proteases; intracellular targets, e.g. surfaces of Golgi bodies, surfaces of mitochondria, RNA, DNA, enzymes, components of cell signaling pathways; and/or foreign bodies, e.g. pathogens such as viruses, bacteria, fungi, yeast or parts thereof.

Examples of targets include compounds such as proteins of which the presence or expression level is correlated with a certain tissue or cell type or of which the expression level is up-regulated or down-regulated in a certain disorder.

In particular, such target is a protein such as a (internalizing or non-internalizing) receptor.

Targets can be selected from any suitable targets within the human or animal body or on a pathogen or parasite.

Non-limiting examples of suitable targets include but are not limited to a group comprising cellular components such as cell membranes and cell walls, receptors such as cell membrane receptors, intracellular structures such as Golgi bodies or mitochondria, enzymes, receptors, DNA, RNA, viruses or viral particles, macrophages, tumor-associated macrophages, antibodies, proteins, carbohydrates, monosaccharides, polysaccharides, cytokines, hormones, steroids, somatostatin receptor, monoamine oxidase, muscarinic receptors, myocardial sympatic nerve system, leukotriene receptors, e.g. on leukocytes, urokinase plasminogen activator receptor (uPAR), folate receptor, apoptosis marker, (anti-) angiogenesis marker, gastrin receptor, dopaminergic system, serotonergic system, GABAergic system, adrenergic system, cholinergic system, opioid receptors, GPIIb/IIIa receptor and other thrombus related receptors, fibrin, calcitonin receptor, tuftsin receptor, P-glycoprotein, neurotensin receptors, neuropeptide receptors, substance P receptors, NK receptor, CCK receptors, sigma receptors, interleukin receptors, herpes simplex virus tyrosine kinase, human tyrosine kinase, integrin receptor, fibronectin targets, AOC3, ALK, AXL, C242, CA-125, CCL11, CCR5, CD2, CD3, CD4, CD5, CD15, CA15-3, CD18, CD19, CA19-9, CD20, CD21, CD22, CD23, CD25, CD28, CD30, CD31, CD33, CD37, CD38, CD40, CD41, CD44v6, CD45, CD51, CD52, CD54, CD56, CD62E, CD62P, CD62L, CD70, CD72, CD74, CD79-B, CD80, CD105, CD125, CD138, CD141, CD147, CD152, CD154, CD174, CD227, CD326, CD340, VEGF/EGF and VEGF/EGF receptors, VEGF-A, VEGFR2, VEGFR1, TAG72, CEA, MUC1, MUC16, GPNMB, PSMA, Cripto, Tenascin C, Melanocortin-1 receptor, G250, HLA DR, ED-B, TMEFF2, EphB2, EphB4, EphA2, FAP, Mesothelin, GD2, GD3, CAIX, 5T4, clumping factor, CTLA-4, CXCR2, FGFR1, FGFR2, FGFR3, FGFR4, NaPi2b, NOTCH1, NOTCH2, NOTCH3, NOTCH4, ErbB2, ErbB3, EpCAM, FLT3, HGF, HER2, HER3, HMI24, ICAM, ICOS-L, IGF-1 receptor, TRPV1, CFTR, gdNMB, CA9, c-KIT, c-MET, ACE, APP, adrenergic receptor beta2, Claudine 3, RON, ROR1, PD-L1, PD-L2, B7-H3, B7-H4, IL-2 receptor, IL-4 receptor, IL-13 receptor, integrins, IFN-alpha, IFN-gamma, IgE, IGF-1 receptor, IL-1, IL-4, IL-5, IL-6, IL-12, IL-13, IL-22, IL-23, interferon receptor, ITGB2 (CD18), LFA-1 (CDl 1a), L-selectin, P-selectin, E-selectin, mucin, myostatin, NCA-90, NGF, PDGFR alpha, prostatic carcinoma cells, Pseudomonas aeruginosa, rabies, RANKL, respiratory syncytial virus, Rhesus factor, SLAMF7, sphingosine-1-phosphate, TGF-1, TGFbeta2, TGFbeta, TNFalpha, TRAIL-R1, TRAIL-R2, CTAA 16.88, vimentin, matrix metalloproteinases (MMP) such as MMP2, MMP9, MMP14, LDL receptor, endoglins, polysialic acids and their corresponding lectins. An example of fibronectin targets are the alternatively spliced extra-domain-A (ED-A) and extra-domain-B (ED-B) of fibronectin. Non-limiting examples of targets in stroma can be found in V. Hofmeister, D. Schrama, J. C. Becker, Cancer Immun.; Immunother. 2008, 57, 1, the contents of which are hereby incorporated by reference.

More particularly, in order to allow a (specific) targeting of the above-listed targets, the targeting agent can comprise compounds comprising an ncAA-functionalized peptide sequence. Such compounds include but are not limited to antibodies, antibody derivatives, antibody fragments, antibody (fragment) fusions (e.g. bi-specific and tri-specific mAb fragments or derivatives), proteins, peptides, e.g. octreotide and derivatives, VIP, MSH, LHRH, chemotactic peptides, bombesin, elastin, peptide mimetics, receptor agonists and antagonists, cytokines, hormones, steroids, toxins.

According to a particular aspect, the target is a receptor and a targeting agent is employed, which is capable of specific binding to the target. Suitable targeting agents include but are not limited to, the ligand of such a receptor or a part thereof, which still binds to the receptor, e.g. a receptor binding peptide in the case of receptor binding protein ligands.

Other examples of targeting agents of protein nature include insulin, transferrin, fibrinogen-gamma fragment, thrombospondin, claudin, apolipoprotein E, Affibody molecules such as for example ABY-025, Ankyrin repeat proteins, ankyrin-like repeat proteins, interferons, e.g. alpha, beta, and gamma interferon, interleukins, lymphokines, colony stimulating factors and protein growth factor, such as tumor growth factor, e.g. alpha, beta tumor growth factor, platelet-derived growth factor (PDGF), uPAR targeting protein, apolipoprotein, LDL, annexin V, endostatin, and angiostatin.

Examples of peptides molecules, like antibody, as used in targeting agents include LHRH receptor targeting peptides, EC-1 peptide, RGD peptides, HER2-targeting peptides, PSMA targeting peptides, somatostatin-targeting peptides, bombesin. Other examples of targeting agents include lipocalins, such as anticalins.

One particular embodiment uses Affibodies™ and multimers and derivatives.

In one particular embodiment antibodies are used to form a targeting agent. While antibodies or immunoglobulins derived from IgG antibodies are particularly well-suited for use in this invention, immunoglobulins from any of the classes or subclasses may be selected, e.g. IgG, IgA, IgM, IgD and IgE. Suitably, the immunoglobulin is of the class IgG including but not limited to IgG subclasses (IgG1, 2, 3 and 4) or the class IgM which is able to specifically bind to a specific epitope on an antigen. Antibodies can be intact immunoglobulins derived from natural sources or from recombinant sources and can be immunoreactive portions of intact immunoglobulins. Antibodies may exist in a variety of forms including, for example, polyclonal antibodies, monoclonal antibodies, camelized single domain antibodies, recombinant antibodies, anti-idiotype antibodies, multispecific antibodies, antibody fragments, such as, Fv, VHH, Fab, F(ab)2, Fab′, Fab′-SH, F(ab′)2, single chain variable fragment antibodies (scFv), tandem/bis-scFv, Fc, pFc′, scFv-Fc, disulfide Fv (dsFv), bispecific antibodies (bc-scFv) such as BiTE antibodies, trispecific antibody derivatives such as tribodies, camelid antibodies, minibodies, nanobodies, resurfaced antibodies, humanized antibodies, fully human antibodies, single domain antibodies (sdAb, also known as Nanobody™), chimeric antibodies, chimeric antibodies comprising at least one human constant region, dual-affinity antibodies such as dual-affinity retargeting proteins (DART™), and multimers and derivatives thereof, such as divalent or multivalent single-chain variable fragments (e.g. di-scFvs, tri-scFvs) including but not limited to minibodies, diabodies, triabodies, tribodies, tetrabodies, and the like, and multivalent antibodies. Reference is made to [Trends in Biotechnology 2015, 33, 2, 65], [Trends Biotechnol. 2012, 30, 575-582], and [Cane. Gen. Prot. 2013 10, 1-18], and [BioDrugs 2014, 28, 331-343], the contents of which are hereby incorporated by reference.

“Antibody fragment” refers to at least a portion of the variable region of the immunoglobulin that binds to its target, i.e. the antigen-binding region.

Other embodiments use antibody mimetics as targeting agents, such as but not limited to Affimers, Anticalins, Avimers, Alphabodies, Affibodies, DARPins, and multimers and derivatives thereof; reference is made to [Trends in Biotechnology 2015, 33, 2, 65], the contents of which is hereby incorporated by reference.

For the avoidance of doubt, in the context of this invention the term “antibody” is meant to encompass all of the antibody variations, fragments, derivatives, fusions, analogs and mimetics outlined in this paragraph, unless specified otherwise.

In a preferred embodiment the targeting agent is selected from agents derived from antibodies and antibody derivatives such as antibody fragments, fragment fusions, proteins, peptides, peptide mimetics.

In another preferred embodiment the targeting agent is selected from agents derived from antibody fragments, fragment fusions, and other antibody derivatives that do not contain a Fc domain.

Typical non-limiting examples of antibody molecules to be further modified to form ncAA modified POI of the present invention are selected form biologically, in particular pharmacologically active antibody molecules. Non-limiting examples are selected form the following group: trastuzumab, bevacizumab, cetuximab, panitumumab, ipilimumab, rituximab, alemtuzumab, ofatumumab, gemtuzumab, brentuximab, ibritumomab, tositumomab, pertuzumab, adecatumumab, IGN101, INA01 labetuzumab, hua33, pemtumomab, oregovomab, minretumomab (CC49), cG250, J591, MOv-18, farletuzumab (MORAb-003), 3F8, ch14,18, KW-2871, hu3S193, lgN31 1, IM-2C6, CDP-791, etaracizumab, volociximab, nimotuzumab, MM-121, AMG 102, METMAB, SCH 900105, AVE1642, IMC-A12, MK-0646, R1507, CP 751871 KB004, III A4, mapatumumab, HGS-ETR2, CS-1008, denosumab, sibrotuzumab, F19, 81 C6, pinatuzumab, lifastuzumab, glembatumumab, coltuximab, lorvotuzumab, indatuximab, anti-PSMA, MLN-0264, ABT-414, milatuzumab, ramucirumab, abagovomab, abituzumab, adecatumumab, afutuzumab, altumomab pentetate, amatuximab, anatumomab, anetumab, apolizumab, arcitumomab, ascrinvacumab, atezolizumab, bavituximab, bectumomab, belimumab, bivatuzumab, brontictuzumab, cantuzumab, capromab, catumaxomab, citatuzumab, cixutumumab, clivatuzumab, codrituzumab, conatumumab, dacetuzumab, dallotuzumab, daratumumab, demcizumab, denintuzumab, depatuxizumab, derlotuximab, detumomab, dinutuximab, drozitumab, duligotumab, durvalumab, dusigitumab, ecromeximab, edrecolomab, elgemtumab, emactuzumab, enavatuzumab emibetuzumab, enfortumab, enoblituzumab, ensituximab, epratuzumab, ertumaxomab, etaracizumab, farletuzumab, ficlatuzumab, figitumumab, flanvotumab, futuximab, galiximab, ganitumab, icrucumab, igovomab, imalumab, imgatuzumab, indusatumab, inebilizumab, intetumumab, iratumumab, isatuximab, lexatuzumab, lilotomab, lintuzumab, lirilumab, lucatumumab, lumretuzumab, margetuximab, matuzumab, mirvetuximab, mitumomab, mogamulizumab, moxetumomab, nacolomab, naptumomab, narnatumab, necitumumab, nesvacumab, nimotuzumab, nivolumab, nofetumomab, obinutuzumab, ocaratuzumab, ofatumumab, olaratumab, onartuzumab, ontuxizumab, oportuzumab, oregovomab, otlertuzumab, pankomab, parsatuzumab, pasotuxizumab, patritumab, pembrolizumab, pemtumomab, pidilizumab, pintumomab, polatuzumab, pritumumab, quilizumab, racotumomab, ramucirumab, rilotumumab, robatumumab, sacituzumab, samalizumab, satumomab, seribantumab, siltuximab, sofituzumab, tacatuzumab, taplitumomab, tarextumab, tenatumomab, teprotumumab, tetulomab, ticilimumab, tigatuzumab, tositumomab, tovetumab, tremelimumab, tucotuzumab, ublituximab, ulocuplumab, urelumab, utomilumab, vadastuximab, vandortuzumab, vantictumab, vanucizumab, varlilumab, veltuzumab, vesencumab, volociximab, vorsetuzumab votumumab, zalutumumab, zatuxima, combination and derivatives thereof, as well as other monoclonal antibodies targeting CAI 25, CAI 5-3, CAI 9-9, L6, Lewis Y, Lewis X, alpha fetoprotein, CA 242, placental alkaline phosphatase, prostate specific antigen, prostate specific membrane antigen, prostatic acid phosphatase, epidermal growth factor, MAGE-1, MAGE-2, MAGE-3, MAGE-4, transferrin receptor, p97, MUCI, CEA, gplOO, MARTI, IL-2 receptor, CD20, CD52, CD33, CD22, human chorionic gonadotropin, CD38, CD40, mucin, P21, MPG, and Neu oncogene product.

According to a further particular embodiment of the invention, the target and targeting agent are selected so as to result in the specific or increased targeting of a tissue or disease, such as cancer, an inflammation, an infection, a cardiovascular disease, e.g. thrombus, atherosclerotic lesion, hypoxic site, e.g. stroke, tumor, cardiovascular disorder, brain disorder, apoptosis, angiogenesis, an organ, and reporter gene/enzyme. This can be achieved by selecting targets with tissue-, cell- or disease-specific expression.

By way of example, the targeting agent specifically binds or complexes with a cell surface molecule, such as a cell surface receptor or antigen, for a given cell population. Following specific binding or complexing of the targeting agent with the receptor, the drug will enter the cell.

As used herein, a targeting agent that “specifically binds or complexes with” or “targets” a cell surface molecule, an extracellular matrix target, or another target, preferentially associates with the target via intermolecular forces. For example, the ligand can preferentially associate with the target with a dissociation constant (Kd or KD) of less than about 50 nM, less than about 5 nM, or less than about 500 pM.

### 4.2 POI Preparation

A POI comprising one or more than one UNAA residue can be prepared according to the present invention using a eukaryotic cell. The eukaryotic cell comprises (e.g., is fed with) at least one unnatural amino acid or a salt thereof corresponding to the UNAA residue(s) of the POI to be prepared. The eukaryotic cell further comprises:
(i) a PylRS of the invention and a tRNA^{Pyl}, wherein the PylRS is capable of (preferably selectively) acylating the tRNA^{Pyl} with the UNAA or salt thereof; and
(ii) a polynucleotide encoding the POI, wherein any position of the POI occupied by an UNAA residue is encoded by a codon (e.g. selector codon) that is the reverse complement of the anticodon of the tRNA^{Pyl}.

The eukaryotic cell is cultured so as to allow translation of the POI-encoding polynucleotide (ii), thereby producing the POI.

For producing a POI according to a method of the present invention, the translation in step (b) can be achieved by culturing the eukaryotic cell under suitable conditions, preferably in the presence of (e.g., in a culture medium containing) the UNAA or salt thereof, for a time suitable to allow translation at a ribosome of the cell. Depending on the polynucleotide(s) encoding the POI (and optionally the PylRS, tRNA^{Pyl}), it may be required to induce expression by adding a compound inducing transcription, such as, e.g., arabinose, isopropyl β-D-thiogalactoside (IPTG) or tetracycline. mRNA that encodes the POI (and comprises one or more than codon that is the reverse complement of the anticodon comprised by the tRNA^{Pyl}) is bound by the ribosome. Then, the polypeptide is formed by stepwise attachment of amino acids and UNAAs at positions encoded by codons which are recognized (bound) by respective aminoacyl tRNAs. Thus, the UNAA(s) is/are incorporated in the POI at the position(s) encoded by the codon(s) that is/are the reverse complement of the anticodon comprised by the tRNA^{Pyl}.

The eukaryotic cell may comprise a polynucleotide sequence encoding the PylRS of the invention which allows for expression of the PylRS by the cell. Likewise, the tRNA^{Pyl} may be produced by the eukaryotic cell based on a tRNA^{Pyl}-encoding polynucleotide sequence comprised by the cell. The PylRS-encoding polynucleotide sequence and the tRNA^{Pyl}-encoding polynucleotide sequence can be located either on the same polynucleotide or on separate polynucleotides.

Thus, in one embodiment, the present invention provides a method for producing a POI comprising one or more than one UNAA residue, wherein the method comprises the steps of:
(a) providing a eukaryotic cell comprising polynucleotide sequences encoding:
at least one PylRS of the invention,
at least one tRNA (tRNA^{Pyl}) that can be acylated by the PylRS, and
at least one POI, wherein any position of the POI occupied by an UNAA residue is encoded by a codon that is the reverse complement of the anticodon of the tRNA^{Pyl}; and
(b) allowing for translation of the polynucleotide sequences by the eukaryotic cell in the presence of an UNAA or a salt thereof, thereby producing the PylRS, tRNA^{Pyl} and the POI.

The eukaryotic cells used for preparing a POI comprising one or more than one unnatural amino acid residue as described herein can be prepared by introducing polynucleotide sequences encoding the PylRS, the tRNA^{Pyl} and the POI into a eukaryotic (host) cell. Said polynucleotide sequences can be located on the same polynucleotide or on separate polynucleotides, and can be introduced into the cell by methods known in the art (such as, e.g., using virus-mediated gene delivery, electroporation, microinjection, lipofection, or others).

After translation, the POI prepared according to the present invention may optionally be recovered and purified, either partially or substantially to homogeneity, according to procedures generally known in the art. Unless the POI is secreted into the culture medium, recovery usually requires cell disruption. Methods of cell disruption are well known in the art and include physical disruption, e.g., by (ultrasound) sonication, liquid-sheer disruption (e.g., via French press), mechanical methods (such as those utilizing blenders or grinders) or freeze-thaw cycling, as well as chemical lysis using agents which disrupt lipid-lipid, protein-protein and/or protein-lipid interactions (such as detergents), and combinations of physical disruption techniques and chemical lysis. Standard procedures for purifying polypeptides from cell lysates or culture media are also well known in the art and include, e.g., ammonium sulfate or ethanol precipitation, acid or base extraction, column chromatography, affinity column chromatography, anion or cation exchange chromatography, phosphocellulose chromatography, hydrophobic interaction chromatography, hydroxylapatite chromatography, lectin chromatography, gel electrophoresis and the like. Protein refolding steps can be used, as desired, in making correctly folded mature proteins. High performance liquid chromatography (HPLC), affinity chromatography or other suitable methods can be employed in final purification steps where high purity is desired. Antibodies made against the polypeptides of the invention can be used as purification reagents, i.e. for affinity-based purification of the polypeptides. A variety of purification/protein folding methods are well known in the art, including, e.g., those set forth in Scopes, Protein Purification, Springer, Berlin (1993); and Deutscher, Methods in Enzymology Vol. 182: Guide to Protein Purification, Academic Press (1990); and the references cited therein.

As noted, those of skill in the art will recognize that, after synthesis, expression and/or purification, polypeptides can possess a conformation different from the desired conformations of the relevant polypeptides. For example, polypeptides produced by prokaryotic systems often are optimized by exposure to chaotropic agents to achieve proper folding. During purification from, e.g., lysates derived from E. coli, the expressed polypeptide is optionally denatured and then renatured. This is accomplished, e.g., by solubilizing the proteins in a chaotropic agent such as guanidine HCl. In general, it is occasionally desirable to denature and reduce expressed polypeptides and then to cause the polypeptides to re-fold into the preferred conformation. For example, guanidine, urea, DTT, DTE, and/or a chaperonin can be added to a translation product of interest. Methods of reducing, denaturing and renaturing proteins are well known to those of skill in the art. Polypeptides can be refolded in a redox buffer containing, e.g., oxidized glutathione and L-arginine.

### 5. Payload Molecules

Payload molecules typically used may be selected from bioactive compounds, labeling agents, and chelators. Non-limiting examples thereof are given in the following sections.

### 5.1 Bioactive Compounds

Bioactive compounds include, but are not limited to, the following:

Bioactive compounds applicable according to the present invention include but are not limited to: small organic molecule drugs, steroids, lipids, proteins, aptamers, oligopeptides, oligonucleotides, oligosaccharides, as well as peptides, peptoids, amino acids, nucleotides, oligo- or polynucleotides, nucleosides, DNA, RNA, toxins, glycans and immunoglobulins.

Exemplary classes of bioactive compounds that can be used in the practice of the present invention include but are not limited to hormones, cytotoxins, antiproliferative/antitumor agents, antiviral agents, antibiotics, cytokines, anti-inflammatory agents, antihypertensive agents, chemosensitizing, photosensitizing and radiosensitizing agents, anti-AIDS substances, anti-viral agents, immunosuppressants, immunostimulants, enzyme inhibitors, anti-Parkinson agents, neurotoxins, channel blockers, modulators of cell-extracellular matrix interactions including cell growth inhibitors and anti-adhesion molecules, inhibitors of DNA, RNA or protein synthesis, steroidal and non-steriodal anti-inflammatory agents, anti-angiogenic factors, anti-Alzheimer agents.

In some embodiments, the bioactive compound is a low to medium molecular weight compound (e.g. about 200 to 5000 Da, about 200 to about 1500 Da, preferably about 300 to about 1000 Da).

Exemplary cytotoxic drugs are particularly those which are used for cancer therapy. Such drugs include, in general, DNA damaging agents, anti-metabolites, natural products and their analogs, enzyme inhibitors such as dihydro folate reductase inhibitors and thymidylate synthase inhibitors, DNA binders, DNA alkylators, radiation sensitizers, DNA intercalators, DNA cleavers, microtubule stabilizing and destabilizing agents, topoisomerases inhibitors. Examples include but are not limited to platinum-based drugs, the anthracycline family of drugs, the vinca drugs, the mitomycins, the bleomycins, the cytotoxic nucleosides, taxanes, lexitropsins, the pteridine family of drugs, diynenes, the podophyllotoxins, dolastatins, maytansinoids, differentiation inducers, and taxols. Particularly useful members of those classes include, for example, auristatins, maytansines, maytansinoids, calicheamicins, dactinomycines, duocarmycins, CC1065 and its analogs, camptothecin and its analogs, SN-38 and its analogs; DXd, tubulysin M, cryptophycins, pyrrolobenzodiazepines and pyrrolobenzodiazepine dimers (PBDs), pyridinobenzodiazepines (PDDs) and indolinobenzodiazepines (IBDs) (cf. US20210206763A1), methotrexate, methopterin, dichloromethotrexate, 5-fluorouracil, DNA minor groove binders, 6-mercaptopurine, cytosine arabinoside, melphalan, leurosine, leurosideine, actinomycin, anthracyclines (doxorubicin, epirubicin, idarubicin, daunorubicin, PNU-159682 (cf. U.S. Pat. No. 10,288,745 B2.) and its analogs, mitomycin C, mitomycin A, caminomycin, aminopterin, tallysomycin, podophyllotoxin and; podophyllotoxin derivatives such as etoposide or etoposide phosphate, vinblastine, vincristine, vindesine, taxol, taxotere retinoic acid, butyric acid, N8-acetyl spermidine, staurosporin, colchicine, camptothecin, esperamicin, ene-diynes, and their analogues, hemiasterlin and its analogues.

Other exemplary drug classes are angiogenesis inhibitors, cell cycle progression inhibitors, P13K/m-TOR/AKT pathway inhibitors, MAPK signaling pathway inhibitors, kinase inhibitors, protein chaperones inhibitors, HDAC inhibitors, PARP inhibitors, Wnt/Hedgehog signaling pathway inhibitors, RNA polymerase inhibitors, and protein degraders (cf. https://pubs.acs.org/doi/10.1021/acschembio.0c00285).

Examples of auristatins include dolastatin 10, monomethyl auristatin E (MMAE), auristatin F, monomethyl auristatin F (MMAF), auristatin F hydroxypropylamide (AF HPA), auristatin F phenylene diamine (AFP), monomethyl auristatin D (MMAD), auristatin PE, auristatin EB, auristatin EFP, auristatin TP and auristatin AQ. Suitable auristatins are also described in U.S. Publication Nos. 2003/0083263, 2011/0020343, and 2011/0070248; PCT Application; Publication Nos. WO09/117531, WO2005/081711, WO04/010957; WO02/088172 and WO01/24763, and U.S. Pat. Nos. 7,498,298; 6,884,869; 6,323,315; 6,239,104; 6,124,431; 6,034,065; 5,780,588; 5,767,237; 5,665,860; 5,663,149; 5,635,483; 5,599,902; 5,554,725; 5,530,097; 5,521,284; 5,504,191; 5,410,024; 5,138,036; 5,076,973; 4,986,988; 4,978,744; 4,879,278; 4,879,278; 4,816,444; and 4,486,414, the disclosures of which are incorporated herein by reference in their entirety.

Exemplary drugs include the dolastatins and analogues thereof including: dolastatin A (U.S. Pat. No. 4,486,414), dolastatin B (U.S. Pat. No. 4,486,414), dolastatin 10 (U.S. Pat. Nos. 4,486,444, 5,410,024, 5,504,191, 5,521,284, 5,530,097, 5,599,902, 5,635,483, 5,663,149, 5,665,860, 5,780,588, 6,034,065, 6,323,315), dolastatin 13 (U.S. Pat. No. 4,986,988), dolastatin 14 (U.S. Pat. No. 5,138,036), dolastatin 15 (U.S. Pat. No. 4,879,278), dolastatin 16 (U.S. Pat. No. 6,239,104), dolastatin 17 (U.S. Pat. No. 6,239,104), and dolastatin 18 (U.S. Pat. No. 6,239,104), each patent incorporated herein by reference in their entirety.

Exemplary maytansines, maytansinoids, such as DM-1 and DM-4, or maytansinoid analogs, including maytansinol and maytansinol analogs, are described in U.S. Pat. Nos. 4,424,219; 4,256,746; 4,294,757; 4,307,016; 4,313,946; 4,315,929; 4,331,598; 4,361,650; 4,362,663; 4,364,866; 4,450,254; 4,322,348; 4,371,533; 5,208,020; 5,416,064; 5,475,092; 5,585,499; 5,846,545; 6,333,410; 6,441,163; 6,716,821 and 7,276,497.

Other examples include mertansine and ansamitocin. Pyrrolobenzodiazepines (PBDs), which expressly include dimers and analogs, include but are not limited to those described in [Denny, Exp. Opin. Ther. Patents, 10(4):459-474 (2000)], [Hartley et al., Expert Opin Investig Drugs. 2011, 20(6):733-44], Antonow et al., Chem Rev. 2011, 111(4), 2815-64].

Calicheamicins include, e.g. enediynes, esperamicin, and those described in U.S. Pat. Nos. 5,714,586 and 5,739,116.

Examples of duocarmycins and analogs include CC1065, duocarmycin SA, duocarmycin A, duocarmycin B 1, duocarmycin B2, duocarmycin CI, duocarmycin C2, duocarmycin D, DU-86, KW-2189, adozelesin, bizelesin, carzelesin, seco-adozelesin. Other examples include those described in, for example, U.S. Pat. Nos. 5,070,092; 5,101,092; 5,187,186; 5,475,092; 5,595,499; 5,846,545; 6,534,660; 6,548,530; 6,586,618; 6,660,742; 6,756,397; 7,049,316; 7,553,816; 8,815,226; US20150104407; 61/988,011 filed May 2, 2014 and 62/010,972 filed Jun. 11, 2014; the disclosure of each of which is incorporated herein in its entirety.

Exemplary vinca alkaloids include vincristine, vinblastine, vindesine, and navelbine, and those disclosed in U.S. Publication Nos. 2002/0103136 and 2010/0305149, and in U.S. Pat. No. 7,303,749, the disclosures of which are incorporated herein by reference in their entirety.

Exemplary epothilone compounds include epothilone A, B, C, D, E, and F, and derivatives thereof. Suitable epothilone compounds and derivatives thereof are described, for example, in U.S. Pat. Nos. 6,956,036; 6,989,450; 6,121,029; 6,117,659; 6,096,757; 6,043,372; 5,969,145; and 5,886,026; and WO97/19086; WO98/08849; WO98/22461; WO98/25929; WO98/38192; WO99/01124; WO99/02514; WO99/03848; WO99/07692; WO99/27890; and WO99/28324; the disclosures of which are incorporated herein by reference in their entirety.

Exemplary cryptophycin compounds are described in U.S. Pat. Nos. 6,680,311 and; 6,747,021; the disclosures of which are incorporated herein by reference in their entirety.

Exemplary platinum compounds include cisplatin, carboplatin, oxaliplatin, iproplatin, ormaplatin, tetraplatin.

Exemplary DNA binding or alkylating drugs include CC-1065 and its analogs, anthracyclines, calicheamicins, dactinomycines, mitromycines, pyrrolobenzodiazepines, and the like.

Exemplary microtubule stabilizing and destabilizing agents include taxane compounds, such as paclitaxel, docetaxel, tesetaxel, and carbazitaxel; maytansinoids, auristatins and analogs thereof, vinca alkaloid derivatives, epothilones and cryptophycins.

Exemplary topoisomerase inhibitors include camptothecin and camptothecin derivatives, camptothecin analogs and non-natural camptothecins, such as, for example, CPT-11, SN-38,topotecan, 9-aminocamptothecin, rubitecan, gimatecan, karenitecin, silatecan, lurtotecan, exatecan, DXd, diflometotecan, belotecan, lurtotecan and S39625. Other camptothecin compounds that can be used in the present invention include those described in, for example, J. Med. Chem., 29:2358-2363 (1986); J. Med. Chem., 23:554 (1980); J. Med Chem., 30: 1774 (1987).

Angiogenesis inhibitors include, but are not limited to, MetAP2 inhibitors, VEGF inhibitors, PIGF inhibitors, VGFR inhibitors, PDGFR inhibitors, MetAP2 inhibitors. Exemplary VGFR and PDGFR inhibitors include sorafenib, sunitinib and vatalanib. Exemplary MetAP2 inhibitors include fumagillol analogs, meaning compounds that include the fumagillin core structure.

Exemplary cell cycle progression inhibitors include CDK inhibitors such as, for example, BMS-387032 and PD0332991; Rho-kinase inhibitors such as, for example, AZD7762; aurora kinase inhibitors such as, for example, AZD1152, MLN8054 and MLN8237; PLK inhibitors such as, for example, BI 2536, BI6727, GSK461364, ON-01910; and KSP inhibitors such as, for example, SB 743921, SB 715992, MK-0731, AZD8477, AZ3146 and ARRY-520.

Exemplary P13K/m-TOR/AKT signalling pathway inhibitors include phosphoinositide 3-kinase (P13K) inhibitors, GSK-3 inhibitors, ATM inhibitors, DNA-PK inhibitors and PDK-1 inhibitors.

Exemplary P13 kinases are disclosed in U.S. Pat. No. 6,608,053, and include BEZ235, BGT226, BKM120, CAL263, demethoxyviridin, GDC-0941, GSK615, IC87114, LY294002, Palomid 529, perifosine, PF-04691502, PX-866, SAR245408, SAR245409, SF1126, Wortmannin, XL147 and XL765.

Exemplary AKT inhibitors include, but are not limited to AT7867.

Exemplary MAPK signaling pathway inhibitors include MEK, Ras, JNK, B-Raf and p38 MAPK inhibitors.

Exemplary MEK inhibitors are disclosed in U.S. Pat. No. 7,517,944 and include GDC-; 0973, GSKI 120212, MSC1936369B, AS703026, R05126766 and R04987655, PD0325901, AZD6244, AZD8330 and GDC-0973.

Exemplary B-raf inhibitors include CDC-0879, PLX-4032, and SB590885.

Exemplary B p38 MAPK inhibitors include BIRB 796, LY2228820 and SB 202190. Exemplary receptor tyrosine kinases inhibitors include but are not limited to AEE788 (NVP-AEE 788), BIBW2992 (Afatinib), Lapatinib, Erlotinib (Tarceva), Gefitinib (Iressa), AP24534 (Ponatinib), ABT-869 (linifanib), AZD2171, CHR-258 (Dovitinib), Sunitinib (Sutent), Sorafenib (Nexavar), and Vatalinib.

Exemplary protein chaperon inhibitors include HSP90 inhibitors. Exemplary inhibitors include 17AAG derivatives, BIIB021, BIIB028, SNX-5422, NVP-AUY-922 and KW-2478.

Exemplary HDAC inhibitors include Belinostat (PXD101), CUDC-101, Droxinostat, ITF2357 (Givinostat, Gavinostat), JNJ-26481585, LAQ824 (NVP-LAQ824, Dacinostat), LBH-589 (Panobinostat), MC1568, MGCD0103 (Mocetinostat), MS-275 (Entinostat), PCI-24781, Pyroxamide (NSC 696085), SB939, Trichostatin A and Vorinostat (SAHA). Exemplary PARP inhibitors include iniparib (BSI 201), olaparib (AZD-2281), ABT-888 (Veliparib), AG014699, CEP9722, MK 4827, KU-0059436 (AZD2281), LT-673, 3-aminobenzamide, A-966492, and AZD2461.

Exemplary Wnt/Hedgehog signalling pathway inhibitors include vismodegib, cyclopamine and XAV-939.

Exemplary RNA polymerase inhibitors include amatoxins. Exemplary amatoxins include alpha-amanitins, beta amanitins, gamma amanitins, eta amanitins, amanullin, amanullic acid, amanisamide, amanon, and proamanullin.

Exemplary cytokines include IL-2, IL-7, IL-10, IL-12, IL-15, IL-21, TNF.

As non-limiting examples of particular drugs there may be mentioned Auristatins, Maytansinoids, PBDs, topoisomerase inhibitors, anthracyclines

In another embodiment, a combination of two or more different drugs as described above are used.

According to another embodiment, the bioactive compound may be selected from any synthetic or naturally occurring compounds comprising one or more natural and/or non-natural, proteinogenic and/or non-proteinogenic amino acid residues, such as in particular oligo- or polypeptides or proteins.

A particular group of such compounds comprises immunoglobulin molecules as for example antibodies, antibody derivatives, antibody fragments, antibody (fragment) fusions (e.g. bi-specific and tri-specific mAb fragments or derivatives), polyclonal or monoclonal antibodies, such as human, humanized, mouse or chimeric antibodies.

Typical non-limiting examples of antibodies for use in the present invention are selected form biologically, in particular pharmacologically active antibody molecules. Non-limiting examples are selected form the following group: trastuzumab, bevacizumab, cetuximab, panitumumab, ipilimumab, rituximab, alemtuzumab, ofatumumab, gemtuzumab, brentuximab, ibritumomab, tositumomab, pertuzumab, adecatumumab, IGN101, INA01 labetuzumab, hua33, pemtumomab, oregovomab, minretumomab (CC49), cG250, J591, MOv-18, farletuzumab (MORAb-003), 3F8, ch14,18, KW-2871, hu3S193, lgN31 1, IM-2C6, CDP-791, etaracizumab, volociximab, nimotuzumab, MM-121, AMG 102, METMAB, SCH 900105, AVE1642, IMC-A12, MK-0646, R1507, CP 751871, KB004, III A4, mapatumumab, HGS-ETR2, CS-1008, denosumab, sibrotuzumab, F19, 81 C6, pinatuzumab, lifastuzumab, glembatumumab, coltuximab, lorvotuzumab, indatuximab, anti-PSMA, MLN-0264, ABT-414, milatuzumab, ramucirumab, abagovomab, abituzumab, adecatumumab, afutuzumab, altumomab pentetate, amatuximab, anatumomab, anetumab, apolizumab, arcitumomab, ascrinvacumab, atezolizumab, bavituximab, bectumomab, belimumab, bivatuzumab, brontictuzumab, cantuzumab, capromab, catumaxomab, citatuzumab, cixutumumab, clivatuzumab, codrituzumab, conatumumab, dacetuzumab, dallotuzumab, daratumumab, demcizumab, denintuzumab, depatuxizumab, derlotuximab, detumomab, dinutuximab, drozitumab, duligotumab, durvalumab, dusigitumab, ecromeximab, edrecolomab, elgemtumab, emactuzumab, enavatuzumab emibetuzumab, enfortumab, enoblituzumab, ensituximab, epratuzumab, ertumaxomab, etaracizumab, farletuzumab, ficlatuzumab, figitumumab, flanvotumab, futuximab, galiximab, ganitumab, icrucumab, igovomab, imalumab, imgatuzumab, indusatumab, inebilizumab, intetumumab, iratumumab, isatuximab, lexatuzumab, lilotomab, lintuzumab, lirilumab, lucatumumab, lumretuzumab, margetuximab, matuzumab, mirvetuximab, mitumomab, mogamulizumab, moxetumomab, nacolomab, naptumomab, namatumab, necitumumab, nesvacumab, nimotuzumab, nivolumab, nofetumomab, obinutuzumab, ocaratuzumab, ofatumumab, olaratumab, onartuzumab, ontuxizumab, oportuzumab, oregovomab, otlertuzumab, pankomab, parsatuzumab, pasotuxizumab, patritumab, pembrolizumab, pemtumomab, pidilizumab, pintumomab, polatuzumab, pritumumab, quilizumab, racotumomab, ramucirumab, rilotumumab, robatumumab, sacituzumab, samalizumab, satumomab, seribantumab, siltuximab, sofituzumab, tacatuzumab, taplitumomab, tarextumab, tenatumomab, teprotumumab, tetulomab, ticilimumab, tigatuzumab, tositumomab, tovetumab, tremelimumab, tucotuzumab, ublituximab, ulocuplumab, urelumab, utomilumab, vadastuximab, vandortuzumab, vantictumab, vanucizumab, varlilumab, veltuzumab, vesencumab, volociximab, vorsetuzumab votumumab, zalutumumab, zatuxima, combination and derivatives thereof, as well as other monoclonal antibodies targeting CAI 25, CAI 5-3, CAI 9-9, L6, Lewis Y, Lewis X, alpha fetoprotein, CA 242, placental alkaline phosphatase, prostate specific antigen, prostate specific membrane antigen, prostatic acid phosphatase, epidermal growth factor, MAGE-1, MAGE-2, MAGE-3, MAGE-4, transferrin receptor, p97, MUCI, CEA, gplOO, MARTI, IL-2 receptor, CD20, CD52, CD33, CD22, human chorionic gonadotropin, CD38, CD40, mucin, P21, MPG, and Neu oncogene product.

### 5.2 Labelling Agents

Labeling agents which may be used according to the invention can comprise any type of label known in the art which does not inhibitor negatively affect reactivity of the tetrazine moiety.

Labels of the invention include, but are not limited to, dyes (e.g. fluorescent, luminescent, or phosphorescent dyes, such as dansyl, coumarin, fluorescein, acridine, rhodamine, silicon-rhodamine, BODIPY, or cyanine dyes), chromophores (e.g., phytochrome, phycobilin, bilirubin, etc.), radiolabels (e.g. radioactive forms of hydrogen, fluorine, carbon, phosphorous, sulphur, or iodine, such as tritium, fluorine-18, carbon-11, carbon-14, phosphorous-32, phosphorous-33, sulphur-33, sulphur-35, iodine-123, or iodine-125), MRI-sensitive spin labels, affinity tags (e.g. biotin, His-tag, Flag-tag, strep-tag, sugars, lipids, sterols, PEG-linkers, benzylguanines, benzylcytosines, or co-factors), polyethylene glycol groups (e.g., a branched PEG, a linear PEG, PEGs of different molecular weights, etc.), photocrosslinkers (such as p-azidoiodoacetanilide), NMR probes, X-ray probes, pH probes, IR probes, resins, solid supports.

In some embodiments, exemplary dyes can include an NIR contrast agent that fluoresces in the near infrared region of the spectrum. Exemplary near-infrared fluorophores can include dyes and other fluorophores with emission wavelengths (e.g., peak emission wavelengths) between about 630 and 1000 nm, e.g., between about 630 and 800 nm, between about 800 and 900 nm, between about 900 and 1000 nm, between about 680 and 750 nm, between about 750 and 800 nm, between about 800 and 850 nm, between about 850 and 900 nm, between about 900 and 950 nm, or between about 950 and 1000 nm. Fluorophores with emission wavelengths (e.g., peak emission wavelengths) greater than 1000 nm can also be used in the methods described herein.

In some embodiments, exemplary fluorophores include 7-amino-4-methylcoumarin-3-acetic acid (AMCA), TEXAS RED™ (Molecular Probes, Inc., Eugene, Oreg.), 5-(and -6)-carboxy-X-rhodamine, lissamine rhodamine B, 5-(and -6)-carboxyfluorescein, fluorescein-5-isothiocyanate (FITC), 7-diethylaminocoumarin-3-carboxylic acid, tetramethylrhodamine-5-(and-6)-isothiocyanate, 5-(and -6)-carboxytetramethylrhodamine, 7-hydroxycoumarin-3-carboxylic acid, 6-[fluorescein 5-(and -6)-carboxamido]hexanoic acid, N-(4,4-difluoro-5,7-dimethyl-4-bora-3a,4a diaza-3-indacenepropionic acid, eosin-5-isothiocyanate, erythrosin-5-isothiocyanate, and CASCADE™ blue acetylazide (Molecular Probes, Inc., Eugene, Oreg.) and ATTO dyes.

Further labelling agents are 177-Lutetium, 89-Zirkonium, 131-Iod, 68-Gallium, 99m-Technecium, 225-Actinium, 213-Bismut, 90-Ytrium and 212-Plumbum.

### 5.3 Chelators

Lists of typically applicable chelators and their short names are given below; Corresponding salts thereof are also applicable.:

Acetyl acetone (ACAC), ethylene diamine (EN), 2-(2-aminoethylamino)ethanol (AEEA), diethylene triamine (DIEN), iminodiacetate (IDA), triethylene tetramine (TRIEN), triaminotriethylamine, nitrilotriacetate (NTA) and its saltslike Na₃NTA or FeNTA, ethylenediaminotriacetate (TED), ethylenediamine tetraacetate (EDTA) and its salts like Na₂EDTA and CaNa₂EDTA, diethylene triaminpentaacetate (DTPA), 1,4,7,10-ztetraazacyclododecane-1,4,7,10-tetraacetate (DOTA), Oxalate (OX), tartrate (TART), citrate (CIT), dimethylglyoxime (DMG), 8-hydroxyquinoline, 2,2′-bipyridine (BPY), 1,10-phenanthroline (PHEN), dimercapto succinic acid (DMSA), 1,2-bis(diphenylphosphino)ethane (DPPE), sodium salicylate, methoxy salicylates, British anti-Lewisite or 2,3-dimercaprol (BAL), meso-2,3-dimercaptosuccinic acid (DMSA); Siderophores secreted by microorganisms, as for example desferrioxamine or deferoxamine B, also known as Deferral (Novartis), produced by Streptomyces spp.; deferoxamine (DFO), a trihydroxamic acid secreted by Streptomyces pilosus; phytochemicals like curcuminoids and derivatives of mugineic acid, like 3-hydroxy-mugineic acid and 2′-deoxy-mugineic acid; synthetically produced chelators, like Ibuprofen; derivatives of catechol, hydroxamate and hydroxypyridinone, like hydroxamate desferal and hydroxypyridinone deferiprone; deferiprone (L1 or 1,2-dimethyl-3-hydroxypyrid-4-one); D-penicillamine (DPA or D-PEN) which is β-β-dimethylcysteine or 3-mercapto-D-valine; tetraethylenetetraamine (TETA) or trientine and its two major metabolites N₁-acetyltriethylenetetramine (MAT) and N₁,N₁₀-diacetyltriethylenetetramine (DAT); hydroxyquinolines; clioquinol, which is a halogenated derivative of 8-hydroxyquinoline; and 5,7-dichloro-2-[(dimethylamino)methyl]quinolin-8-ol (PBT2)

### 6 UNAA

UNAAs useful in methods and kits of the present invention have been described in the prior art (for review see e.g. Liu et al., Annu Rev Biochem 83:379-408, 2010, Lemke, ChemBioChem 15:1691-1694, 2014).

The UNAAs may comprise a group (herein referred to as “labeling group”) that facilitates reaction with a suitable group (herein referred to as “docking group”) of another molecule (herein termed “conjugation partner molecule”) so as to covalently attach the conjugation partner molecule to the UNAA. When a UNAA comprising a labeling group is translationally incorporated into a POI, the labeling group becomes part of the POI. Accordingly, a POI prepared according to the method of the present invention can be reacted with one or more than one conjugation partner molecule such that the conjugation partner molecules bind covalently to the (labeling groups of the) unnatural amino acid residue(s) of the POI. Such conjugation reactions may be used for in situ coupling of POIs within a cell or tissue expressing the POI, or for site-specific conjugation of isolated or partially isolated POIs.

Particular useful choices for combinations of labeling groups and docking groups (of conjugation partner molecules) are those, which can react by metal-free click reactions. Such click reactions include strain-promoted inverse-electron-demand Diels-Alder cycloadditions (SPIEDAC; see, e.g., Devaraj et al., Angew Chem Int Ed Engl 2009, 48:7013)) as well as cycloadditions between strained cycloalkynyl groups, or strained cycloalkynyl analog groups having one or more of the ring atoms not bound by the triple bond substituted by amino groups), with azides, nitrile oxides, nitrones and diazocarbonyl reagents (see, e.g., Sanders et al., J Am Chem Soc 2010, 133:949; Agard et al., J Am Chem Soc 2004, 126:15046), for example strain promoted alkyne-azide cycloadditions (SPAAC). Such click reactions allow for ultrafast and biorthogonal covalent site-specific coupling of UNAA labeling groups of POIs with suitable groups of coupling partner molecule.

Pairs of docking and labeling groups which can react via the above-mentioned click reactions are known in the art. Examples of suitable UNAAs comprising docking groups include, but are not limited to, the UNAAs described, e.g., in WO 2012/104422 and WO 2015/107064.

Examples of particular suitable pairs of docking groups (comprised by the conjugation partner molecule) and labeling groups (comprised by the UNAA residue(s) of the POI) include but are not limited to:
(a) a docking group comprising (or essentially consisting of) a group selected from an azido group, a nitrile oxide functional group (i.e. a radical of formula, a nitrone functional group or a diazocarbonyl group, combined with a labeling group comprising (or essentially consisting of) an optionally substituted strained alkynyl group (such groups can react covalently in a copper-free strain promoted alkyne-azide cycloaddition (SPAAC));
(b) a docking group comprising (or essentially consisting of) an optionally substituted strained alkynyl group, combined with a labeling group comprising (or essentially consisting of) a group selected from an azido group, a nitrile oxide functional group (i.e. a radical of formula, a nitrone functional group or a diazocarbonyl group (such groups can react covalently in a copper-free strain promoted alkyne-azide cycloaddition (SPAAC));
(c) a docking group comprising (or essentially consisting of) a group selected from optionally substituted strained alkynyl groups, optionally substituted strained alkenyl groups and norbornenyl groups, combined with a labeling group comprising (or essentially consisting of) an optionally substituted tetrazinyl group (such groups can react covalently in a copper-free strain promoted inverse-electron-demand Diels-Alder cycloaddition (SPIEDAC)).
(d) a docking group comprising (or essentially consisting of) an optionally substituted tetrazinyl group, combined with a labeling group comprising (or essentially consisting of) a group selected from optionally substituted strained alkynyl groups, optionally substituted strained alkenyl groups and norbornenyl groups (such groups can react covalently in a copper-free strain promoted inverse-electron-demand Diels-Alder cycloaddition (SPIEDAC)).

Optionally substituted strained alkynyl groups include, but are not limited to, optionally substituted trans-cyclooctenyl groups, such as those described in. Optionally substituted strained alkenyl groups include, but are not limited to, optionally substituted cyclooctynyl groups, such as those described in WO 2012/104422 and WO 2015/107064. Optionally substituted tetrazinyl groups include, but are not limited to, those described in WO 2012/104422 and WO 2015/107064.

An azido group is a radical of formula —N₃.

A nitrone functional group is a radical of formula —C(R^{x})═N⁺(R^{y})—O⁻, wherein R^{x} and R^{y} are organic residues, e.g., residues independently selected from C₁-C₆-alkyl as described herein.

A diazocarbonyl group is a radical of formula —C(O)—CH═N₂.

A nitrile oxide functional group is a radical of formula —C≡N⁺—O⁻ or, preferably, of formula —C═N⁺(R^{x})—O⁻, wherein R^{x} is an organic residue, e.g., a residue selected from C₁-C₆-alkyl as described herein.

“Cyclooctynyl is an unsaturated cycloaliphatic radical having 8 carbon atoms and one triple bond in the ring structure.

“Trans-cyclooctenyl” is an unsaturated cycloaliphatic radical having 8 carbon atoms and one double bond that is in trans configuration in the ring structure.

“Tetrazinyl” is a 6-membered monocyclic aromatic radical having 4 nitrogen ring atoms and 2 carbon ring atoms.

Unless indicated otherwise, the term “substituted” means that a radical is substituted with 1, 2 or 3, especially 1 or 2, substituent(s). In particular embodiments, these substituents can be selected independently from hydrogen, halogen, C₁-C₄-alkyl, (R^{a}O)₂P(O)O—C₁-C₄-alkyl, (R^{b}O)₂P(O)—C₁-C₄-alkyl, CF₃, CN, hydroxyl, C₁-C₄-alkoxy, —O—CF₃, C₂-C₅-alkenoxy, C₂-C₅-alkanoyloxy, C₁-C₄-alkylaminocarbonyloxy or C₁-C₄-alkylthio, C₁-C₄-alkylamino, di-(C₁-C₄-alkyl)amino, C₂-C₅-alkenylamino, N—C₂-C₅-alkenyl-N—C₁-C₄-alkyl-amino and di-(C₂-C₅-alkenyl)amino, wherein R^{a} and R^{b} R^{a}, R^{b} are independently hydrogen or C₂-C₅-alkanoyloxymethyl.

The term halogen denotes in each case a fluorine, bromine, chlorine or iodine radical, in particular a fluorine radical.

C₁-C₄-Alkyl is a straight-chain or branched alkyl group having from 1 to 4, in particular from 1 to 3 carbon atoms. Examples include methyl and C₂-C₄-alkyl such as ethyl, n-propyl, iso-propyl, n-butyl, 2-butyl, iso-butyl and tert-butyl.

C₂-C₅-Alkenyl is a singly unsaturated hydrocarbon radical having 2, 3, 4 or 5 carbon atoms. Examples include vinyl, allyl (2-propen-1-yl), 1-propen-1-yl, 2-propen-2-yl, methallyl (2-methylprop-2-en-1-yl), 1-methylprop-2-en-1-yl, 2-buten-1-yl, 3-buten-1-yl, 2-penten-1-yl, 3-penten-1-yl, 4-penten-1-yl, 1-methylbut-2-en-1-yl and 2-ethylprop-2-en-1-yl.

C₁-C₄-Alkoxy is a radical of formula R—O—, wherein R is a C₁-C₄-alkyl group as defined herein.

C₂-C₅-Alkenoxy is a radical of formula R—O—, wherein R is C₂-C₅-alkenyl as defined herein.

C₂-C₅-Alkanoyloxy is a radical of formula R—C(O)—O—, wherein R is C₁-C₄-alkyl as defined herein.

C₁-C₄-Alkylaminocarbonyloxy is a radical of formula R—NH—C(O)—O—, wherein R is C₁-C₄-alkyl as defined herein.

C₁-C₄-Alkylthio is a radical of formula R—S—, wherein R is C₁-C₄-alkyl as defined herein.

C₁-C₄-Alkylamino is a radical of formula R—NH—, wherein R is C₁-C₄-alkyl as defined herein.

Di-(C₁-C₄-alkyl)amino is a radical of formula R^{x}—N(R^{y})—, wherein R^{x} and R^{y} are independently C₁-C₄-alkyl as defined herein.

C₂-C₅-Alkenylamino is a radical of formula R—NH—, wherein R is C₂-C₅-alkenyl as defined herein.

N—C₂-C₅-alkenyl-N—C₁-C₄-alkyl-amino is a radical of formula R^{x}—N(R^{y})—, wherein R^{x} is C₂-C₅-alkenyl as defined herein and R^{y} is C₁-C₄-alkyl as defined herein.

Di-(C₂-C₅-alkenyl)amino is a radical of formula R^{x}—N(R^{y})—, wherein R^{x} and R^{y} are independently C₂-C₅-alkenyl as defined herein.

C₂-C₅-Alkanoyloxymethyl is a radical of formula R^{x}—C(O)—O—CH₂—, wherein R^{x} is C₁-C₄-alkyl as defined herein.

The UNAAs used in the context of the present invention can be used in the form of their salt. Salts of an UNAA as described herein mean acid or base addition salts, especially addition salts with physiologically tolerated acids or bases. Physiologically tolerated acid addition salts can be formed by treatment of the base form of an UNAA with appropriate organic or inorganic acids. UNAAs containing an acidic proton may be converted into their non-toxic metal or amine addition salt forms by treatment with appropriate organic and inorganic bases. The UNAAs and salts thereof described in the context of the present invention also comprise the hydrates and solvent addition forms thereof, e.g. hydrates, alcoholates and the like.

Physiologically tolerated acids or bases are in particular those which are tolerated by the translation system used for preparation of POI with UNAA residues, e.g. are substantially non-toxic to living eukaryotic cells.

UNAAs, and salts thereof, useful in the context of the present the invention can be prepared by analogy to methods, which are well known in the art and are described, e.g., in the various publications cited herein.

The nature of the coupling partner molecule depends on the intended use. For example, the POI may be coupled to a molecule suitable for imaging methods or may be functionalized by coupling to a bioactive molecule. For instance, in addition to the docking group, a coupling partner molecule may comprise a group that selected from, but are not limited to, dyes (e.g. fluorescent, luminescent, or phosphorescent dyes, such as dansyl, coumarin, fluorescein, acridine, rhodamine, silicon-rhodamine, BODIPY, or cyanine dyes), molecules able to emit fluorescence upon contact with a reagent, chromophores (e.g., phytochrome, phycobilin, bilirubin, etc.), radiolabels (e.g. radioactive forms of hydrogen, fluorine, carbon, phosphorous, sulphur, or iodine, such as tritium, ¹⁸F, ¹¹C, ¹⁴C, ³²P, ³³P, ³³S, ³⁵S, ¹¹In, ¹²⁵I, ¹²³I, ¹³¹I, ²¹²B, ⁹⁰Y or ¹⁸⁶Rh), MRI-sensitive spin labels, affinity tags (e.g. biotin, His-tag, Flag-tag, strep-tag, sugars, lipids, sterols, PEG-linkers, benzylguanines, benzylcytosines, or co-factors), polyethylene glycol groups (e.g., a branched PEG, a linear PEG, PEGs of different molecular weights, etc.), photocrosslinkers (such as p-azidoiodoacetanilide), NMR probes, X-ray probes, pH probes, IR probes, resins, solid supports and bioactive compounds (e.g. synthetic drugs). Suitable bioactive compounds include, but are not limited to, cytotoxic compounds (e.g., cancer chemotherapeutic compounds), antiviral compounds, biological response modifiers (e.g., hormones, chemokines, cytokines, interleukins, etc.), microtubule affecting agents, hormone modulators, and steroidal compounds. Specific examples of useful coupling partner molecules include, but are not limited to, a member of a receptor/ligand pair; a member of an antibody/antigen pair; a member of a lectin/carbohydrate pair; a member of an enzyme/substrate pair; biotin/avidin; biotin/streptavidin and digoxin/antidigoxin.

The ability of certain (labeling groups of) UNAA residues to be coupled covalently in situ to (the docking groups of) conjugation partner molecules, in particular by a click reaction as described herein, can be used for detecting a POI having such UNAA residue(s) within a eukaryotic cell or tissue expressing the POI, and for studying the distribution and fate of the POIs. Specifically, the method of the present invention for preparing a POI by expression in eukaryotic cells can be combined with super-resolution microscopy (SRM) to detect the POI within the cell or a tissue of such cells. Several SRM methods are known in the art and can be adapted so as to utilize click chemistry for detecting a POI expressed by a eukaryotic cell of the present invention. Specific examples of such SRM methods include DNA-PAINT (DNA point accumulation for imaging in nanoscale topography; described, e.g., by Jungmann et al., Nat Methods 11:313-318, 2014), dSTORM (direct stochastic optical reconstruction microscopy) and STED (stimulated emission depletion) microscopy.

The following examples are illustrative only and are not intended to limit the scope of the embodiments described herein.

The numerous possible variations that will become immediately evident to a person skilled in the art after heaving considered the disclosure provided herein also fall within the scope of the invention.

### Experimental Part

Unless stated otherwise, the cloning steps carried out in the context of the present invention, for example restriction cleavage, agarose gel electrophoresis, purification of DNA fragments, transfer of nucleic acids onto nitrocellulose and nylon membranes, linkage of DNA fragments, transformation of microorganisms, culturing of microorganisms, multiplication of phages and sequence analysis of recombinant DNA are, if not otherwise sated, carried out by applying well-known techniques, as for example described in Sambrook et al. (1989) op. cit.

### A. Material and Methods

### Chemicals

TCO-E, TCO*A and SCO were purchased from SiChem (SIRIUS FINE CHEMICALS, SICHEM GMBH, Germany).

N^{ε}-tert-butyloxycarbonyl-L-lysine (Boc) was purchased from Iris Biotech GmbH, Germany).

### Cell Culture

HEK293T cells (ATCC CRL-3216) were cultivated in Dulbecco's modified Eagle's medium (DMEM, Gibco 41965-039) supplemented with 10% FBS (Sigma-Aldrich F7524), 1% penicillin-streptomycin (Sigma-Aldrich P0781), 1% L-Glutamine (Sigma-Aldrich G7513) and 1% sodium pyruvate (Life Technologies 11360). Cells were incubated at 37° C. and 5% CO₂, split every 2-3 days up to passage 20. HEK293T cells were seeded in a 24-well plate (Nunclon Delta Surface ThermoFisher SCIENTIFIC) at a cell density of 220.000 cells/ml, 500 μl/well. Cells were seeded 16 hours prior to transfection. For transfection, Dulbecco's modified Eagle's medium without Phenol-Red (DMEM, Gibco 11880-028) and as transfection reagent polyethylenimine (PEI, Sigma 408727) at a concentration of 1 mg/ml was used.

E. coli ElectroMAX™ DH10B F-mcrA Δ(mrr-hsdRMS-mcrBC) Φ80lacZΔM15 ΔlacX74 recA1 endA1 araD139Δ(ara, leu) 7697 galU galK λ⁻rpsL nupG (ThermoFisher Scientific, catalog number: 18290015)

### Culture Media

2×YT medium was prepared in house.

SOC medium was prepared in house.

### Cloning of Constructs

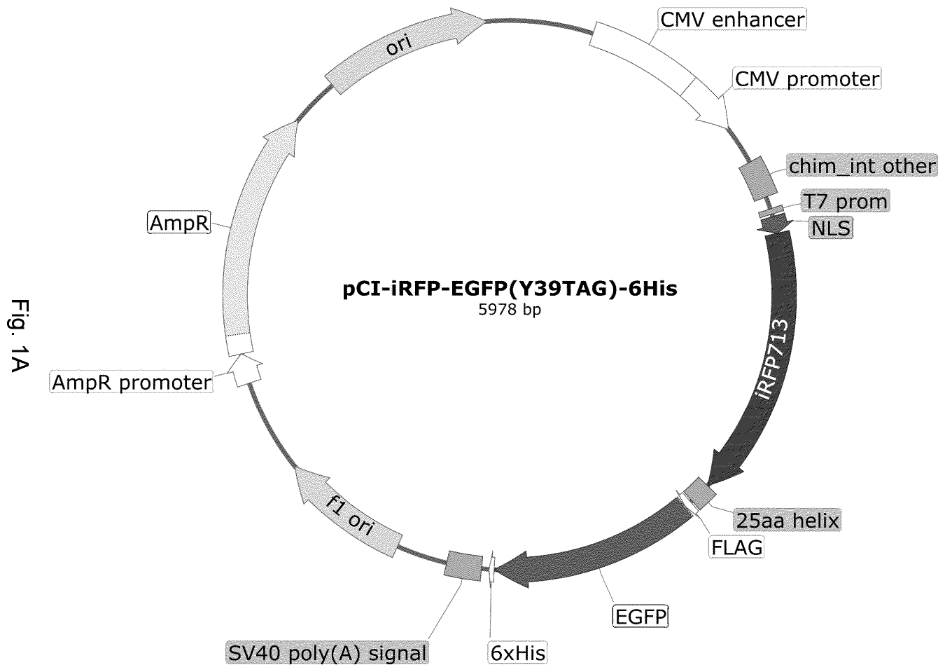

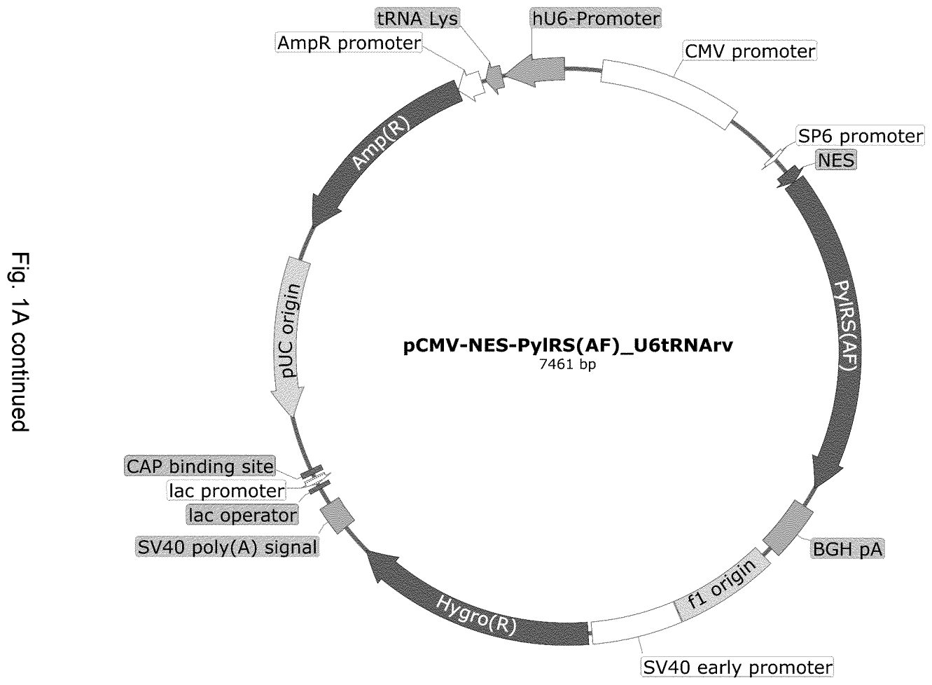

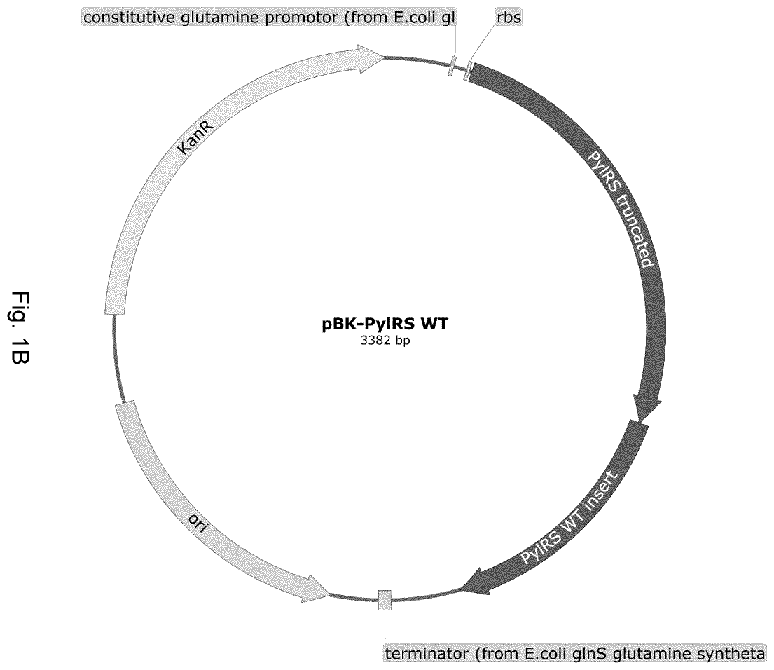

For the expression in mammalian cells, a reporter plasmid, pCI-iRFP-EGFP^{Y39TAG}-6His (SEQ ID NO: 97), was used, as published earlier (Nikić, I. et al. Debugging Eukaryotic Genetic Code Expansion for Site-Specific Click-PAINT Super-Resolution Microscopy. Angew. Chemie-Int. Ed. 55, 16172-16176 (2016) and shown in FIG. 1A, on top.

Plasmid pCMV-NES-PylRS^{AF}-U6tRNArv (SEQ ID NO: 98) is carrying the PylRS tRNA-synthetase from Methanosarcina mazei (Mm PylRS^{AF}), containing the Y306A and the Y384F mutation respectively and an N-terminal NES signal, as well as the tRNA expression cassette in an opposite direction, consisting of an U6 promoter signal and the Methanosarcina mazei tRNA^{Pyl} gene (FIG. 1A, bottom).

For the evolution of the synthetase, the PylRS genes were cloned into the pBK plasmid, a generous gift from Ryan Mehl (Cooley, R. B. et al. Structural Basis of Improved Second-Generation 3-Nitro-tyrosine tRNA Synthetases. Biochemistry 53, 1916-1924 (2014)). First, Methanosarcina mazei PylRS^{WT} was cloned into the pBK plasmid, using the restriction sites NdeI and PstI, after introducing a BglII site into the PylRS gene at bps 870-875, resulting into pBK-PylRS^{WT} plasmid (SEQ ID NO: 99) (FIG. 1B, top), containing a Kanamycin resistance cassette (Kan). The BglII site was further used together with PstI to place the PylRS gene library into this plasmid, by replacing this part of the PylRS^{WT} synthetase.

Plasmids used for the synthetase evolution, pREP-PylT (SEQ ID NO: 100) (FIG. 1B, bottom), pYOBB2-PylT (SEQ ID NO: 101) and pALS-sfGFP^{N150TAG}-MbPyl-tRNA (SEQ ID NO: 102) (FIG. 1C, top and bottom respectively) were generous gifts from the Ryan Mehl lab (Cooley, R. B. et al. Structural Basis of Improved Second-Generation 3-Nitro-tyrosine tRNA Synthetases. Biochemistry 53, 1916-1924 (2014); Porter, J. J. et al. Genetically Encoded Protein Tyrosine Nitration in Mammalian Cells. ACS Chem. Biol. 14, 1328-1336 (2019)).

The PylRS synthetase variants AF A1, AF B11, AF C11, AF G3 and AF H12, extracted from the synthetase selection procedure, were cloned out of the pBK plasmid and after digesting with BglII and PstI inserted into pCMV-NES-PylRS^{AF}-U6tRNArv, replacing the corresponding nucleotides of PylRS^{AF}.

### Site Directed Mutagenesis

To obtain the synthetase variant lacking the Y306A and Y384F mutations, these amino acids were mutated back to 306Y and 384Y by site-directed mutagenesis, resulting in the PylRS variant A1 by a two-step cloning using the primers PylRS A306Y fw (5′ aatctttataactatatgcgcaaactggaccgtgc 3′) (SEQ ID NO: 103) and PylRS A306Y rv (5′ atagttataaagatttggtgctagcatagggcgc 3′) (SEQ ID NO: 104), as well as the primer pair PylRS F384Y fw (5′ atggtgtatggcgacaccctggatgtcatg 3′) (SEQ ID NO: 105) and PylRS F384Y rv (5′ tgtcgccatacaccatacagctgtcgcccac 3′) (SEQ ID NO: 106).

### B. Examples

### Example 1: Evolution of PylRS Synthetase for Incorporation of TCO-E

A NNK synthetic library, which was based on the Methanosarcina mazei PylRS^{AF} variant (Y306A, Y384F) was ordered from GenScript Biotech Corp. with five sites (L305, L309, C348, I405 and W417) mutated to contain any of the 20 amino acids at these positions excluding two stop codons, which leads to 32 possible variants at each of the five sides, resulting in a 3.3×10⁷ library size. The library was cloned into the selection plasmid, pBK-PylRS^{WT}, replacing the binding pocket of PylRS with the library gene, resulting in pBK-PylRS^{lib}. For the selection pREP-PylT was transformed into E. coli DH10B cells and highly competent electro competent cells were prepared freshly, using LB medium with Tetracycline (Tet) to grow the cells up to OD₆₀₀ of 0.5. After harvesting the cells, they were washed with 10% Glycerol. To resuspend the cells after each harvesting step, the cells should be gently mixed by shaking the harvesting bottles, avoiding any pipetting step. The washing step was repeated two times and finally cells were taken up in as little volume as possible (for 1-liter expression in 1 ml 10% Glycerol) and aliquoted.

100 ng of pBK-PylRS^{lib} were transformed into 50 μl DH10B (pREP-PylT) cells by electroporation in a 1 mm cuvette and 800 μl of SOC medium were directly added. This step was repeated ten times and cells were combined into a 50 ml shaking flask to incubate for 1 hour at 37° C. shaking at 200 rpm. To estimate the library coverage after the transformation, LB-agar plates were prepared, containing Tet and Kan, and 10 μl of the cell suspension were used to make serial dilutions (1:10² to 1:10⁷). On each LB-agar plate 100 μl of dilution was plated and incubated overnight in a 37° C. incubator. The rest of the 10 ml transformation mixture was added into 500 ml 2×YT medium, containing Tet and Kan as antibiotics, and incubated over night at 37° C. on a shaker. To estimate the library coverage, 45 colonies on the 1:10⁶ plate were counted, which means 4.5×10⁹ total amount of cells. This yields in a 140-fold coverage of the library size.

Next day, the cells were diluted 1:100 in 500 ml fresh 2×YT medium (Tet, Kan) and grown up to OD₆₀₀ of 1, which normally took 2-4 hours. For the first positive selection, 10 LB-agar plates (150 mm petri dish) were prepared, containing 1 mM of TCO-E as well as 60 μg/ml Chloramphenicol (Cm), Tet and Kan. As control, one plate was prepared the same way, just without Cm. The plates were prepared under sterile conditions and cooled down until they were dry.

To start the selection, 100 μl of the culture were plated on each 150 mm LB-agar plate, spread by using glass beads and dried next to a flame. The plates were incubated overnight at 37° C., but not longer than 16 hours. The resulting colonies were scratched from plate with 5 ml 2×YT medium per plate and a cell scraper. The cell suspension of all ten plates was pooled into a 50 ml Erlenmeyer flask and shaken for 1 hour at 37° C. To isolate the library plasmid, the DNA was extracted first with a Miniprep Kit (Invitrogen) followed by gel extraction. Therefore, the DNA was loaded on a 1% agarose gel and the lowest band was cut. The DNA was isolated by a gel extraction kit (Invitrogen). The plasmid for the negative selection, pYOBB2-PylT, which contains a Barnase gen harboring two amber sites (Gln2 and Asp44), was transformed into DH10B cells and electro competent cells were prepared as mentioned above. 10 ng of gel extracted library plasmid were transformed into 50 μl freshly prepared DH10B (pYOBB2-PylT) cells and recovered after electroporation with 800 μl SOC medium for 1 hour at 37° C., shaking at 200 rpm in a 14 ml test tube. During the incubation time, the plates for the negative selection were prepared. Six 150 mm petri dishes were casted with LB-Agar, three of them containing 0.2% Arabinose to induce the Barnase expression. All of them containing 50 μg/ml Kan (pBK-PylRS^{lib} plasmid) and 33 μg/ml Cm (pYOBB2-PylT plasmid).

100 μl of the pure cells, as well as 100 μl of a 1:10 and 1:100 dilutions, were plated each on two plates (one with and one without Arabinose). After drying the plates next to a flame, they were incubated overnight in a 37° C. incubator. Colonies were scratched from plates and the DNA was extracted as mentioned above. The positive selection was repeated once more transforming 10 ng of gel extracted library plasmid in 50 μl of DH10B (pREP-PylT), recovering in 800 μl of SOC medium for 1 hour shaking at 37° C. This time only three 15 mm LB-agar plates containing 33 μg/ml Cm (Kan, Tet) with 1 mM TCO-E and one control plate without TCO-E were used for the selection. 100 μl of cell suspension were plated and incubated overnight at 37° C. The surviving colonies were scratched from plate and the library plasmid was extracted out of a 1% agarose gel.

To test the amber suppression efficiency of the remaining PylRS variants, an expression test based on superfolder GFP (sfGFP) was performed. Therefore, the library DNA was transformed together with the plasmid pALS-sfGFP^{N150TAG}-MbPyl-tRNA, encoding sfGFP with an amber site at position N150 and the tRNA^{Pyl} from Methanosarcina barkeri into DH10B cells. To include controls, the pBK-PylRS plasmid, encoding for PylRS^{WT} as well one encoding PylRS^{AF} were also co-transformed with the pALS-sfGFP^{N150TAG}-MbPyl-tRNA (SEQ ID NO: 102) plasmid. The transformations were recovered in SOC medium for 1 hour and different amounts (50, 100 and 200 μl) were plated on auto-inducing minimal media plates (Kan, Tet) with 1 mM TCO-E and without ncAA. Colonies were allowed to grow for 24 hours at 37° C. and if needed, another 24 hours at RT.

Green colonies were picked and grown overnight in a 96-well plate, containing 480 μl of non-inducing minimal media in each well. The controls (PylRS^{WT} and PylRS^{AF}) were also picked and included in this 96-well plate. Next day, two 96-well plates were prepared, each with 480 μl of auto-inducing minimal medium per well, one plate with 1 mM TCO-E and one plate without. 20 μl of each overnight culture were pipetted into corresponding wells in the new 96-well plates and incubated for 24 hours at 37° C. shaking at 250 rpm. The sfGFP expression was analyzed with a fluorescent measurement (BIOTEK Synergy 2 Microplate Reader). Therefore, a 96-well plate, containing 180 μl water per well was prepared, in which 20 μl of the sfGFP expression culture was pipetted in the corresponding well. To correct for different OD₆₀₀ of each expression, also an optical density scan at 600 nm was performed (150 μl water+50 μl expression culture).

Out of the 96-well plate with the non-inducing minimal media, each well of interest can be analyzed further, e.g. the DNA of the PylRS variant can be extracted for sequencing and further subcloning.

In this way the variants the PylRS synthetase variants AF A1, AF B11, AF C11, AF G3 and AF H12 were identified.

### Example 2: Estimation of Incorporation Efficiency of Different Synthetases Variants

These new PylRS variants as obtained from example 1 were tested in HEK293T cells utilizing a fluorescent reporter by fluorescent flow cytometry (FFC). The reporter contains an infrared fluorescent protein (iRFP) fused to enhanced green fluorescent protein (EGFP) which harbors an amber stop codon at position Y39 and is called iRFP-EGFP^{Y39TAG}. To analyze the incorporation efficiency for each new PylRS variant, transient transfection with different ncAAs and different amounts of ncAAs were performed.

Transfected cells can express iRFP and show a signal in the vertical axis of the FFC plots. If the PylRS variant can charge the tRNA with the corresponding ncAA, in this case TCO*A or TCO-E, there can also be a green signal observed resulting from EGFP expression in the horizontal axis.

The iRFP and EGFP signals were measured after 24 hours post transfection. Therefore, HEK293T cells were seeded in 24-well plate 16 hours prior to transfection. For the two-plasmid transfection, 1 μg of total DNA was used per well. The DNA was mixed with 50 μl of DMEM medium without Phenol Red and 3 μl of PEI were added. After 10 seconds of vortexing and a short centrifugation step, the DNA mixture was incubated 15 minutes in the hood, before it was added dropwise to the well. A master mix was prepared for all wells whenever suitable. After four hours of incubation, the medium was aspirated and fresh medium with different concentrations of ncAAs was added. The concentration of ncAA was 250 μM. After 20 hours of incubation, the iRFP and EGFP signal was analyzed with a flow cytometer analyzer (LSRFortessa™, BD Biosciences). Stock solutions for all ncAAs were prepared as described previously (Nikic, I., Kang, J. H., Girona, G. E., Aramburu, I. V. & Lemke, E. A. Labeling proteins on live mammalian cells using click chemistry. Nat. Protoc. 10, 780-791 (2015)).

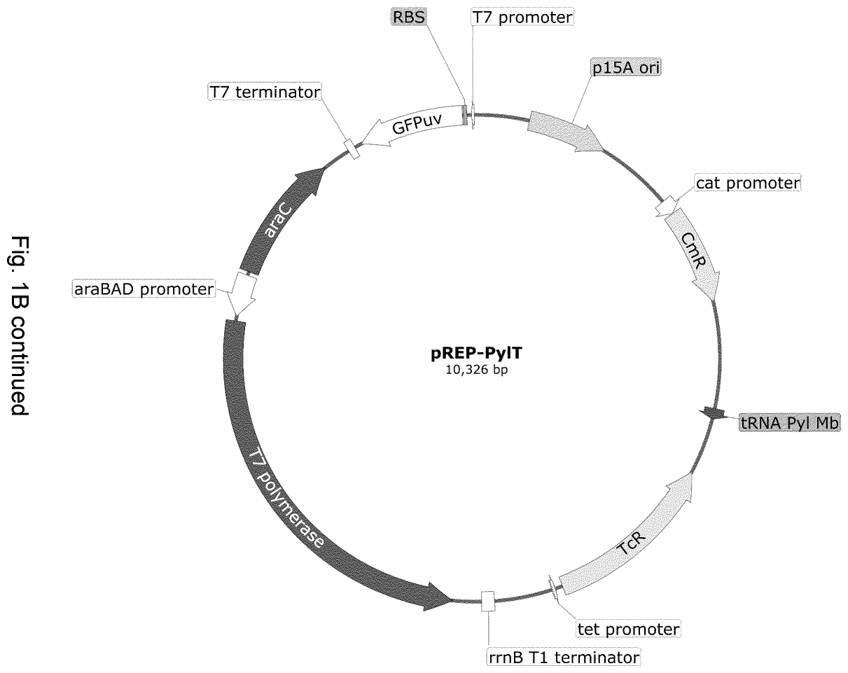

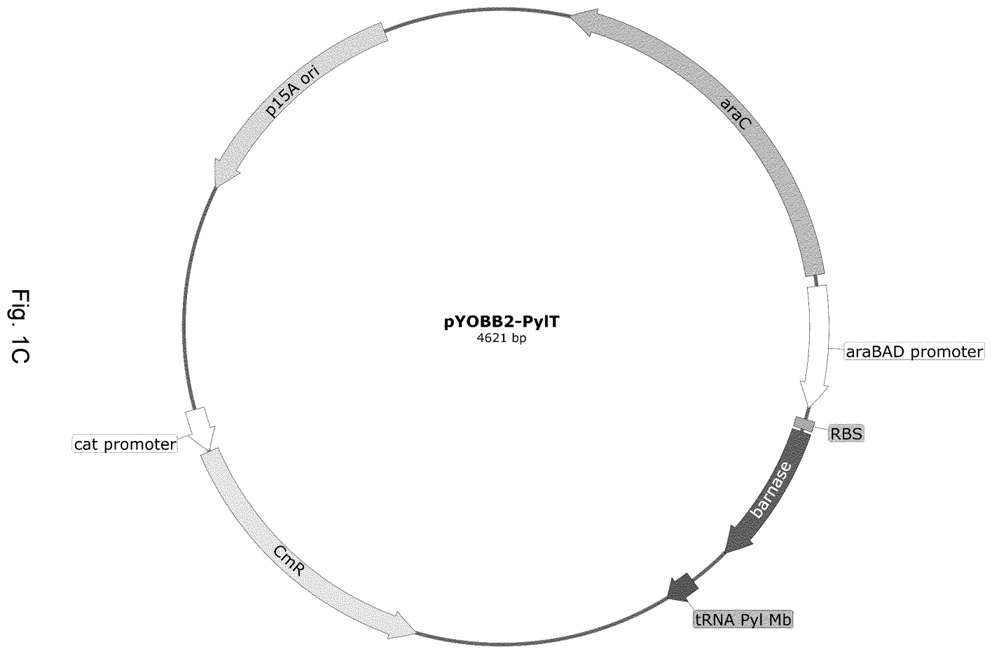

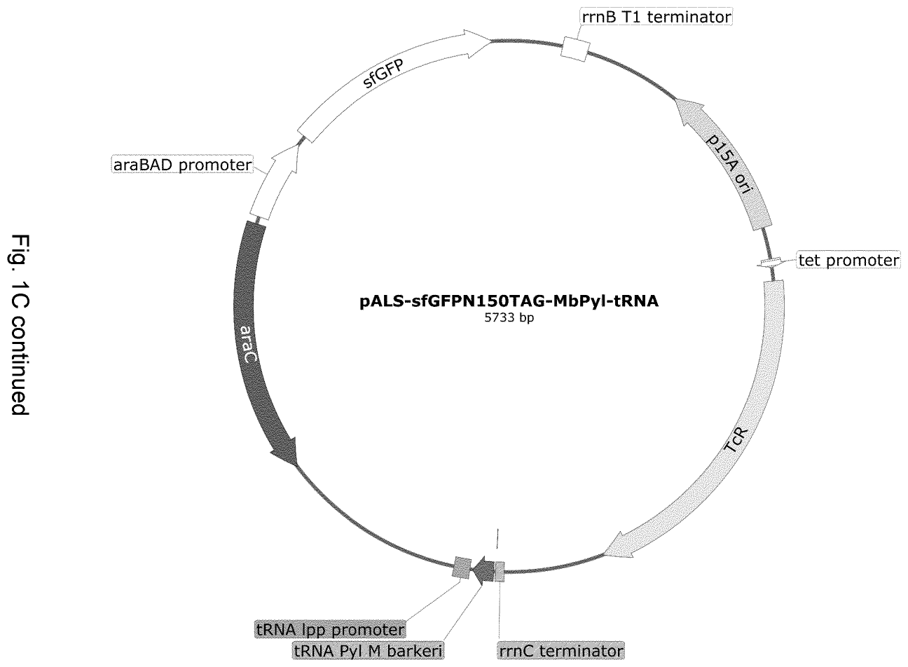

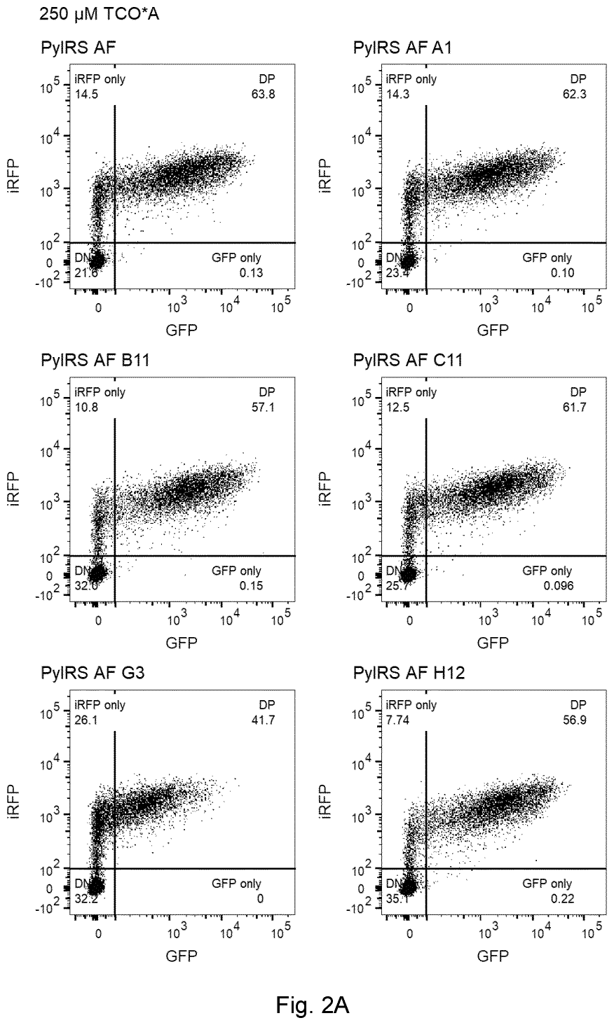

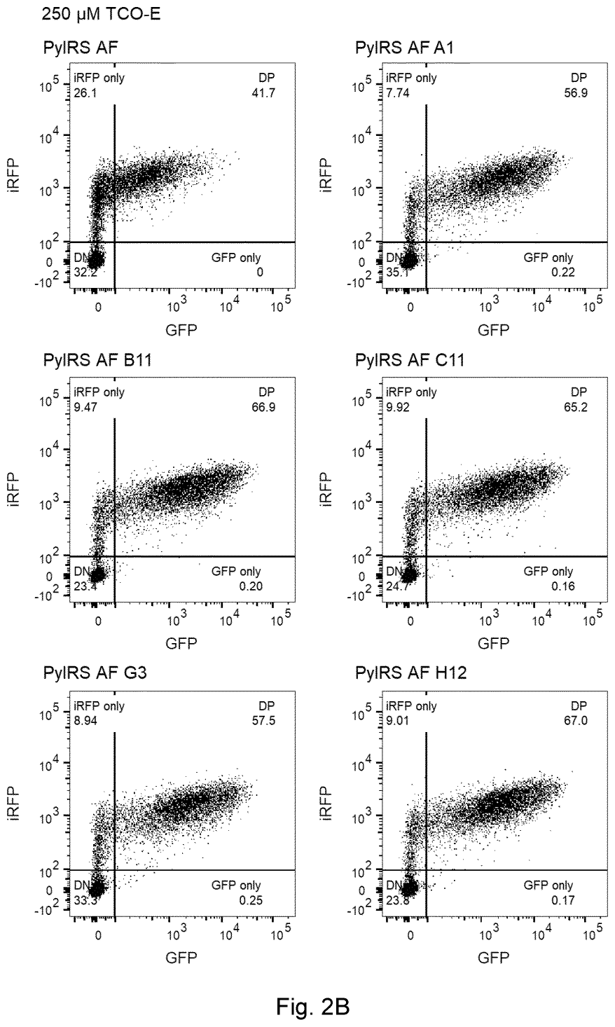

The results of these expression tests using different ncAAs are shown in FIG. 2. All of the new variants are able to incorporate TCO*A (FIG. 2A) as well as TCO-E (FIG. 2B) tested here. In addition, all the new variants show a higher green signal in comparison to the PylRS^{AF} when TCO-E is used. FIG. 2C shows the expression profile in the absence of ncAA.

### Example 3: Evaluating the Importance of Mutations Y306A and Y384F for Incorporation of Bulky ncAAs

To test, if the mutations Y306A and Y384F are really important for the incorporation of bulky ncAAs, these amino acids were changed back to their original amino acid residues by site directed mutagenesis in the case of the PylRS AF A1 variant. Therefore, the new variant does not contain the Y306A and Y384F mutations and is called PylRS A1.

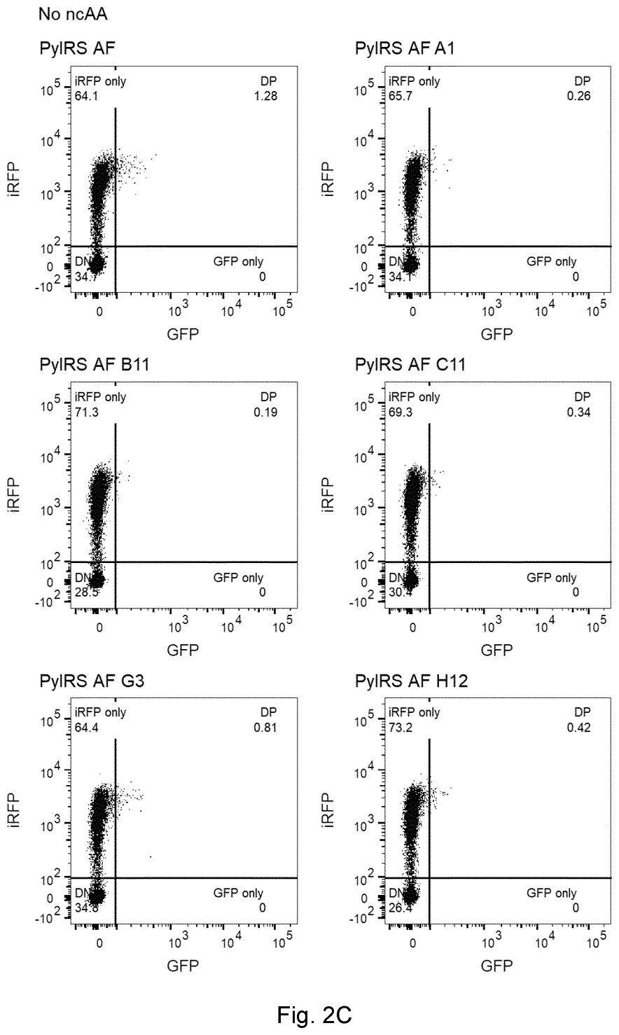

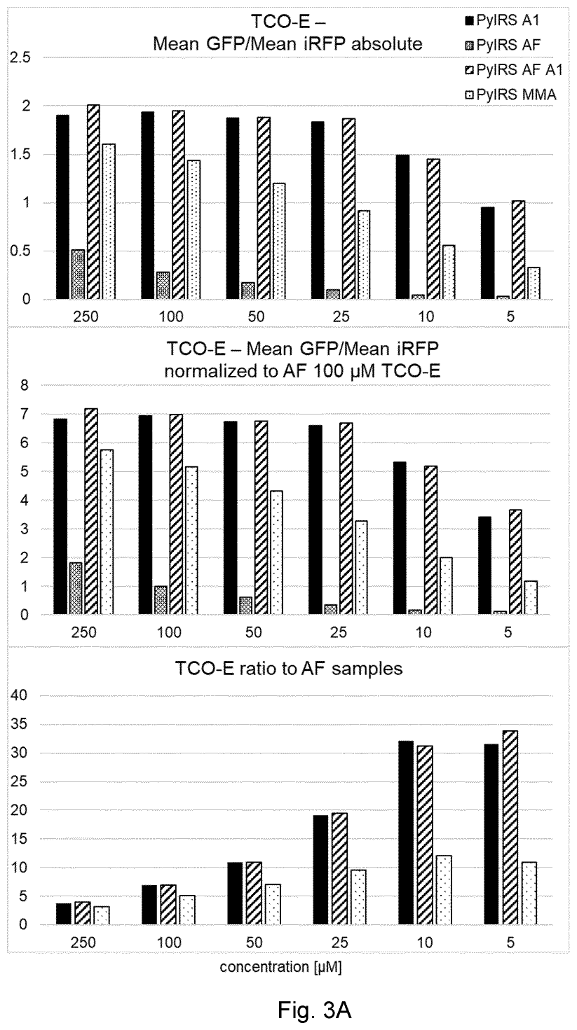

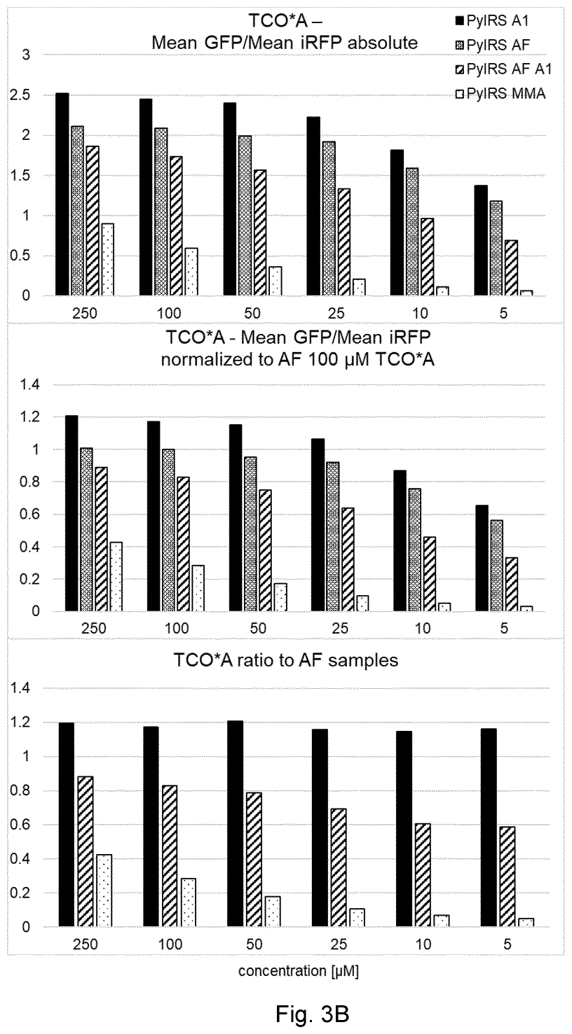

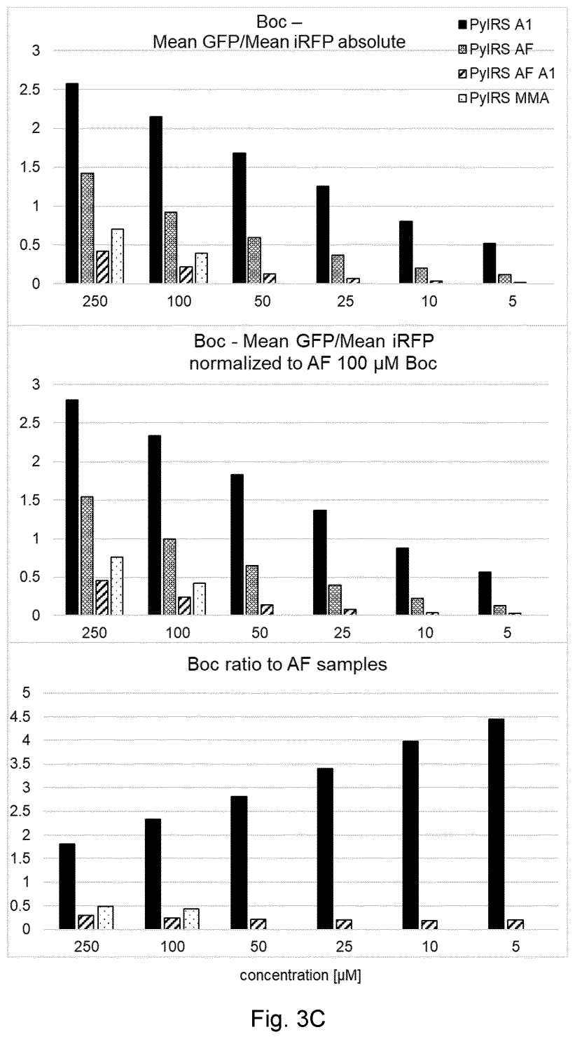

The FFC test was with TCO*A, TCO-E and in addition with Boc using the variants PylRS A1, PylRS AF, PylRS AF A1 and PylRS MMA. PylRS MMA has the mutations 306M 309M 348A. FIG. 3 illustrates the data obtained by FFC in bar plots for the different ncAAs used. The bar plot on top shows the ratio between the mean GFP signal obtained by FFC divided by the mean iRFP signal, which reflects the incorporation efficiency. The middle bar plot shows the same data but normalized to the ratio observed for the PylRS AF variant at 100 μM ncAA used. The bar plot on the bottom shows the ratio of mean GFP/mean iRFP normalized to the ratio mean GFP/mean iRFP obtained for the PylRS AF variant at the desired ncAA concentration. These bar plot illustrates on how much higher the incorporation efficiency is for each variant compared to PylRS AF.

All of the variants, also PylRS A1, are able to incorporate bulky ncAAs. This finding is unexpected and new for the community, because the Y306A and Y384F are always known to be the key mutations for the incorporation of bulky ncAAs. FIG. 3 A) illustrates the incorporation of TCO-E using the four different PylRS variants. The highest incorporation efficiency can be obtained by using PylRS A1 and PylRS AF A1, but not with PylRS AF. In FIG. 3 B) the incorporation efficiency is depicted by the FFC data using TCO*A as ncAA. In this case PylRS A1 shows the best incorporation. FIG. 3 C) shows the data for incorporation of Boc. Also here PylRS A1 can achieve the highest incorporation efficiency.

PylRS A1 can be up to 30 times better than PylRS AF in case of TCO-E depending on the concentration used and in case of TCO*A incorporation PylRS A1 shows a 1.2× higher efficiency independent of the ncAA concentration used. Also in case of Boc, PylRS A1 can show a higher incorporation efficiency up to 4.5× compared to PylRS AF.

In summary, the new variant PylRS A1 contains mutations which makes it the better synthetase for bulky ncAAs compared to the already known PylRS AF variant, which was unexpected accordingly to the literature.

In summary, the new variant PylRS MMA shows up to 10× higher incorporation efficiency of TCO-E in comparison to PylRS AF.

The content of any documents cross-referenced herein is incorporated by reference.

### Example 4: Evaluating the Incorporation Efficiency of Cyclooctyne-Lysine (SCO) by the New Variant PylRS A1

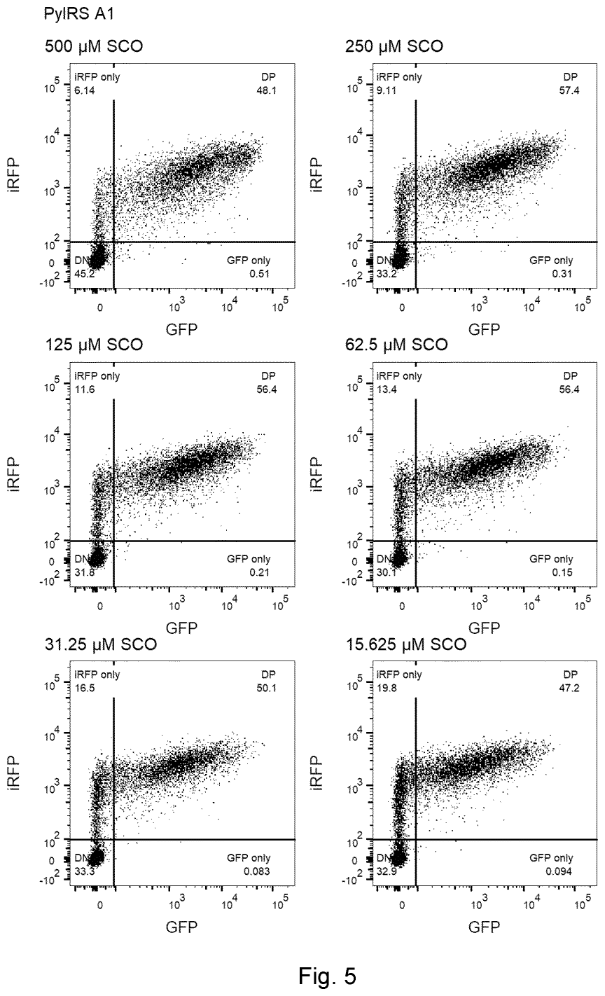

To test the incorporation efficiency of another ncAA by the new variant PylRS A1, an incorporation assay was performed utilizing HEK293T cells. The cells were co-transfected with a plasmid containing NES-PylRS A1 containing as well the corresponding tRNA^{Pyl} gene together with the reporter plasmid pCI-iRFP-EGFP^{Y39TAG}-6His (SEQ ID NO: 97). Different concentrations of SCO were added to the growth medium 4 hours after the transfection of the cells ranging from 15.625 μM to 500 μM SCO. After 20 hours of incubation, the iRFP and EGFP signal was analyzed with a flow cytometer analyzer (LSRFortessa™, BD Biosciences) as mentioned above. The corresponding FFC plots are shown in FIG. 5. The new variant A1 can incorporate SCO efficiently.

The content of the documents cross-referenced herein above is incorporated by reference.

### LISTING OF SEQUENCES

This listing is considered part of the general disclosure of the invention

| SEQ | | | | |
|---|---|---|---|---|
| ID NO | Description | Source | Feature | Type |
| 1 | Motif M1 | artificial | Conserved region | AA |
| 2 | Motif M2 | artificial | Conserved region | AA |
| 3 | Motif M3 | artificial | Conserved region | AA |
| 4 | Motif M4 | artificial | Conserved region | AA |
| 5 | Motif M5 | artificial | Conserved region | AA |
| 6 | Motif M6 | artificial | Conserved region | AA |
| 7 | Motif M1a | artificial | Conserved region | AA |
| 8 | Motif M1b | artificial | Conserved region | AA |
| 9 | Motif M1c | artificial | Conserved region | AA |
| 10 | Motif M1d | artificial | Conserved region | AA |
| 11 | Motif M1e | artificial | Conserved region | AA |
| 12 | Motif M1f | artificial | Conserved region | AA |
| 13 | Motif M2a | artificial | Conserved region | AA |
| 14 | Motif M2b | artificial | Conserved region | AA |
| 15 | Motif M2c | artificial | Conserved region | AA |
| 16 | Motif M2d | artificial | Conserved region | AA |
| 17 | Motif M2e | artificial | Conserved region | AA |
| 18 | Motif M2f | artificial | Conserved region | AA |
| 19 | Motif M3ab | artificial | Conserved region | AA |
| 20 | Motif M3c | artificial | Conserved region | AA |
| 21 | Motif M3d | artificial | Conserved region | AA |
| 22 | Motif M3e | artificial | Conserved region | AA |
| 23 | Motif M3f | artificial | Conserved region | AA |
| 24 | Motif M4a | artificial | Conserved region | AA |
| 25 | Motif M4b | artificial | Conserved region | AA |
| 26 | Motif M4c | artificial | Conserved region | AA |
| 27 | Motif M4d | artificial | Conserved region | AA |
| 28 | Motif M4e | artificial | Conserved region | AA |
| 29 | Motif M4f | artificial | Conserved region | AA |
| 30 | Motif M5ab | artificial | Conserved region | AA |
| 31 | Motif M5c | artificial | Conserved region | AA |
| 32 | Motif M5d | artificial | Conserved region | AA |
| 33 | Motif M5e | artificial | Conserved region | AA |
| 34 | Motif M5f | artificial | Conserved region | AA |
| 35 | Motif 6a-f | artificial | Conserved region | AA |
| 36 | Motif M1a* | artificial | Conserved region | AA |
| 37 | Motif M1b* | artificial | Conserved region | AA |
| 38 | Motif M1c* | artificial | Conserved region | AA |
| 39 | Motif M1d* | artificial | Conserved region | AA |
| 40 | Motif M1e* | artificial | Conserved region | AA |
| 41 | Motif M1f* | artificial | Conserved region | AA |
| 42 | Motif M2a* | artificial | Conserved region | AA |
| 43 | Motif M2b* | artificial | Conserved region | AA |
| 44 | Motif M2c* | artificial | Conserved region | AA |
| 45 | Motif M2d* | artificial | Conserved region | AA |
| 46 | Motif M2e* | artificial | Conserved region | AA |
| 47 | Motif M2f* | artificial | Conserved region | AA |
| 48 | Motif M4a* | artificial | Conserved region | AA |
| 49 | Motif M4b* | artificial | Conserved region | AA |
| 50 | Motif M4c* | artificial | Conserved region | AA |
| 51 | Motif M4d* | artificial | Conserved region | AA |
| 52 | Motif M4e* | artificial | Conserved region | AA |
| 53 | Motif M4f* | artificial | Conserved region | AA |
| 54 | NES | artificial | Nuclear export signal | AA |
| 55 | PylRS wt | Methanosarcina mazei, | Pyrrolysyl tRNA | NA |
| | | | Synthetase | |
| 56 | PylRS wt | Methanosarcina mazei, | Pyrrolysyl tRNA | AA |
| | | | Synthetase | |
| 57 | PylRS wt | Methanosarcina barkeri | Pyrrolysyl tRNA | NA |
| | | | Synthetase | |
| 58 | PylRS wt | Methanosarcina barkeri | Pyrrolysyl tRNA | AA |
| | | | Synthetase | |
| 59 | PylRS wt | Methanomethylophilus alvus, | Pyrrolysyl tRNA | NA |
| | | | Synthetase | |
| 60 | PylRS wt | Methanomethylophilus alvus, | Pyrrolysyl tRNA | AA |
| | | | Synthetase | |
| 61 | PylRS wt | Desulfitobacterium hafniense, | Pyrrolysyl tRNA | NA |
| | | | Synthetase | |
| 62 | PylRS wt | Desulfitobacterium hafniense, | Pyrrolysyl tRNA | AA |
| | | | Synthetase | |
| 63 | PylRS wt | Methanosarcinaceae archaeon | Pyrrolysyl tRNA | NA |
| 64 | PylRS wt | Methanosarcinaceae archaeon | Pyrrolysyl tRNA | AA |
| | | | Synthetase | |
| 65 | PylRS wt | Candidatus Methanoplasma | Pyrrolysyl tRNA | NA |
| | | termitum | Synthetase | |
| 66 | PylRS wt | Candidatus Methanoplasma | Pyrrolysyl tRNA | AA |
| | | termitum | Synthetase | |
| 67 | PylRS AF | Artificial (prior art) M. mazeii | Pyrrolysyl tRNA | NA |
| | | derived | Synthetase | |
| 68 | PylRS AF | Artificial (prior art) M. mazeii | Pyrrolysyl tRNA | AA |
| | | derived | Synthetase | |
| 69 | PylRS A1 | Artificial (Invention) M. mazeii | Pyrrolysyl tRNA | NA |
| | | derived | Synthetase | |
| 70 | PylRS A1 | Artificial (Invention) M. mazeii | Pyrrolysyl tRNA | AA |
| | | derived | Synthetase | |
| 71 | PylRS MMA | Artificial (Invention) M. mazeii | Pyrrolysyl tRNA | NA |
| | | derived | Synthetase | |
| 72 | PylRS MMA | Artificial (Invention) M. mazeii | Pyrrolysyl tRNA | AA |
| | | derived | Synthetase | |
| 73 | NES PylRS wt | Artificial M. mazeii | Pyrrolysyl tRNA | NA |
| | | derived + NES | Synthetase | |
| 74 | NES PylRS wt | Artificial M. mazeii | Pyrrolysyl tRNA | AA |
| | | derived + NES | Synthetase | |
| 75 | NES PylRS AF | Artificial M. mazeii | Pyrrolysyl tRNA | NA |
| | | derived + NES | Synthetase | |
| 76 | NES PylRS AF | Artificial M. mazeii | Pyrrolysyl tRNA | AA |
| | | derived + NES | Synthetase | |
| 77 | NES PylRS A1 | Artificial (Invention) M. mazeii | Pyrrolysyl tRNA | NA |
| | | derived + NES | Synthetase | |
| 78 | NES PylRS A1 | Artificial (Invention) M. mazeii | Pyrrolysyl tRNA | AA |
| | | derived + NES | Synthetase | |
| 79 | NES PylRS MMA | Artificial (Invention) M. mazeii | Pyrrolysyl tRNA | NA |
| | | derived + NES | Synthetase | |
| 80 | NES PylRS MMA | Artificial (Invention) M. mazeii | Pyrrolysyl tRNA | AA |
| | | derived + NES | Synthetase | |
| 81 | PylRS B11 | Artificial (Invention) M. mazeii | Pyrrolysyl tRNA | NA |
| | | derived | Synthetase | |
| 82 | PylRS B11 | Artificial (Invention) M. mazeii | Pyrrolysyl tRNA | AA |
| | | derived | Synthetase | |
| 83 | PylRS C11 | Artificial (Invention) M. mazeii | Pyrrolysyl tRNA | NA |
| | | derived | Synthetase | |
| 84 | PylRS C11 | Artificial (Invention) M. mazeii | Pyrrolysyl tRNA | AA |
| | | derived | Synthetase | |
| 85 | PylRS G3 | Artificial (Invention) M. mazeii | Pyrrolysyl tRNA | NA |
| | | derived | Synthetase | |
| 86 | PylRS G3 | Artificial (Invention) M. mazeii | Pyrrolysyl tRNA | AA |
| | | derived | Synthetase | |
| 87 | PylRS H12 | Artificial (Invention) M. mazeii | Pyrrolysyl tRNA | NA |
| | | derived | Synthetase | |
| 88 | PylRS H12 | Artificial (Invention) M. mazeii | Pyrrolysyl tRNA | AA |
| | | derived | Synthetase | |
| 89 | NES PylRS B11 | Artificial (Invention) M. mazeii | Pyrrolysyl tRNA | NA |
| | | derived + NES | Synthetase | |
| 90 | NES PylRS B11 | Artificial (Invention) M. mazeii | Pyrrolysyl tRNA | AA |
| | | derived + NES | Synthetase | |
| 91 | NES PylRS C11 | Artificial (Invention) M. mazeii | Pyrrolysyl tRNA | NA |
| | | derived + NES | Synthetase | |
| 92 | NES PylRS C11 | Artificial (Invention) M. mazeii | Pyrrolysyl tRNA | AA |
| | | derived + NES | Synthetase | |
| 93 | NES PylRS G3 | Artificial (Invention) M. mazeii | Pyrrolysyl tRNA | NA |
| | | derived + NES | Synthetase | |
| 94 | NES PylRS G3 | Artificial (Invention) M. mazeii | Pyrrolysyl tRNA | AA |
| | | derived + NES | Synthetase | |
| 95 | NES PylRS H12 | Artificial (Invention) M. mazeii | Pyrrolysyl tRNA | NA |
| | | derived + NES | Synthetase | |
| 96 | NES PylRS H12 | Artificial (Invention) M. mazeii | Pyrrolysyl tRNA | AA |
| | | derived + NES | Synthetase | |
| 97 | pCI-iRFP- | | Plasmid | NA |
| | GFP^{Y39TAG}-6His | | | |
| 98 | pCMV-NES- | | Plasmid | NA |
| | PylRS(AF)- | | | |
| | U6tRNArv | | | |
| 99 | pBK-PylRS WT | | Plasmid | NA |
| 100 | pREP-PylT | | Plasmid | NA |
| 101 | pYOBB2-PylT | | Plasmid | NA |
| 102 | pALS- | | Plasmid | NA |
| | sfGFPN150TAG- | | | |
| | MbPyl-tRNA | | | |
| 103 | PylRS A306Y fw | | PCR primer | NA |
| 104 | PylRS A306Y rv | | PCR primer | NA |
| 105 | PylRS F384Y fw | | PCR primer | NA |
| 106 | PylRS F384Y rv | | PCR primer | NA |
| 107 | PylRS AF A1 | Artificial M. mazeii | Pyrrolysyl tRNA | NA |
| | | derived | Synthetase | |
| 108 | PylRS AF A1 | Artificial M. mazeii | Pyrrolysyl tRNA | AA |
| | | derived | Synthetase | |
| 109 | NES PylRS AF A1 | Artificial M. mazeii | Pyrrolysyl tRNA | NA |
| | | derived + NES | Synthetase | |
| 110 | NES PylRS AF A1 | Artificial M. mazeii | Pyrrolysyl tRNA | AA |
| | | derived + NES | Synthetase | |
| 111 | NES pattern | artificial | Nuclear export signal | AA |
| 112 | NES pattern | artificial | Nuclear export signal | AA |
| 113 | tRNA^{pyl} | artificial | tRNA acylatable with | NA |
| | | | pyrrolysine | |

AA = Amino acid
NA = Nucleic acid

Also encompassed are variants of any of the above amino acid sequences lacking any N-terminal methionine residue and respective coding nucleic acid sequences.

1. A modified archaeal pyrrolysyl tRNA synthetase (PylRS), which comprises a combination of modified sequence motifs M1, M2, M3 and M4; optionally in combination with at least one further sequence motif selected from M5 and M6, and retains PylRS activity;
wherein
M1, M2, M3, M4, M5 and M6 are arranged in said order within the amino acid sequence of the modified PylRS with M1 being closest to the N-terminal end and M6 being closest to the C-terminal end of the sequence, and comprise the following sequences
M1:
(SEQ ID NO: 1)
LRPMX₁AX₂X₃L(Y/M)X₅X₆(M/V/C)R
M2:
(SEQ ID NO: 2)
HLX₇EFTMX₈NX₉(G/A)X₁₁X₁₂G
M3:
(SEQ ID NO: 3)
VYX₁₃X₁₄TX₁₅D
M4:
(SEQ ID NO: 4)
SX₁₆X₁₇X₁₈GP(R/I/N)X₂₀X₂₁D
M5:
(SEQ ID NO: 5)
X₂₂X₂₃(I/V)X₂₅X₂₆PW
M6:
(SEQ ID NO: 6)
G(A/L/I)GFGLERLL
wherein
amino acid residues X₁ to X₂₆ independently of each other are selected from naturally occurring amino acid residues.

2. The modified archaeal PylRS of claim 1, comprising a combination of sequence motifs selected from M1, M2, M3, M4 and M5; or M1, M2, M3, M4 and M6; or M1, M2, M3, M4, M5 and M6.

3. The modified archaeal PylRS of claim 1, which is derived from a parental PylRS originating from an archaeal bacterium of the genus Methanosarcina, Methanosarcinaceae, Methanomethylophilus, Desulfitobacterium, or Candidatus Methanoplasma.

4. The modified archaeal PylRS of claim 1, which is derived from a parental PylRS having an amino acid sequence selected from SEQ ID NO: 56, 58, 60, 62, 64 and 66 or a functional mutant or fragment thereof, which retains pyrrolysyl tRNA synthetase activity and has at least 60% sequence identity to the naturally occurring pyrrolysyl tRNA synthetase and which modified archaeal PylRS comprises a combination of modified sequence motifs M1, M2, M3 and M4; optionally in combination with at least one further sequence motif selected from M5 and M6, each as defined in claim 1.

5. The modified archaeal PylRS of claim 1, wherein
a) the sequence motif M1 is selected from the following sequences:
M1a:
(SEQ ID NO: 7)
LRPMLAPNLYNYMR
M1b:
(SEQ ID NO: 8)
LRPMLAPTLYNYMR
M1c:
(SEQ ID NO: 9)
LRPMLAPNLYSVMR
M1d:
(SEQ ID NO: 10)
LRPMLAPNLYTLMR
M1e:
(SEQ ID NO: 11)
LRPMLAPVLYNYMR
M1f:
(SEQ ID NO: 12)
LRPMHAMNLYYVMR
b) the sequence motif M2 is selected from the following sequences:
M2a:
(SEQ ID NO: 13)
HLEEFTMLNFGQMG
M2b:
(SEQ ID NO: 14)
HLEEFTMVNEGQMG
M2c:
(SEQ ID NO: 15)
HLEEFTMLNLGDMG
M2d:
(SEQ ID NO: 16)
HLNEFTMLNLGELG
M2e:
(SEQ ID NO: 17)
HLEEFTMVNFGQMG
M2f:
(SEQ ID NO: 18)
HLEEFTMLNLGELG
c) the sequence motif M3 is selected from the following sequences
M3a, b:
(SEQ ID NO: 19)
VYGDTLD
M3c:
(SEQ ID NO: 20)
VYKETID
M3d:
(SEQ ID NO: 21)
VYGDTVD
M3e:
(SEQ ID NO: 22)
VYGNTVD
M3f:
(SEQ ID NO: 23)
VYVETLD
d) the sequence motif M4 is selected from the following sequences
M4a:
(SEQ ID NO: 24)
SAVVGPRPLD
M4b:
(SEQ ID NO: 25)
SAVVGPRSLD
M4c:
(SEQ ID NO: 26)
SAAVGPRYLD
M4d:
(SEQ ID NO: 27)
SGAMGPRFLD
M4e:
(SEQ ID NO: 28)
SAVVGPRPMD
M4f:
(SEQ ID NO: 29)
SGAVGPRVLD
e) the sequence motif M5 is selected from the following sequences
M5a, b:
(SEQ ID NO: 30)
WGIDKPW
M5c:
(SEQ ID NO: 31)
HDIHEPW
M5d:
(SEQ ID NO: 32)
WEIFDPW
M5e:
(SEQ ID NO: 33)
WGINKPW
M5f:
(SEQ ID NO: 34)
HDIHEPW
f) the sequence motif M6 is selected from the following sequences
M6a, b, c, d, e, f:
(SEQ ID NO: 35)
GAGFGLERLL.

6. The modified archaeal PylRS of claim 1, wherein
a) the sequence motif M1 is selected from the following sequences:
M1a*:
(SEQ ID NO: 36)
LRPMLAPNLMNYMR
M1b*:
(SEQ ID NO: 37)
LRPMLAPTLMNYMR
M1c*:
(SEQ ID NO: 38)
LRPMLAPNLMSVMR
M1d*:
(SEQ ID NO: 39)
LRPMLAPNLMTLMR
M1e*:
(SEQ ID NO: 40)
LRPMLAPVLMNYMR
M1f*:
(SEQ ID NO: 41)
LRPMHAMNLMYVMR
b) the sequence motif M2 is selected from the following sequences:
M2a*:
(SEQ ID NO: 42)
HLEEFTMLNFAQMG
M2b*:
(SEQ ID NO: 43)
HLEEFTMVNFAQMG
M2c*:
(SEQ ID NO: 44)
HLEEFTMLNLADMG
M2d*:
(SEQ ID NO: 45)
HLNEFTMLNLAELG
M2ex:
(SEQ ID NO: 46)
HLEEFTMVNFAQMG
M2f*:
(SEQ ID NO: 47)
HLEEFTMLNLAELG
c) the sequence motif M3 is selected from the following sequences
M3a, b:
(SEQ ID NO: 19)
VYGDTLD
M3c:
(SEQ ID NO: 20)
VYKETID
M3d:
(SEQ ID NO: 21)
VYGDTVD
M3e:
(SEQ ID NO: 22)
VYGNTVD
M3f:
(SEQ ID NO: 23)
VYVETLD
d) the sequence motif M4 is selected from the following sequences
M4a*:
(SEQ ID NO: 48)
SAVVGPIPLD
M4b*:
(SEQ ID NO: 49)
SAVVGPISLD
M4c*:
(SEQ ID NO: 50)
SAAVGPIYLD
M4d*:
(SEQ ID NO: 51)
SGAMGPIFLD
M4e*:
(SEQ ID NO: 52)
SAVVGPIPMD
M4f*:
(SEQ ID NO: 53)
SGAVGPIVLD
e) the sequence motif M5 is selected from the following sequences
M5a, b:
(SEQ ID NO: 30)
WGIDKPW
M5c:
(SEQ ID NO: 31)
HDIHEPW
M5d:
(SEQ ID NO: 32)
WEIFDPW
M5e:
(SEQ ID NO: 33)
WGINKPW
M5f:
(SEQ ID NO: 34)
HDIHEPW
f) the sequence motif M6 is selected from the following sequences
M6a, b, c, d, e, f:
(SEQ ID NO: 35)
GAGFGLERLL.

7. The modified archaeal PylRS of claim 5 comprising a combination of sequence motifs M1a, M2a, M3a, M4a, M5a and M6a.

8. The modified archaeal PylRS of claim 1 which is
a) PylRS A1 comprising an amino acid sequence of SEQ ID NO: 70; or an amino acid sequence having a degree of sequence identity of at least 60% to SEQ ID NO: 70; or a functional fragment thereof retaining PylRS activity; or
b) PylRS MMA comprising an amino acid sequence of SEQ ID NO:72; or an amino acid sequence having a degree of sequence identity of at least 60% to SEQ ID NO: 72; or a functional fragment thereof retaining PylRS activity;
or
c) PylRS B11 comprising an amino acid sequence of SEQ ID NO:82; or an amino acid sequence having a degree of sequence identity of at least 60% to SEQ ID NO:82; or a functional fragment thereof retaining PylRS activity;
d) PylRS C11 comprising an amino acid sequence of SEQ ID NO:84; or an amino acid sequence having a degree of sequence identity of at least 60% to SEQ ID NO:84; or a functional fragment thereof retaining PylRS activity;
e) PylRS G3 comprising an amino acid sequence of SEQ ID NO:86; or an amino acid sequence having a degree of sequence identity of at least 60% to SEQ ID NO:86; or a functional fragment thereof retaining PylRS activity;
f) PylRS H12 comprising an amino acid sequence of SEQ ID NO:88; or an amino acid sequence having a degree of sequence identity of at least 60% to SEQ ID NO:88; or a functional fragment thereof retaining PylRS activity.

9. The modified archaeal PylRS of claim 1, which shows at least one of the following functional features:
a) Modified substrate profile for non-canonical amino acids (ncAAs); and
b) Improved utilization of at least one bulky ncAA, each relative to mutant PylRS AF (SEQ ID NO: 68)
c) Improved utilization of at least one bulky ncAA, each relative to mutant PylRS AF A1 (SEQ ID NO:108).

10. The modified archaeal PylRS of claim 8a), which shows at least one of the following functional features
a) Improved utilization of at least one bulky ncAA, selected from TCO-E, TCO*A and Boc, each relative to mutant PylRS AF (SEQ ID NO:68)
b) Improved utilization of at least one bulky ncAA, selected from, TCO*A and Boc, each relative to mutant PylRS AF A1 (SEQ ID NO:108).

11. The modified archaeal PylRS of claim 8b), which shows at least the following functional feature: improved utilization of the bulky ncAA TCO-E relative to mutant PylRS AF (SEQ ID NO:68).

12. The modified archaeal PylRS of claim 1, that comprises a nuclear export signal (NES).

13. A modified polynucleotide encoding the modified archaeal pyrrolysyl tRNA synthetase of claim 1.

14. The polynucleotide of claim 13, further encoding a tRNA^{Pyl}, wherein the tRNA^{Pyl} is a tRNA that can be acylated by the pyrrolysyl tRNA synthase encoded by polynucleotide of claim 13.

15. A combination of polynucleotides comprising at least one polynucleotide of claim 13 and at least one polynucleotide encoding a tRNA^{Pyl}, wherein the tRNA^{Pyl} is a tRNA that can be acylated by the pyrrolysyl tRNA synthase encoded by polynucleotide of claim 13.

16. The polynucleotide of claim 14, wherein the anticodon of the tRNA^{Pyl} is the reverse complement of a codon that is selected from stop codons, four base codons and rare codons.

17. A eukaryotic cell, comprising:
(a) a polynucleotide sequence encoding the pyrrolysyl tRNA synthetase of claim 1, and
(b) a tRNA that can be acylated by the pyrrolysyl tRNA synthase encoded by the sequence of (a), or a polynucleotide sequence encoding such tRNA.

18. A method for preparing a protein of interest (POI) comprising one or more than one unnatural amino acid residue, wherein the method comprises:
(a) Providing a eukaryotic cell comprising:
(i) a pyrrolysyl tRNA synthetase of claim 1,
(ii) a tRNA (tRNA^{Pyl}),
(iii) an unnatural amino acid or a salt thereof, and
(iv) a polynucleotide encoding the POI, wherein any position of the POI occupied by an unnatural amino acid residue is encoded by a codon that is the reverse complement of the anticodon comprised by the tRNA^{Pyl}; and
wherein the pyrrolysyl tRNA synthetase (i) is capable of acylating the tRNA^{Pyl} (ii) with the unnatural amino acid or salt (iii); and
(b) allowing for translation of the polynucleotide (iv) by the eukaryotic cell, thereby producing the POI.

19. A method for preparing a polypeptide conjugate comprising:
(a) preparing a POI comprising one or more than one unnatural amino acid residue using the method of claim 18; and
(b) reacting the POI with one or more than one conjugation partner molecule such that the conjugation partner molecules bind covalently to the unnatural amino acid residue(s) of the POI.

20. A kit comprising at least one unnatural amino acid, or a salt thereof and:
a polynucleotide of claim 13;
wherein the archaeal pyrrolysyl tRNA synthetase is capable of acylating tRNA^{Pyl} with the unnatural amino acid or salt thereof.

21. A kit comprising at least one unnatural amino acid, or a salt thereof and:
the combination of polynucleotides of claim 15,
wherein the archaeal pyrrolysyl tRNA synthetase is capable of acylating tRNA^{Pyl} with the unnatural amino acid or salt thereof.

22. A kit comprising at least one unnatural amino acid, or a salt thereof and:
the eukaryotic cell of claim 17,
wherein the archaeal pyrrolysyl tRNA synthetase is capable of acylating tRNA^{Pyl} with the unnatural amino acid or salt thereof.

23. The modified archaeal PylRS of claim 6 comprising a combination of sequence motifs M1a*, M2a*, M3a, M4a*, M5a and M6a.

24. The combination of polynucleotides of claim 15, wherein the anticodon of the tRNAPyl is the reverse complement of a codon that is selected from stop codons, four base codons and rare codons
